(19) United States
(12) Patent Application Publication
(10) Pub. No.: US 2022/0323663 A1
(43) Pub. Date: Oct. 13, 2022

(54) Cannula Comprising an Expandable Arrangement, Corresponding Cannula System and Method for Inserting at Least One Cannula into a Subject

(71) Applicant: ReCO2very Therapies GmbH, Bad Klosterlausnitz (DE)

(72) Inventor: Torsten HEILMANN, Bad Klosterlausnitz (DE)

(21) Appl. No.: 17/635,601

(22) PCT Filed: Aug. 19, 2020
(86) PCT No.: PCT/EP2020/073257

§ 371 Date: Feb. 15, 2022

(30) Foreign Application Priority Data
Aug. 19, 2019 (EP) PCT/EP2019/072165
Dec. 10, 2019 (EP) PCT/EP2019/084482

(51) Int. Cl. A61M 1/36; A61M 25/04; A61M 60/268; A61M 60/37

(52) U.S. Cl. CPC A61M 1/3661; A61M 25/04; A61M 60/268; A61M 60/37

(57) ABSTRACT

Described is a cannula (110, O1 to O3, I1 to I3) comprising: —a lumen portion (LP) that extends axially between a proximal part of the cannula (110, O1 to O3, I1 to I3) and at least one distal part of the cannula (110, O1 to O3, I1 to I3), and —an expandable arrangement (114) at the at least one distal part of the lumen portion, wherein the expandable arrangement (114) is adapted to have an expanded state and a non-expanded state, wherein in the expanded state a volume defined by the expandable arrangement (114) is greater than the volume defined by the expandable arrangement (114) in the non-expanded state.

Cannula comprising an expandable arrangement, corresponding cannula system and method for inserting at least one cannula into a subject

It is conceivable to have an inflatable expandable arrangement. However, there may be application scenarios in which an inflatable arrangement is not appropriate for use within the vascular system of a subject, especially within the body of a human or of an animal.

Furthermore, it may be conceivable to use an expandable arrangement that comprises wires. However, the question is how to arrange the wires in order to allow a variety of new applications for medical use.

It is an object of the invention to provide a cannula comprising an expandable arrangement, preferably a cannula that is easy to insert endovascular and/or that opens new application possibilities for cannulizing a subject. Preferably, the risk of injuries of during insertion and or removal of the catheters/cannulas shall be minimized, especially the risk to injure vessels through which the cannula is inserted. Furthermore, the invention relates to a corresponding cannula system, preferably to a multi-lumen or dual-lumen cannula system and to corresponding methods.

This object is solved by a cannula system according to claim 1. Embodiments are disclosed in the sub claims. Furthermore, the object is solved by a cannula system and a method. Embodiments are disclosed in the sub claims.

The cannula may comprise:
a lumen portion that extends axially between a proximal part of the cannula and a distal part of the cannula, and
an expandable arrangement at the distal part of the lumen portion, for instance comprising at least one balloon and or several wires,
   wherein the expandable arrangement may be adapted to have an expanded state and a non-expanded state, and
   wherein in the expanded state a volume defined by the expandable arrangement may be greater than the volume defined by the expandable arrangement in the non-expanded state.

The cannula may be made as thick as possible to allow high fluid flows within the lumen. However, the cannula may be made not too thick in order to reduce friction between the outer surface of the cannula and the inner surface of the vessels during insertion of the cannula. Therefore, the expandable arrangement may allow to fix the distal part of the cannula within in vessel or chamber of the body that has an inner diameter that is greater than the diameter or width of a distal part of the cannula but may be smaller than the diameter or with of the expandable arrangement.

Moreover, the expandable arrangement may fulfill at least one further function in addition or alternatively to fixation, for instance at least one, at least two or all of the following:
holding a membrane, and/or
preventing that a surrounding wall of a vessel is sucked into the cannula for a drainage cannula, and/or
preventing the “sand blasting” effect for a delivery cannula, e.g. destruction of tissue of a vessel by the pressure of a fluid, for instance blood, that comes out of the cannula, and/or
allowing high flow rates for inflow and/or outflow of a fluid, and/or
allowing the introduction of medical devices through the expandable arrangement, etc.

These functions are explained in more detail below together with the corresponding sub claims.

The lumen portion may be flexible and/or have an appropriate elasticity.

The cannula may comprise an optional tip portion of cannula, for instance made of different material and/or being more rigid than the lumen portion. The tip portion may have several holes or only one hole through which fluid can flow through into or out of the lumen portion.

In the following the longitudinal axis of lumen portion or the extension thereof beyond the lumen portion may be used as a reference axis. The terms “radial”, “axial” and/or “angularly” may be used with regard to this reference axis, similarly to cylinder coordinates are used in a cylindrical coordinate system.

The volume that is defined by the expandable arrangement may be an inner volume of the expandable arrangement, e.g. volume that is embraced or encompassed by the expandable arrangement.

The lumen portion may be the portion of the cannula that is insertable into a body of a subject, preferably endovascular, e.g. without surgery and mainly by using needles, dilators, guide wires, cannulas and/or introducer members.

In this application document the definition for “distal” is far from a person that inserts the cannula/catheter. “Proximal” near to the person that inserts the cannula.

The basic principle of an endovascular catheter/cannula therapy may be a treatment of vessels and/or by using vessels for the advancement of a catheter, for instance plastic tubes or plastic tubes that are armed with metal. An incision may be made into the skin of a patient. The incision may have a length that is less than 5 cm (centimeter), less than 3 cm or less than 1 cm. Local anesthesia may be used thereby. A cannula may be used to insert a guide wire and/or dilators to expand the incision and/or an opening within the vessel. The catheter may be inserted using the guide wire and/or an introducing member.

No thoracotomy may be necessary if cannulas or catheters are used. A cannula may be a tube that can be inserted into the body, often for the delivery or removal of fluid or for the gathering of data. A catheter may be a thin tube made from medical grade materials serving a broad range of functions. Catheters may be medical devices that can be inserted in the body to treat diseases or perform a surgical procedure. Both terms are used interchangeable in the following if not stated otherwise.

By modifying the material or adjusting the way cannulas/catheters are manufactured, it is possible to tailor them for cardiovascular, urological, gastrointestinal, neurovascular, and ophthalmic applications. A catheter/cannula left inside the body, either temporarily or permanently, may be referred to as an “indwelling catheter/cannula” (for example, a peripherally inserted central catheter/cannula).

Catheters and cannulas may be inserted into a body cavity, duct, or vessel. Functionally, they allow drainage, administration of fluids or gases, access by surgical instruments, and/or also perform a wide variety of other tasks depending on the type of catheter or cannula. The process of inserting a catheter is “catheterization”. The process of inserting a cannula is “cannulization”. In most uses, a catheter/cannula is a thin, flexible tube (“soft”) though catheters/cannulas are available in varying levels of stiffness depending on the application.

The lumen portion may be less expandable than the expandable arrangement, e.g. the lumen portion may be flexible but the diameter may not be expandable into two directions that include an angle of 90 degrees at the same time. The volume in the expanded state may be at least by factor 1.5 or 2, 3 or 5 greater than the volume in the non-expanded state. Furthermore, the maximal diameter may be greater in the expanded state by the same factor compared to the maximal diameter of the expandable arrangement in the non-expanded state. The volume in the expanded state may be less than factor 100 or less than factor 50 compared to the volume that is encompassed by the expandable arrangement in the non-expanded state.

The lumen portion may be adapted to guide an introducer member, for instance a long rod. The expandable arrangement may comprise a contact area that is adapted to have mechanical contact with the introducer member. The expandable arrangement may be configured such that it changes from the non-expanded state to the expanded state if the introducer member is moved away from the contact area. The distal part of the lumen portion may comprise a first opening that is adapted to allow the passage of the introducer member. The contact part of the expandable arrangement may extend at least in the non-expanded state to an axial position on the extended longitudinal axis of the lumen portion and to at least one radial position at the axial position where it prevents the passage of the introducer. The contact area may be opposite to an opening of the lumen portion. An introducer that is arranged within the cannula does not increase the outer diameter of the cannula. Thus, insertion is of the cannula into vessels is eased. The contact part of the expandable arrangement may extend at least in the non-expanded state to an axial position within the lumen portion. Alternatively or additionally, an external sheath may be used to hold the expandable arrangement in the non-expanded state.

The expandable arrangement may comprise a plurality of wires which may be connected to the lumen portion in a connection region of the respective wire. The usage of wires is an appropriate way to realize the two different state of the expandable arrangement. There are many ways to connect the wires in the connection region with the lumen portion. The wires may not extend into the lumen portion but may be separate parts with regard to the lumen portion.

At least two wires, at least three wires or all wires of the plurality of wires may be connected with each other and/or with a common connection element in an end region of the respective wire remote from the connection region as seen along the extension of the respective wire. The end region may also be used as the contact area for an introducer member. Thus, the end region may have multi functions.

In the expanded state, at least two wires, at least three wires or all wires of the plurality of wires may extend between the connection region and the end region of the respective wire without mechanical contact to other wires of the expandable arrangement and/or without crossing other wires of the expandable arrangement and/or without being crossed by other wires of the expandable arrangement. This arrangement allows the usage of a small number of wires compared for instance with a woven arrangement. Furthermore, the distance between angularly adjacent wires may be used for several purposes. A fluid flow through the expandable arrangement may have a high flow rate.

In the expanded state, at least two wires, at least three wires or all wires of the plurality of wires may have the same or similar shape in the expanded state and/or before assembly of the expandable arrangement. Thus, part logistics and/or storage is simple.

In the non-expanded state, at least two wires, any two arbitrarily selected wires or all wires of the plurality of wires may have the same or similar shape.

A sequence of angularly consecutive wires, preferably a sequence that comprises all wires of the plurality of wires, may comprise the same axial offset between two angularly adjacent wires and/or the same angularly offset between two angularly adjacent wires. Thus, assembly of the cage arrangement may be simple. If the wires are arranged appropriately a fixation step may follow that fixes the wires to each other, to an additional fixation part or directly to the lumen portion.

Similar or same shape may refer to the shape and/or area of cross sections and/or to the same special course within the three-dimensional space, i.e. same bending etc.

At least one wire, at least two wires or all wires of the plurality of wires has or have a portion of the respective wire that extends in the connection region of the wire angularly, preferably only angularly, along or around the outer circumference of the lumen portion. In a first alternative the connection region may extend less than 360 degrees around the lumen portion. Alternatively, the connection region may comprise at least one winding or several windings around the lumen portion. Both alternatives may result in a good fixation of the wire on the lumen portion and/or a good connection of the wires to the cannula. Additional, fasting means may be used, e.g. adhesive etc. as is described below.

The connection region of a wire may also be named as proximal mounting portion that may comprise a circumferential portion. There may be a corresponding mounting portion of the expandable arrangement which comprises the mounting portions of several or of all of the wires.

At least one wire, at least two wires or all wires of the plurality of the wires may comprise a proximal mounting portion that comprises a circumferential portion in which the wire extends along a circular or oval curve, preferably along at least two thirds or at least three quarters of the circumference of a circle or of an ellipse but not along the complete circumference or preferably along at least on winding or at least two windings, and a straight or less bended portion that is preferably arranged outside a plane in which the circumferential portion is arranged. The combination of both portions may allow the usage of special connection techniques.

The straight portion or the less bended portion of at least one wire, of at least two wires or of all wires of the plurality of the wires may extend across at least one of the wires, or across some of the wires, preferably axially and/or across circumferential portions. This crossing of wires may be independent of the state of expandable arrangement. Furthermore, there may be mechanical, i.e. physical contact between the wires that are crossing.

The straight portions or the less bended portions of at least one wire, of at least two wires or of all wires of the plurality of wires may be connected to the circumferential portion of at least some other of the wires by welding or soldering or by using an adhesive in order to form a good connection between the wires, e.g. a self-supporting connection. However, alternatively or additionally further connection elements may be used, for instance a sleeve.

The wires may comprise a material that has a shape memory, preferably a shape memory that is depending on temperature or that is not depending or only slightly depending on temperature. The material of the wires may comprise or consist of Nitinol (may be a registered trade mark), titanium, titanium alloys or copper-aluminum-nickel alloys. Thus, the wires may have a pre-bended shape that corresponds to the shape in the expanded state. In the non-expanded state the pre-shaped wires may be stretched for instance by an introducer member that is inserted into the expandable arrangement or by sheath member that is wound around the expandable arrangement. A preferred material for the wires may be a shape memory alloy (SMA) or a shape memory material, for instance a material that changes its shape depending on the temperature of the material. Nitinol (Nickel Titanium Naval Ordnance Laboratory) is an example for such a material. However, other materials may also be used, for instance NiTi (nickel titan), NiTiCu (nickel titan copper), CuZn (copper zinc), CuZnAl (copper zinc aluminum) and/or CuAlNi (copper aluminum nickel). Further materials that may be used are super elastic materials, stainless steel wire, cobalt-chrome alloys or cobalt-chromium-nickel-molybdenum-iron alloy. The thickness and/or diameter of the wires may be in the range of 0.1 mm (millimetre) to 2 mm, especially if only three or four wires are used within the expandable arrangement that may also be named as cage arrangement. The thickness and/or dimeter of the wires may be in the range of 0.1 mm (milli meter) to 1 mm or in the range of 0.25 mm to 0.75 mm Thinner wires may be useful if more than four wires are comprised within the cage arrangement.

The expandable arrangement may comprise a proximal portion that is different from the connection or mounting portion. In the expanded state of the expandable arrangement, the distance between angularly adjacent or angularly neighboring wires in the proximal portion increases with increasing distance to a mounting portion of the wires on the cannula, e.g. the wires extend away from each other. The expandable arrangement may comprise a distal portion, wherein in the expanded state of the expandable arrangement the distance between angularly neighboring wires in the distal portion decreases with increasing distance to the mounting portion of the wires, e.g. the wires extend closer to each other with increasing distance to the mounting portion.

The expandable arrangement may comprise an optional transition portion, wherein in the expanded state of the expandable arrangement the distance between angularly neighboring wires in the optional transition portion is constant with increasing distance to the mounting portion of the wires. The transition portion may be appropriate for special application scenarios. An axial extension of the transition portion may be longer than an axial extension of the proximal portion in the expanded state and/or longer than an axial extension of the distal portion in the expanded state. Thus, the transition portion may be a main part of the expandable arrangement.

The expandable arrangement may comprise in the expanded state following a distal portion, a radially extending straight portion in which the wires extend only radially inward at the same axial position for at least 1 mm, at least 2 mm, at least 3 mm, at least 4 mm or at least 5 mm. The radially extending portion may be appropriate to make atraumatic contact with a wall of a vessel or with other portions of the body.

Furthermore, there may be a backwardly bended portion, preferably an inwardly bended portion. The backwardly bended portion may allow atraumatic insertion of the cannula, preferably endovascular insertion. Within the backwardly bended portion the wires may change direction and/or neighboring wires may have decreasing distances with decreasing distance to the mounting portions of the wires. The backwardly bended portion may be encompassed by portions of the wires of the expandable arrangement that do not belong to the backwardly bended portion. The expandable arrangement may comprise a cage tip portion following the backwardly bended portion. In the cage tip portion, the cage wires may be connected with each other. There may be a backward bending by more than 90 degrees, by more than 110 degrees, by more than 120 degrees, or more than 140 degrees up to for instance 180 degrees. Alternatively, the expandable arrangement may not comprise a backwardly bended portion.

At least one wire, at least three wires or all wires of the plurality of wires may comprise in the expanded state a mounting portion that comprises:
preferably a circumferential portion in which the wire may be bended circular or oval, for instance along at least three quarters of the circumference of a circle or of an ellipse but not along the complete circumference or preferably along at least on winding or at least two windings, and
preferably a straight portion or less bended portion that may be preferably arranged parallel to the extended longitudinal axis of the lumen portion,
a proximal portion in which the wire may have an increasing radial distance to the extended longitudinal axis of the lumen portion with increasing distance to the mounting portion,
an optional transition portion in which the wire has a constant radial distance to the extended longitudinal axis of the lumen portion with increasing distance to mounting portion,
a distal portion in which the wire has a decreasing radial distance to the extended longitudinal axis of the lumen portion with increasing distance to mounting portion, and
a backwardly bended wire portion, and
preferably a radially extending straight portion in which the wire extends only radially inward at the same axial position for at least 1 mm, at least 2 mm, at least 3 mm, at least 4 mm or at least 5 mm,
preferably a cage tip portion that may be covered by plastic cap and/or in which the wire is parallel to the extended longitudinal axis of the lumen portion.

Thus, the course of the wires may correspond to respective portions of the expandable arrangement.

Wires of the expandable arrangement of the cannula may be distributed angularly such that, in a given axial position preferably in the expanded state, for a first pair of angularly neighboring wires the wires forming the pair have a first distance relative to each other and that angularly neighboring wires forming a second pair of neighboring wires may have a second distance relative to each other that is greater than the first distance, preferably equal to or greater than twice the first distance. This, may mean that at least one wire is omitted or at least two adjacent wires are omitted in order to create a wider gap between two neighboring (angularly adjacent) wires compared to the gaps or maximal distances between other pairs of wires. There may also be two or more than two wider gaps, e.g. omitted wires. The wider gaps may be used to ease the passing of a medical devices, for instance a clip for a mitral valve etc., for an endoscope or for a second cannula.

The cage wires may be distributed angularly such that, in a given axial position, for two different pairs of neighboring cage wires the wires forming the pair have respectively a first distance relative to each other which is equal for both pairs and that neighboring wires forming yet another pair of neighboring wires have a second distance relative to each other that is greater than the first distance, preferably equal to or greater than twice the first distance.

The expandable arrangement may comprise at least one membrane. The membrane may be folded or less stretched in the non-expanded state and may be expanded in the expanded state of the expandable arrangement. The membrane may be connected to at least two of the plurality of wires. The membrane may limit the inner volume of expandable arrangement, especially in the expanded state. The membrane may be used to guide a fluid that is delivered into the body or drained out of the body using the cannula. The membrane may have further or alternative functions, e.g. at least one, at least two, at least three or all of the following functions:
isolating fluid flows, for instance the natural blood flow from the fluid flow that flows also through the cannula, and/or
guiding the fluid flow that flows out of the cannula into a specific direction, and/or
preventing the “sand blasting” effect, e.g. damage of a wall of vessel by a fluid flow that flows through the cannula into the body of the subject, and/or
collecting a fluid flow that flows into the cannula, e.g. same as a funnel, and/or
directing medicaments and/or toxic treatment fluids into the relevant parts of the body but preventing that the medicament and/or toxic treatment fluid gets to parts of the bodies where it would do more harm compared to its healing effect.

Additionally or alternatively, the membrane may define a volume which is fluidly connected to the lumen portion but with a greater diameter than the lumen portion, especially in the expanded state of the expandable arrangement. The membrane may be fluid tight and the opening may be an inlet into the lumen portion or an outlet out of the lumen portion.

The membrane may be folded or less stretched in the non-expanded state of the expandable arrangement, e.g. it may have lots of wrinkles. The wrinkles may be arranged between supporting structures or outside of spaces that are arranged between such supporting structures, e.g. wires. The first membrane may be expanded in the expanded state of the expandable arrangement. The membrane may be connected to the expandable (diameter variable) arrangement using several techniques, for instance dip molding, plastic welding, sewing and/or using glue or adhesive. The connection technique may also make a fluid tight connection to the cannula or to a mounting part of the diameter variable arrangement on the cannula.

The material of the membrane may be liquid-tight (impermeable) in both directions, i.e. from inside of diameter variable arrangement (cage) to the surrounding area and/or from surrounding area to inside of cage. However, semi-permeable membranes may also be used if necessary and appropriate.

The membrane may be or comprise a thin sheet of material having for instance a thickness of less than 0.5 millimeters, less than 100 micrometer or less than 50 micrometer (micron). However, the membrane may be thicker than 10 micrometer in order to provide appropriate strength. Polytetrafluoroethylene (PTFE) may be an appropriate material for the membrane, preferably if it is manufactured by electro spinning, for instance within an electrical field. However, other materials may also be used. Only one sheet of membrane material may be arranged for forming the membrane, e.g. there is only one layer of membrane material.

In the expanded state, the membrane may define:
an opening that faces distally relative to the longitudinal axis of the lumen portion of the cannula, and/or
an opening that faces laterally relative to the cannula, and/or
an opening that faces proximally relative to the longitudinal axis of the lumen portion of the cannula, e.g. in applications for treatment of the kidney, renal artery etc.

The edge of the membrane having the distally facing opening may delimit the edge of the opening along the entire circumference of the opening. Thus, the membrane may extend circumferential around the extended longitudinal axis of the lumen portion by at least 360 degrees or by 360 degrees.

The membrane having the distally facing opening may extend from a proximal end of the expandable arrangement at most three quarters or at most half way or at most one quarter to a distal end of the expandable arrangement. This means that at least one quarter of the axial length of the expandable arrangement may not be covered by the membrane having the distally facing opening, preferably a proximal part of the expandable arrangement.

The edge of the membrane comprising the distally facing opening may extend transversally to cage wires of the expandable arrangement. A membrane having a distally facing opening may have, especially in the expanded state, the following relations relative to portions of the expandable arrangement that are mentioned above:
the proximal portion and/or the optional transition portions or parts thereof may be covered by the membrane, and
the distal portion and/or the optional transition portion or parts thereof may not be covered by the membrane.

An edge of the membrane that comprises an opening that faces laterally may delimit the edge of the opening along the entire circumference of the opening. The membrane having an opening facing laterally may extend circumferential around the extension of the longitudinal axis of the cannula by less than 270 degrees or by less than 200 degrees and may extend from a proximal end of the expandable arrangement to a distal end of the expandable arrangement or along at least 80 percent of this axial distance. The membrane may cover the expandable arrangement from 0 degree to 180 degrees or from 0 degree to 210 degrees or from 0 degree to 240 degrees.

A membrane having a laterally facing opening may have, especially in the expanded state, the following relations relative to the portions of the expandable arrangement that are mentioned above:
the proximal portion, the optional transition portion, and the distal portion may be covered by the membrane on a first side of the expandable arrangement, and
the proximal portion, the optional transition portion and the distal portion may not be covered by the membrane on a second side of the expandable arrangement that is preferably opposite to the first side of the expandable arrangement.

In both cases, i.e. membrane comprising a distally facing opening and membrane comprising a laterally facing opening, the volume that is defined by the membrane may be an extension of the inner lumen of the lumen portion, i.e. it may extend the lumen for a fluid that flows out of the cannula or it may narrow the lumen for a fluid that flows into the cannula, e.g. same as a funnel. The edge of the membrane comprising the laterally facing opening may extend parallel to cage wires of the expandable arrangement.

The cannula may be adapted to be inserted endovascular into the heart, and preferably further into the aorta or into the pulmonary artery. Thus, minimal invasive surgery may be used.

The cannula may not extend within the expandable arrangement or may maximally extend into the expandable arrangement by at most 10 mm or at most 5 mm One end-hole cannula may be advantage for:
High flow rates, and/or
Less wall shear stress, and/or
Less shear rates, and/or
Less turbulences, and/or
Less recirculation, and/or
More homogeneous velocity profile.

Negative effects of the single end-hole cannula may not arise due to the presence of the cage arrangement and/or of the membrane that is coupled to the cage arrangement. There may also be no tip of the cannula within the expandable arrangement. Additionally or alternatively, the cannula may comprise at least one end hole or a single end-hole through which at least 25 volume percent, at least 50 volume percent, at least 75 volume percent or at least 90 volume percent or all of the flow flows into or out of the cannula. The volume percent of the flow may be measured at an overall flow through the cannula of for instance 3.5 liter per minute or 5 liter per minute.

Furthermore, a cannula system is disclosed that comprises a cannula according to one of the embodiments mentioned above. The cannula that is mentioned above may be a first cannula. The cannula system may comprise a second cannula that may be arranged at least partially within the first cannula and/or that may be adapted to be inserted into and/or through the first cannula or that is adapted such that the first cannula is arranged within the second cannula or such that the first cannula can be inserted into and/or through the second cannula. This means that a fixed multi-lumen cannula system or a fixed dual lumen cannula system may be used, e.g. having cannulas that are not axially movable relative to each other or only to a less extend of for instance less than 5 cm (centimeter), especially during use or introduction into the body of the subject. A sheath member may be used to hold the expandable arrangement of outer cannula in the non-expanded state if fixed dual cannula system is used.

Alternatively, a not-fixed (insertable) multi-lumen cannula system or a not-fixed dual lumen cannula system may be used, e.g. having cannulas that are axially movable relative to each other, especially during use or introduction into the body of the subject. One cannula is introduced first into the body and the other cannula is introduced thereafter through the cannula that is already within the body, preferably distally out of the other cannula. Not-fixed multi-lumen cannula systems are less traumatic compared to fixed multi-lumen cannula as the stiffness and/or friction during insertion may be reduced considerably. A first introducer member may be used for introducing the first cannula and/or for stretching the expandable arrangement. A second introducer member may be used for introducing the second cannula of the not-fixed multi-lumen cannula system.

The second cannula may comprise flexible further lumen portion that extends axially between a proximal part of the second cannula and a distal part of the second cannula. At least one distal part of the further lumen portion may carry a further expandable arrangement.

The expandable arrangement of the second cannula may be adapted to have an expanded state in which a volume defined by the expandable arrangement of the second cannula is greater than the volume defined by the expandable arrangement of the second cannula in a non-expanded state. The same technical effects may be valid as for the expandable arrangement of the first cannula.

Wires of the expandable arrangement of the first cannula may be distributed angularly such that, in a given axial position preferably in the expanded state, for a first pair of angularly neighboring wires the wires forming the pair have a first distance relative to each other and that angularly neighboring wires forming a second pair of neighboring wires have a second distance relative to each other that is greater than the first distance, preferably equal to or greater than twice the first distance. The second cannula may be extended through or be insertable through the wider gap within the cage arrangement. The omission of one or of more wires, especially of two or more adjacent wires may ease the insertion of the second cannula through the expandable arrangement of the first cannula. Further, the non-expanded state may have a smaller maximal diameter if at least one wire is omitted.

Furthermore, the expandable arrangement of the first cannula may comprise a membrane. The membrane may be folded or less stretched in the non-expanded state of the expandable arrangement of the first cannula and wherein the membrane is expanded in the expanded state of the expandable arrangement of the first cannula. A segment of the membrane may be arranged between the wires of the expandable arrangement of the first cannula, preferably between the wires of the second pair. The segment of the membrane may have an insertion opening that is adapted to the diameter of the second cannula. The insertion opening may be adapted such that the second cannula is insertable or is inserted through the insertion opening thereby closing at least the main part or all of the insertion opening.

The first cannula may not extend within the expandable arrangement of the first cannula or may maximally extend into the expandable arrangement of the first cannula by at most 10 mm or at most 5 mm or at most 3 mm Thus, a single end-hole cannula may be formed that allows for instance:
High flow rates, and/or
Less wall shear stress, and/or
Less shear rates, and/or
Less turbulences, and/or
Less recirculation, and/or
More homogeneous velocity profile.

There may be no additional tip of the first cannula within the expandable arrangement. Additionally or alternatively, the first cannula may comprise at least one end-hole or a single end-hole through which at least 25 volume percent, at least 50 volume percent, at least 75 volume percent or at least 90 volume percent or all of the flow flows into or out of the cannula. The volume percent of the flow may be measured at an overall flow through the cannula of for instance 3.5 liter per minute or 5 liter per minute.

The cannula system may comprise a further cannula according to one of the embodiments mentioned above. The further cannula may be a third cannula. Moreover, the cannula system may comprise a fourth cannula that is arranged at least partially within the third cannula or that is adapted to be inserted into and/or through the third cannula or that is arranged within the fourth cannula or that is adapted such that the third cannula can be inserted into and/or through the fourth cannula. Thus, two multi lumen systems may be used at the same time, preferable within the heart of a subject. Fixed and not-fixed multi-lumen systems may be combined or only one kind of multi-lumen systems may be used, i.e. only fixed multi-lumen systems or only not-fixed multi-lumen systems comprising a cannula that is insertable into the other cannula of the same multi-lumen system during use. A broad range of new applications may be opened in the medical and/or non-medical field.

The fourth cannula may carry an expandable arrangement that is adapted to have an expanded state and a non-expanded state. This may further extend the range of new applications. A sheath may be used to hold the expandable arrangement of the outer cannula in the non-expanded state if a fixed dual-lumen cannula system or multi-lumen cannula system is used and if for instance wires are used within the expandable arrangement of the outer cannula. Alternatively, an inflatable balloon may be used to realize a fixation function for the outer cannula.

Furthermore, a method for inserting a cannula into a subject is disclosed, e.g. for cannulization or cannulizing a subject. The method may comprise:
providing a cannula that comprises at least one lumen portion and an expandable arrangement at least one a distal part of the at least one lumen portion, and/or
inserting the cannula into a body of the subject whereby the at least one expandable arrangement is in a non-expanded state, and/or
expanding the at least one expandable arrangement, for instance a cage arrangement, a balloon or another arrangement, thereby securing the cannula within the body, and/or
using a lumen within the at least one lumen portion to guide a fluid into the body and/or out of the body. The same technical effects may apply that are mentioned above for the cannula and/or the cannula system.

The method may further comprise:
inserting an introducer member into the cannula in order to bring the expandable arrangement into the non-expanded state in which the expandable arrangement defines a first volume, and/or
inserting the cannula into the body of the subject using the introducer member until the distal end of the cannula reaches a final destination place, and/or
thereafter pulling back the introducer member leaving the expandable arrangement in the expanded state that defines a second volume that is greater than the first volume, preferably at least twice the first volume, and/or
removing the introducer member out of the cannula before the fluid is guided into the body and/or out of the body.

Usage of an introducer member is simple but effective for cannulas, especially for cannulas having an expandable cage arrangement with a tip portion that forms a contact portion for the introducer member, e.g. the tip portion is opposite an end-hole of the cannula.

The expandable arrangement may be connected to the at least one lumen portion using a connection technique that connects two different separate parts of the same or of different material. One of the following techniques may be used: welding, soldering, using an adhesive and/or winding around or along a surface, especially of the lumen portion or of a sleeve or of another mounting member.

The expandable arrangement may comprise a plurality of wires, preferably comprising or consisting of a metal. Compared to the usage of inflatable balloon it is not necessary to have auxiliary fluid conduits from the proximal end to the expandable arrangement. Furthermore, no additional source is necessary to provide the auxiliary fluid.

Proximal ends of at least two of the wires or of all of the wires may be connected to a distal end of the lumen portion. Distal ends of at least two wires, of at least three of the plurality of wires or of all wires of the plurality of the wires are preferably connected with each other and/or with a connection element, preferably at a position that is on the extended longitudinal axis of the lumen portion of the first cannula. The wires may be twisted with each other and or the distal parts of the wires may be arranged parallel or essentially parallel to the longitudinal axis of the lumen portion. Thus, an expandable cage arrangement may be realized in a simple manner.

The wires of the expandable arrangement of the cannula may be distributed angularly such that, in a given axial position in the expanded state the following is valid. A first pair of angularly adjacent or angularly neighboring wires may form a pair that has a first distance relative to each other, for instance a maximal distance, and angularly adjacent or angularly neighboring wires that may form a second pair of neighboring wires may have at the same given axial position a second distance relative to each other, for instance a maximal distance, that is greater than the first distance, preferably equal to or greater than twice the first distance. This may mean that at least one wire or at least two angularly adjacent wires may be omitted to form a wider gap between two angularly adjacent wires. The wider gap may be used to allow or ease the insertion of at least one medical device, for instance a clip for mitral valve etc., of an endoscope, of a further cannula. The wider gap may have other functions or further functions.

The expandable arrangement may comprise a membrane. The membrane may be folded or less stretched in the non-expanded state of the expandable arrangement and the membrane may be expanded in the expanded state of the expandable arrangement. The membrane may be impermeable for the fluid that is delivered into the body or drained out of the body using the cannula. However, there may be applications for permeable or semipermeable membranes, i.e. in one direction permeable and in the other direction impermeable. Further characteristics of the membrane are mentioned above.

In its expanded state the membrane may define:
an opening that faces distally relative to a longitudinal axis of the lumen portion of the cannula, or
an opening that faces laterally relative to the longitudinal axis of the lumen portion of the first cannula, or
an opening that faces proximally relative to the longitudinal axis of the lumen portion of the first cannula. This may be appropriate for medical applications that relate to the kidney, especially to the renal artery.

Application examples for the first two alternatives are described below.

A segment of the first membrane may be arranged between wires of the expandable arrangement, preferably between the wires of the second pair of wires as far as referred back to the embodiment where at least one wire of the cage arrangement is omitted.

The segment of the first membrane may have an insertion opening that is adapted to the diameter of a second cannula. The second cannula may close the main part of the insertion opening or is inserted through the insertion opening thereby closing the main part of the insertion opening. Thus, applications are possible using a dual-lumen cannula system or a multi-lumen cannula system comprising expandable arrangements and preferably also membranes that are supported by the expandable arrangement, especially by expandable cage arrangements.

The cannula may be inserted endovascular to or through at least one chamber of the heart. The expandable arrangement may be placed within the heart of the subject, preferably in the left atrium or in the left ventricle of the heart, preferably atrial trans septal or ventricle trans septal, or within the right atrium or within the right ventricle. Thus, depending on the application all parts of the heart may be reached. The expandable arrangement may form a fixation member for the cannula in this application scenario.

The cannula may be inserted endovascular to or through at least one chamber of the heart and the expandable arrangement may be placed within the aorta of the subject, preferably in the ascending aorta. There may be application that deliver oxygenized blood into the aorta through the cannula. The expandable arrangement may form a fixation member for the cannula in this application scenario.

Alternatively, the cannula may be inserted endovascular to or through at least one chamber of the heart and the expandable arrangement may be placed within the pulmonary artery of the subject, preferably within the common pulmonary artery, within the right pulmonary artery or within the left pulmonary artery. The cannula may drain blood from the pulmonary artery or blood may be delivered into the pulmonary artery. Medicaments and/or chemistry for treating cancer may be added to a fluid/blood in order to treat a lung disease. The expandable arrangement may form a fixation member for the cannula in this application scenario.

The cannula may be inserted endovascular femoral and the expandable arrangement may be placed within the common femoral artery, within the thoracic aorta or within the abdominal aorta, preferably transcaval. The expandable arrangement may form a fixation member for the cannula in this application scenario. Known transcaval connection elements may be used that remain within the body after removal of the cannula and may be used several times.

The cannula may be inserted endovascular jugular venous, through vena cava or femoral venous and then transcaval into the descending thoracic aorta or within the abdominal aorta. A jugular access allows mobility of the patient. Mobility may be important if the cannula remains within the body for a longer time, for instance for more than one day, for more than two days, for more than one week, for more than two weeks etc.

The cannula may be a first cannula and the method may comprise:
providing a second cannula,
wherein the second cannula is arranged within the first cannula or wherein the second cannula is inserted into the first cannula after the first cannula is within the body or wherein the first cannula is arranged within the second cannula or wherein the second cannula is inserted into the body before the first cannula is inserted through the second cannula into the body.

Thus a multi-lumen cannula system may be used. The technical effects and variants, e.g. fixed and non-fixed are described above.

The second cannula may comprise a flexible lumen portion that extends axially between a proximal part of the second cannula and a distal part of the second cannula. The second cannula may carry a second expandable arrangement, preferably an expandable arrangement that comprises wires.

The second cannula may be inserted using an introducer member. The introducer member may be used to bring an expandable arrangement on a distal part of the second cannula into the non-expanded state during insertion of the second cannula. The expandable arrangement may self-expand to the expanded state if the introducer member is removed from a contact area of the expandable arrangement and further out of the second cannula.

The second expandable arrangement may comprise a membrane. The membrane of the second expandable arrangement may be folded or less stretched in the non-expanded state of the expandable arrangement of the second expandable arrangement. The membrane of the second expandable arrangement may be expanded in the expanded state of the second expandable arrangement. This opens a broad range of medical application scenarios.

In its expanded state, the membrane of the second expandable arrangement defines an opening that faces distally relative to a longitudinal axis of the second cannula or that faces transversally relative to the longitudinal axis of the second cannula or that faces proximally relative to the longitudinal axis of the second cannula. Reference is made to the description above, e.g. the same features and/or technical effects are valid that are given for openings of the membrane at the first cannula.

The first cannula may be inserted endovascular through the right atrium, the atrial septum into the left atrium where the first cannula is fixed by the expandable arrangement of the first cannula. The second cannula may be inserted through the first cannula and the expandable arrangement of the first cannula into the left ventricle and then into the aorta, preferably into the ascending aorta, where the second cannula is fixed by the second expandable arrangement. Thus, a simple solution for a pLVAD (percutaneous left ventricle assisted device) method is disclosed. This medical application may be combined with other medical applications as is described below, especially with applications involving lung assist or lung treatment.

The first cannula may be inserted endovascular through the superior vena cava into the right atrium of the heart or into the right ventricle where preferably the first cannula is fixed by the expandable arrangement of the first cannula. The second cannula may be inserted through the first cannula and the optional expandable arrangement of the first cannula into the common pulmonary artery, into the left pulmonary artery or into the right pulmonary artery where the second cannula is fixed by the expandable arrangement of the second cannula. Thus, a simple solution for lung assist or lung treatment medical applications is proposed. This medical application may be combined with other medical applications as is described below, especially with applications involving blood delivery into the aorta.

Further to the application involving a pulmonary artery, the first cannula may comprise a first group of holes that is arranged in the right ventricle and/or the first cannula may comprise a second group of holes that is arranged within the right atrium. The first group of holes and/or the second group of holes may be used for drainage of fluid or blood from the heart, i.e. for right heart relief. Thus, a multi stage drainage application is given that allows high flow rates of drainage flow, for instance more than 1 liter per minute, more than 2 liters per minute or more than 3 liters per minute.

A portion of at least 1 cm, at least 1.5 cm or of at least 2 cm axial length and without holes may be arranged between the first group of holes and the second group of holes. Thus, both groups are clearly separated. The tricuspid valve may be arranged near the portion that does not have drainage holes in order to avoid detrimental turbulences.

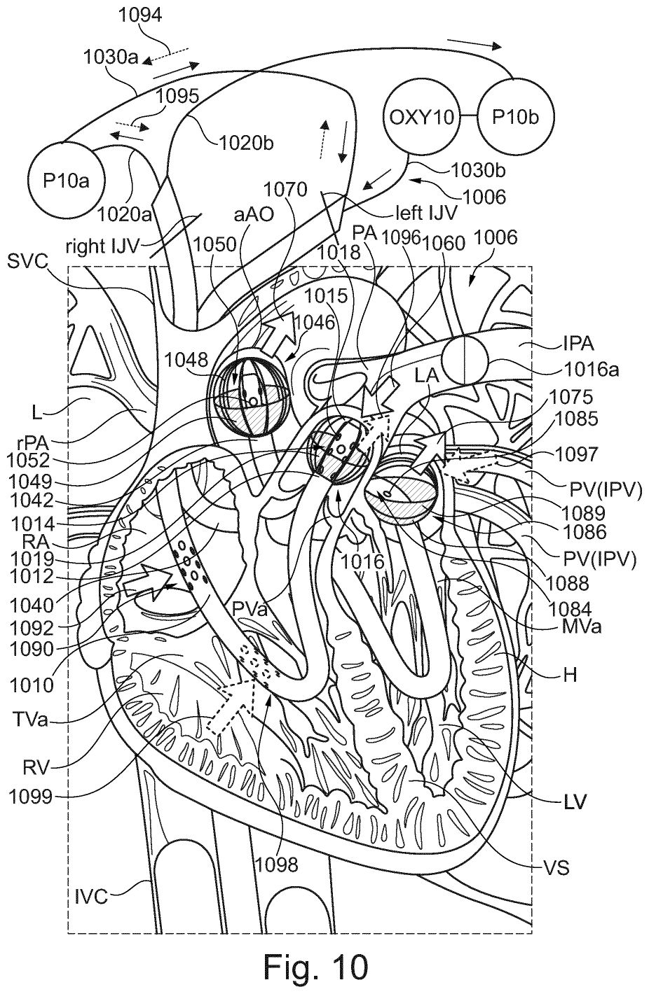

A third cannula may be inserted endovascular through the right atrium, the atrial septum into the left atrium where the third cannula is fixed by an expandable arrangement of the third cannula. The fourth cannula may be inserted through the third cannula and the expandable arrangement of the third cannula into the left ventricle and then into the aorta, preferably into the ascending aorta, where the fourth cannula may be fixed by an expandable arrangement of the fourth cannula. This medical application is described below in more detail, see also FIG. 10.

At least one sealing element may be arranged within an opening through which the second cannula is inserted into the first cannula, e.g. at a proximal end of the first cannula. The sealing element may comprise a retaining ring, a sealing ring, a gasket or a multi-flap valve or another self-sealing element. Two membranes may be used that have openings having a lateral offset if the membranes are flat. The openings may be aligned by inserting the second cannula thereby stretching the membranes. Alternatively, only one membrane having a small hole or no hole may be used that is pierced by the second cannula or by an introducer.

Additionally or alternatively, a closure element may be arranged at the distal part of the first cannula that prevents the passage of fluid through the distal end of the first cannula into the first lumen of the first cannula and/or vice versa. The closure element may allow the passage of the second cannula and/or of an introducer member. The closure element may comprise a multi-flap valve or another self-sealing element, for instance two membranes comprising holes with lateral offsets or only one membrane as mentioned above.

The first fluid and/or the second fluid may be injected into the body and/or taken out of the body in a pulsed fluid flow or in a continuous fluid flow. Thus, an extracorporeal method is disclosed. A pulsed fluid flow corresponds to the pulsation of the natural fluid flow and may be more appropriate for the body, for instance in order to reduce the risk of the formation of thrombus, as the blood flow is likely less laminar and more turbulent. However, flow rates may be too low if a pulsatile flow is used. Roller pumps may create a pulsatile flow rate in a simple manner, e.g. pressing a tube in a periodic manner. However, centrifugal pumps may be used if a periodic control method is used.

A continuous flow may allow higher flow rates. A centrifugal pump may be used to realize a continuous fluid flow.

Furthermore, some of the applications may not involve the usage of a pump, i.e. a pumpless solution may be appropriate if the blood pressure difference within the body is sufficient to create the fluid flow within the cannula or within the cannulas.

The first cannula and/or the second cannula may be pre-bended or both cannulas may be pre-bended by an angle within the range of 60 degrees to 175 degrees or of 70 degrees to 145 degrees, preferably in order to ease an insertion through the septum of the heart of the subject, preferably through the atrial septum or through the ventricle septum. Alternatively, insertion through the right ventricle may be eased by pre-bending.

A cannula according to one of embodiments mentioned above may be used for the method and its embodiments. A cannula system according to one of the embodiments mentioned above may be used for the method or its embodiments. Furthermore, features of the cannulas that are used in the method may be claimed for the cannula or the cannula system itself, for instance the feature that relates to the bending of the cannula.

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

The term “haemoperfusion/hemoperfusion”, as used herein, refers to a method of filtering blood extracorporeally (that is, outside the body) to remove one or more toxins. As with other extracorporeal methods, such as hemodialysis (HD), hemofiltration (HF), and hemodiafiltration (HDF), the blood travels from the patient into a machine, gets filtered, and then travels back into the patient, typically by venovenous access (out of a vein and back into a vein). During the extracorporeal process, inflammatory and other harmful molecules are removed from the blood of the organism of a patient. Adsorbers (filters), in particular macroporous resin beads, are preferably used, which allow an effective blood purification. These adsorbers in turn may be combined with other therapy components, which regulate the metabolism and strengthen the immune system. In this way, drug strategies can take effect more effectively. Alternatively and/or additionally absorber techniques may be used. However, the filtering may alternatively concern a fluid flow that does not contain blood or that does not contain blood as the main part, i.e. it contains blood or blood components only by 50 percent per volume or less than 50 volume percent.

The term “hyperoxygenated haemoperfusion/hemoperfusion”, as used herein, refers to the haemoperfusion/hemoperfusion as described above but extended by an oxygenator that generates an oxygen level in the blood or in the fluid flow that is used for treatment that is higher than a normal oxygen level within blood, for instance if the body rests. This the oxygen content of the blood may be increased, for instance at least by 5 percent or at least by 10 percent. The oxygen content of body tissue may be increased even more, for instance by more than 10 percent or more than 50 percent compared for instance to a normal oxygen level, for instance if the body rests. Hyperoxygenation may have a positive effect for the uptake of medicaments and/or treatment substances by the body or more specific by the organ that is under treatment.

The term “hypooxygenated haemoperfusion/hemoperfusion”, as used herein, refers to the haemoperfusion/hemoperfusion as described above but extended by an oxygenator that generates an oxygen level that is lower than the normal oxygen level within blood, for instance if the body rests. This may reduce the oxygen content of the blood and/or of body tissue, for instance by more than 10 percent or by more than 50 percent. Hypooxygenation may have a positive effect for the uptake of medicaments and/or treatment substances by the body or more specific by the organ that is under treatment. Furthermore, there may be medicaments and/or treatment substances that react with oxygen. This reaction may be detrimental for the therapeutic effect and it may be advantageous to prevent or reduce the reaction as far as possible.

The term “disease”, as used herein, refers to an abnormal condition that affects the body of an individual. A disease is often construed as a medical condition associated with specific symptoms and signs. A disease may be caused by factors originally from an external source, such as infectious disease, or it may be caused by internal dysfunctions, such as autoimmune disease. In humans, “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infectious, isolated symptoms, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. In the context of the present invention, the disease may be selected from the group consisting of cancer and infectious disease.

The terms “cancer disease” or “cancer”, as used herein, refer to or describe the physiological condition in an individual that is typically characterized by unregulated cell growth. Examples of cancers include, but are not limited to, carcinoma, lymphoma, blastoma and sarcoma. More particularly, examples of such cancers include lung cancer, liver cancer or cancer of other organs.

The terms “individual” and “subject” can be used interchangeable herein. The individual or subject may be any mammal, including both a human and another mammal, e.g. an animal. Human individuals or subjects are particularly preferred. The individual may be a patient.

The term “patient”, as used herein, refers to any subject suffering from a disease, in particular suffering from cancer, an autoimmune disease, and/or infectious disease. The patient may be treated and/or the response to said treatment may be evaluated. The patient may be any mammal, including both a human and another mammal, e.g. an animal Human subjects as patients are particularly preferred.

The term “treatment”, in particular “therapeutic treatment”, as used herein, refers to any therapy which improves the health status and/or prolongs (increases) the lifespan of a patient. Said therapy may eliminate the disease in a patient, arrest or slow the development of a disease in a patient, inhibit or slow the development of a disease in a patient, decrease the frequency or severity of symptoms in a patient, and/or decrease the recurrence in a patient who currently has or who previously has had a disease.

A drug used in chemotherapy is a chemotherapeutic agent. The term “chemotherapeutic agent”, as used herein, refers to a compound that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. The chemotherapeutic agent is preferably selected from the group consisting of alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, retinoids and vinca alkaloids and its derivatives. Liquid drugs may be used.

However, usage of small balls or beads or nano particles or micro particles may be advantageous to deliver the medicament and/or the therapeutic substance, for instance usage of nanoballs Liposomes may be used to produce the nanoparticles or the micro particles.

The term “radiation therapy (also called radiotherapy)”, as used herein, refers to a cancer treatment that uses high doses of radiation to kill cancer cells and shrink tumors. At low doses, radiation is used in X-rays to see inside the body. At high doses, radiation therapy kills cancer cells or slows their growth by damaging their DNA. Cancer cells whose DNA is damaged beyond repair stop dividing or die. When the damaged cells die, they are broken down and removed from the body. Radiation therapy may not kill cancer cells right away. It may take days or weeks of treatment before DNA may be damaged enough for cancer cells to die. Then, cancer cells may keep dying for weeks or months after radiation therapy ends. Classical radiation by using ionizing radiation (high energy protons, electrons, neutrons, photons, particles) or by electronic X-ray devices may be used. Alternatively, radioactive substances may be brought into contact with the body, specifically with the treated organ or with a part of treated organ.

However, usage of small balls or beads or nano particles or micro particles may be advantageous to bring radioactive substances into the body, for instance usage of nanoballs.

The term “extracorporeal blood”, as used herein, refers to blood removed/isolated from an individual's blood circulation.

The term “extracorporeal circuit”, as used herein, refers to a procedure in which blood is taken from an individual's circulation to have a process applied to it before it is returned to the circulation. All of the system carrying the blood outside the body is termed the extracorporeal circuit.

The term “systemic administration”, a used herein, refers to the administration of the therapeutic agent, e.g. chemotherapeutic agent, such that said agent becomes widely distributed in the body of a patient in significant amounts and develops a biological effect. Typical systemic routes of administration include administration by introducing the therapeutic agent, e.g. chemotherapeutic agent, directly into the vascular system or oral, pulmonary, or intramuscular administration wherein the therapeutic agent, e.g. the chemotherapeutic agent, enters the vascular system and is carried to one or more desired site(s) of action via the blood. The systemic administration may be by parenteral administration. However, the proposed cannulas and methods may be especially advantageous for more local treatment of organs or of parts of organs.

The term “parenteral administration”, as used herein, refers to the administration of the therapeutic agent, e.g. chemotherapeutic agent, such that said compound does not pass the intestine. The term “parenteral administration” includes intravascular administration, intravenous administration, subcutaneous administration, intradermal administration, or intraarterial administration, but is not limited thereto.

It is also preferred that the extracorporeal blood is oxygenated blood. Preferably, the extracorporeal blood has been oxygenated by external means, e.g. using an oxygenator. The oxygenator enhances oxygen within the blood. Alternatively, the oxygen content of blood may be reduced below a normal level. However, normal oxygen levels within blood may be used as well.

It is further preferred that the blood is purified blood. Extracorporeal blood purification (EBP) is a treatment in which a patient's/donor's blood is passed through a device (e.g. membrane, sorbent) in which solute (e.g. waste products, toxins) and possibly also water are removed. When fluid is removed, replacement fluid is usually added. It is preferred that purified blood does not comprise inflammatory, toxic molecules and/or other harmful molecules anymore or comprises a reduced amount of said molecules compared to unpurified blood. Extracorporeal therapies designed to remove or filter substances from the circulation in order to purify blood include hemodialysis, hemofiltration, hemoadsorption, plasma filtration, cell-based therapies and combinations of any of the above. Preferably, the purified blood is filtered blood. More preferably, the purified blood is dialyzed blood. Blood dialysis removes waste, salt, toxins, extra water to prevent them from building up in the body, keeps a safe level of certain chemicals in the blood such as potassium, sodium, and bicarbonate, and helps to control blood pressure. It is particularly preferred that the blood, e.g. the purified, filtered, or dialyzed blood, is free of inflammatory or other harmful molecules.

It is further preferred that the cancer is selected from the group consisting of, lung cancer, urothelial cancer, bladder cancer, liver cancer, kidney cancer/renal cancer, stomach cancer.

It is further preferred that the chemotherapeutic agent is selected from the group consisting of alkylating agents, anthracyclines, cytoskeletal disruptors (taxanes), epothilones, histone deacetylase inhibitors, inhibitors of topoisomerase I, inhibitors of topoisomerase II, kinase inhibitors, nucleotide analogs and precursor analogs, peptide antibiotics, platinum-based agents, retinoids vinca alkaloids and its derivatives.

Systemic routes of administration may include administration by introducing the chemotherapeutic agent directly into the vascular system or pulmonary wherein the chemotherapeutic agent enters the vascular system and is carried to one or more desired site(s) of action. This is described in more detail below.

In particular, the chemotherapeutic agent may be suitable to be administered topically, intravenously, intraarterially, intrapleurally, by inhalation, via a catheter. The dose which can be administered to a patient (“a therapeutically effective amount” or simply “an effective amount”) should be sufficient to effect a beneficial therapeutic response in the patient over time. The dose will be determined by the efficacy of the particular chemotherapeutic agent employed and the condition of the patient, as well as the body weight or surface area of the patient to be treated. The size of the dose also will be determined by the existence, nature, and extent of any adverse side-effects that accompany the administration of the chemotherapeutic agent in a particular patient. The proposed invention may reduce adverse side-effects tremendously as the chemotherapeutic agent may be applied only locally and isolated from other body fluid circuits, especially form the blood circulation circuit of the body.

The chemotherapeutic agent may be administered in higher doses or in much higher doses compared to a systemic administration.

The proposed method and its embodiments may not be used for treatment of the human or animal body by surgery or therapy and may not be a diagnostic method practiced on the human or animal body. Alternatively, the proposed method and its embodiments may be used for treatment of the human or animal body by surgery or therapy and may be a diagnostic method practiced on the human or animal body.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference signs refer to the same technical features if not stated otherwise. As far as “may” is used in this application it means the possibility of doing so as well as the actual technical implementation. The present concepts of the present disclosure will be described with respect to preferred embodiments below in a more specific context namely heart and lung surgery. The disclosed concepts may also be applied, however, to other situations and/or arrangements in heart and lung surgery as well, especially to surgery of other organs.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

For a more complete understanding of the presently disclosed concepts and the advantages thereof, reference is now made to the following description in conjunction with the accompanying drawings. The drawings are not drawn to scale. In the drawings the following is shown in:

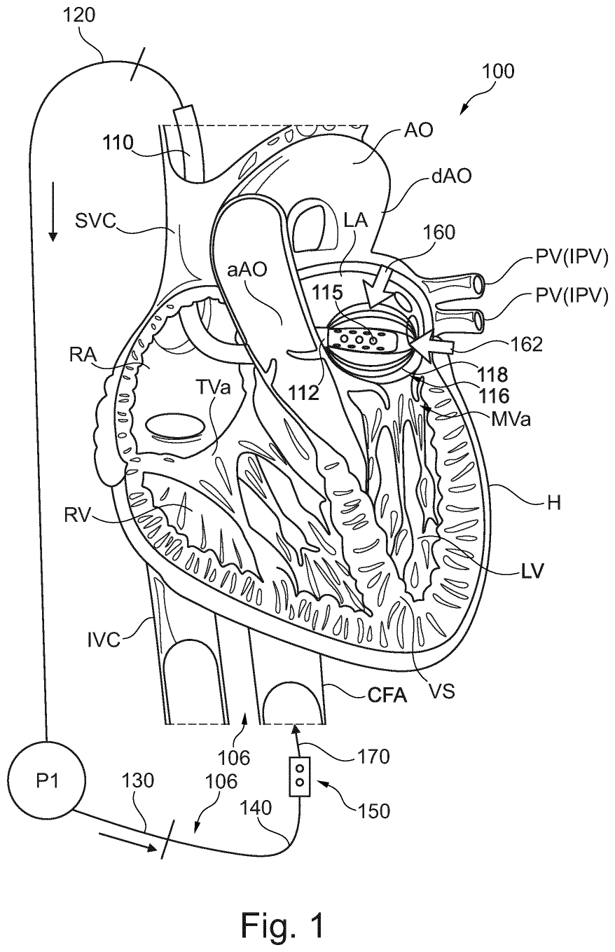

FIG. 1 an extra corporeal blood flow circuitry comprising two single lumen cannulas,

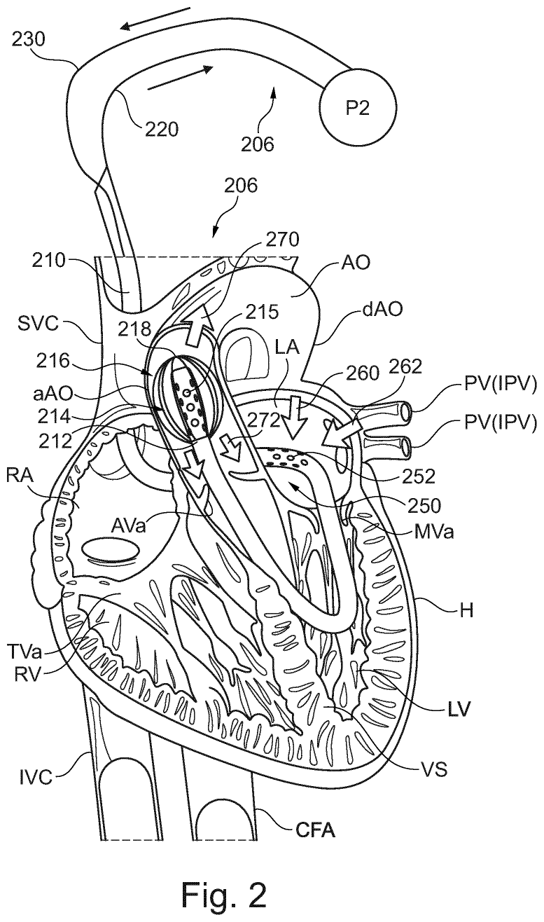

FIG. 2 an extra corporeal blood flow circuitry comprising a dual lumen cannula,

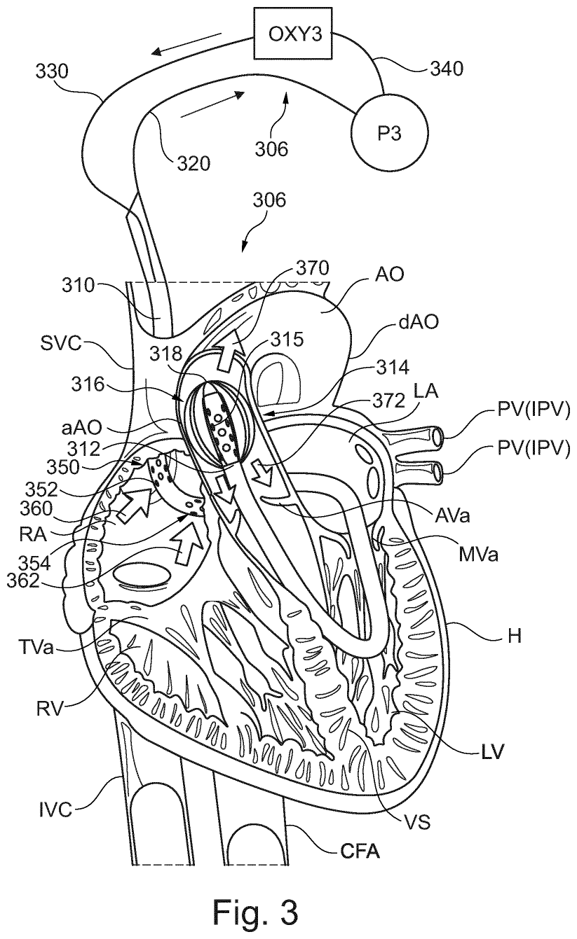

FIG. 3 an extra corporeal blood flow circuitry comprising a dual lumen cannula, a blood pump and oxygenator,

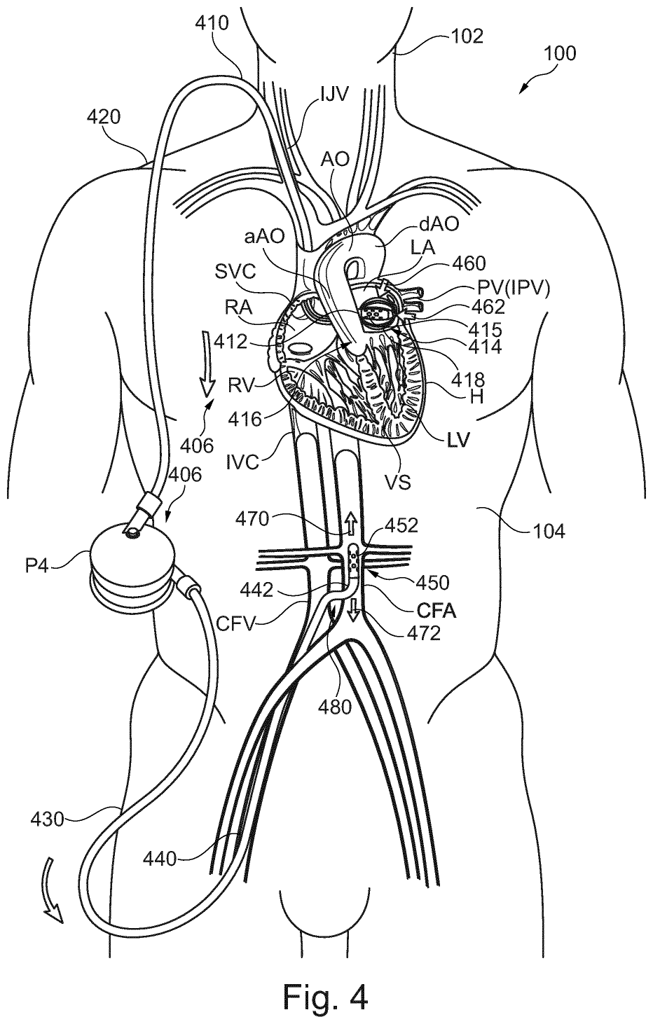

FIG. 4 a transcaval extra corporeal blood flow circuitry comprising two single lumen cannulas,

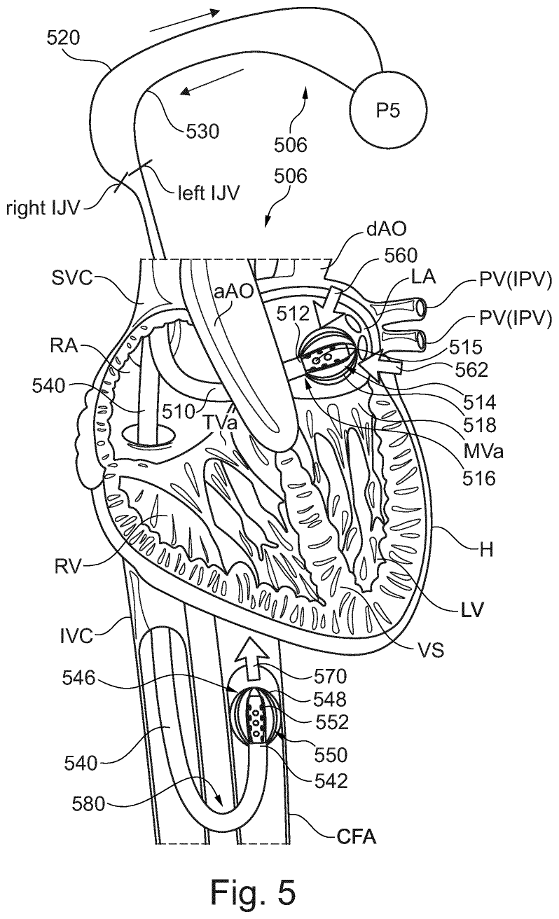

FIG. 5 a transcaval extra corporeal blood flow circuitry comprising two single lumen cannulas both inserted through jugular veins,

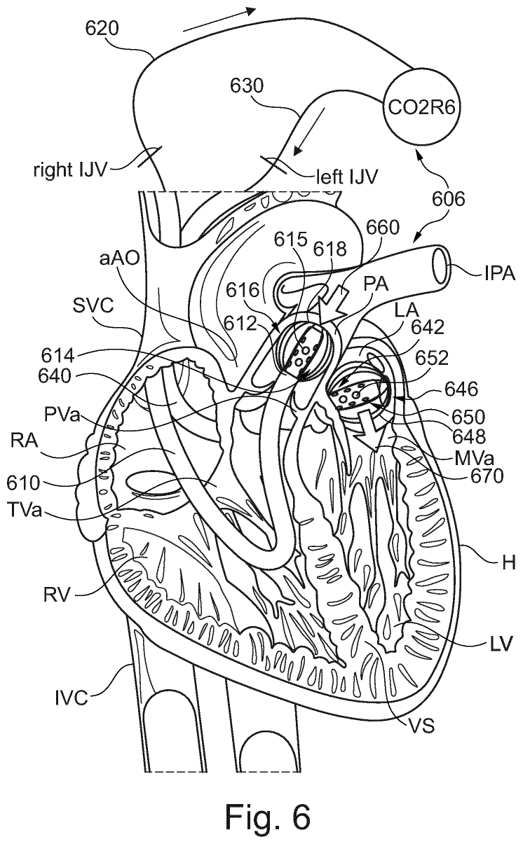

FIG. 6 an extra corporeal lung assist blood flow circuitry without pump comprising two single lumen cannulas and a carbon dioxide removal device,

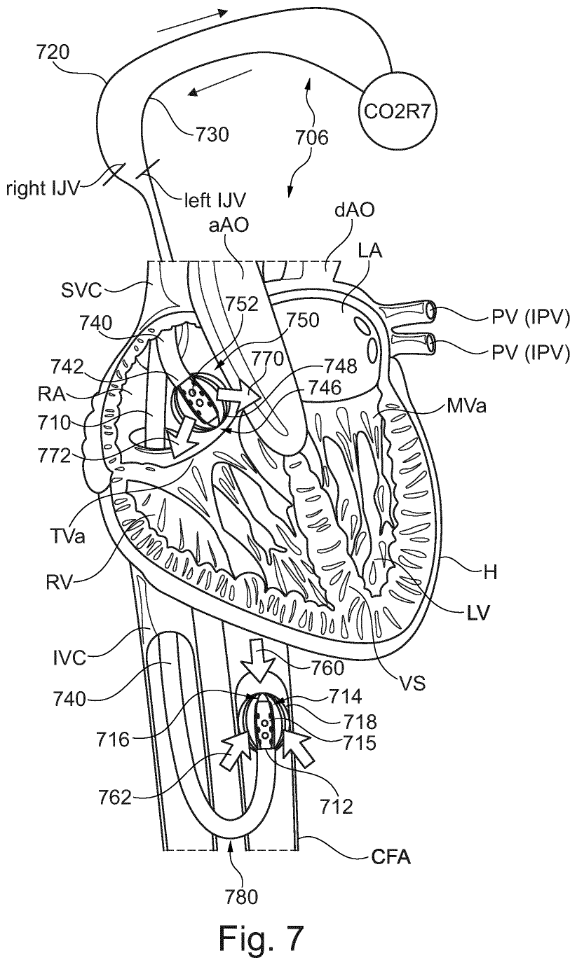

FIG. 7 a transcaval extra corporeal lung assist blood flow circuitry without pump comprising two single lumen cannulas and a carbon dioxide removal device,

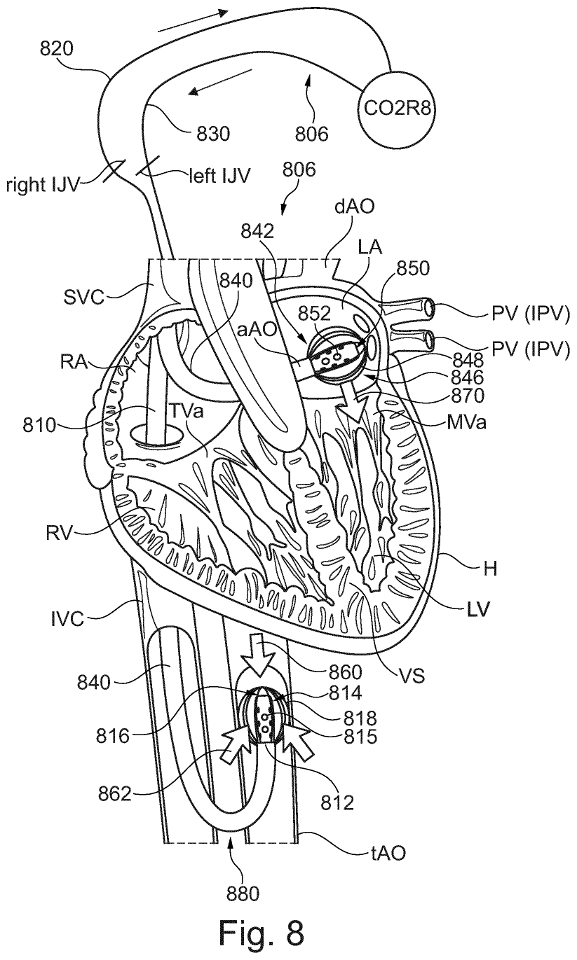

FIG. 8 a transcaval transseptal extra corporeal lung assist blood flow circuitry without pump comprising two single lumen cannulas and a carbon dioxide removal device,

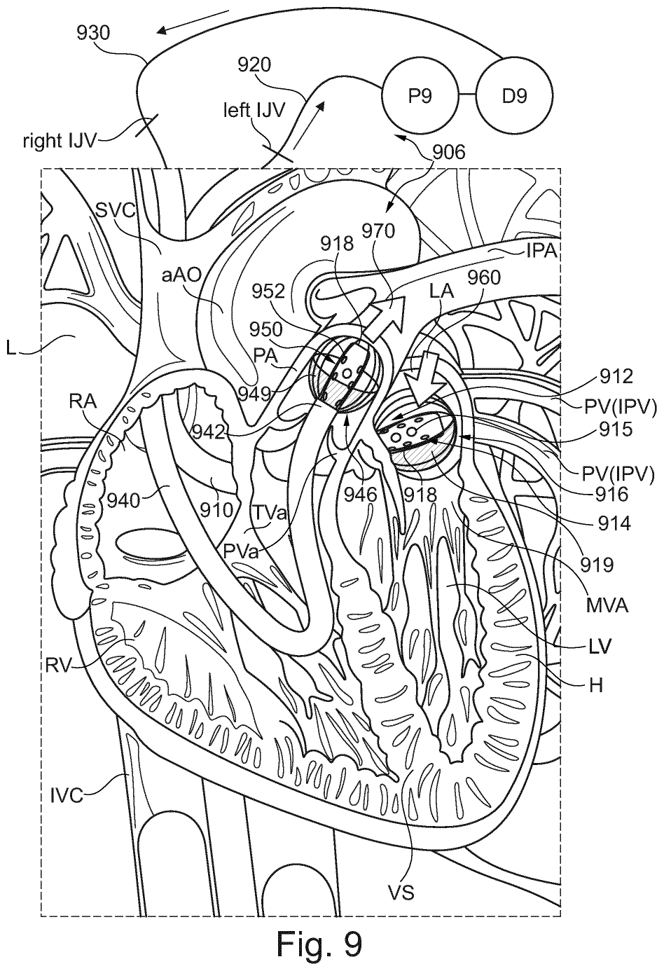

FIG. 9 an extra corporeal circular lung perfusion blood flow circuitry comprising two single lumen cannulas, a pump and a further device,

FIG. 10 an extra corporeal retrograde lung perfusion circular blood flow circuitry comprising two dual lumen cannulas, an alternative embodiment with antegrade lung perfusion, an alternative embodiment with lobe dedicated lung perfusion and an embodiment for right ventricle assist,

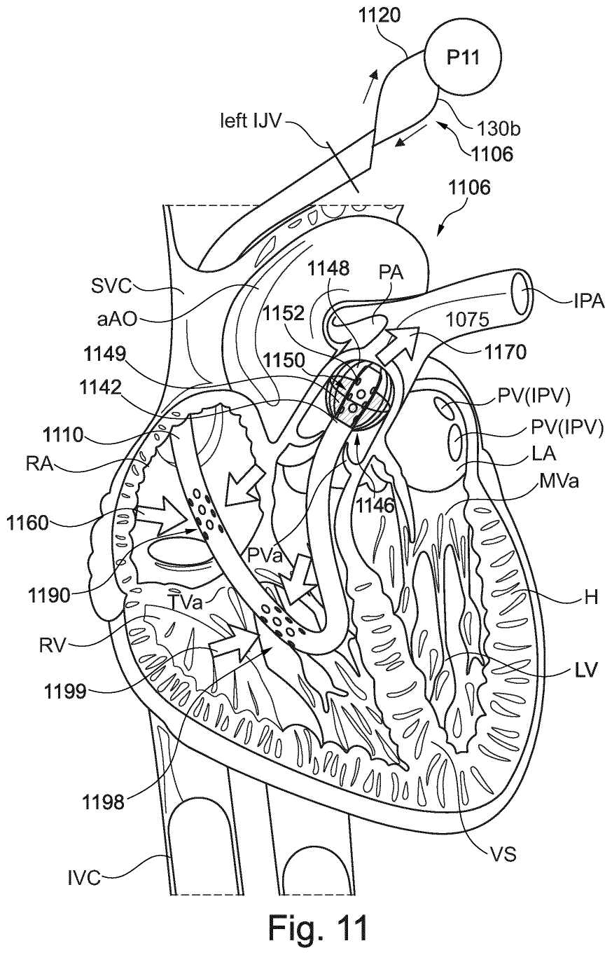

FIG. 11 a right ventricle assist circuitry with one inlet stage or with multi inlet stages,

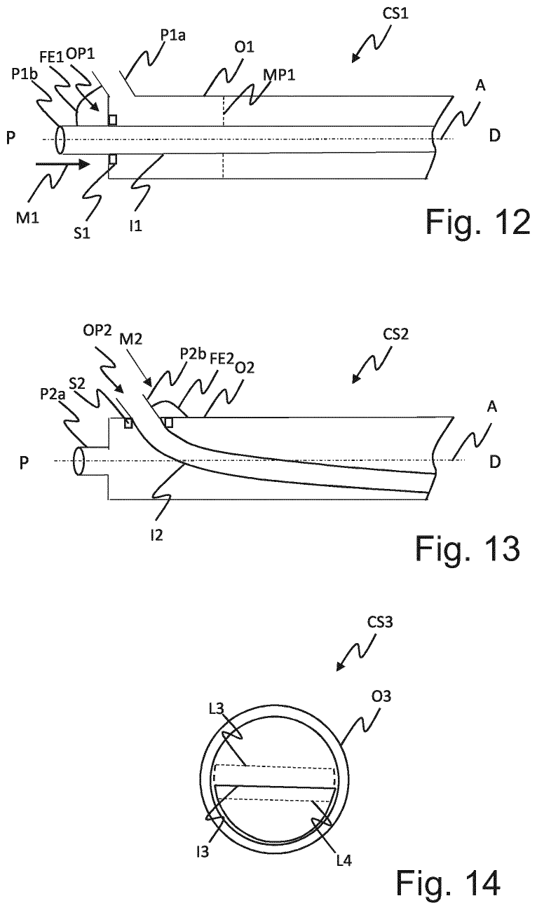

FIG. 12 a cannula system having cannulas that are arranged coaxially,

FIG. 13 a cannula system having an inner (second) cannula that is arranged loosely within an outer (first) cannula,

FIG. 14 a cross section of another cannula system,

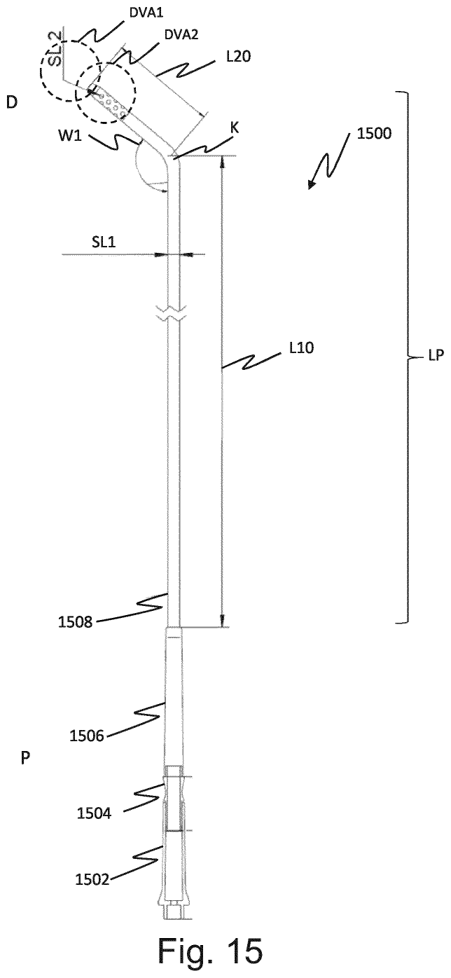

FIG. 15 an embodiment of a dual lumen system comprising at least one pre bended cannula,

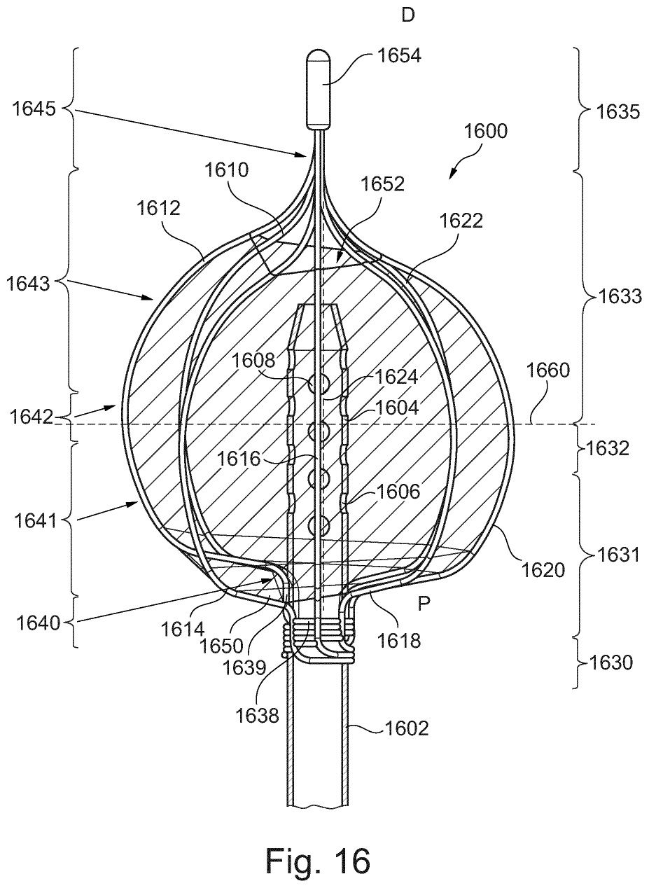

FIG. 16 a cage arrangement comprising a membrane having an opening that faces distally,

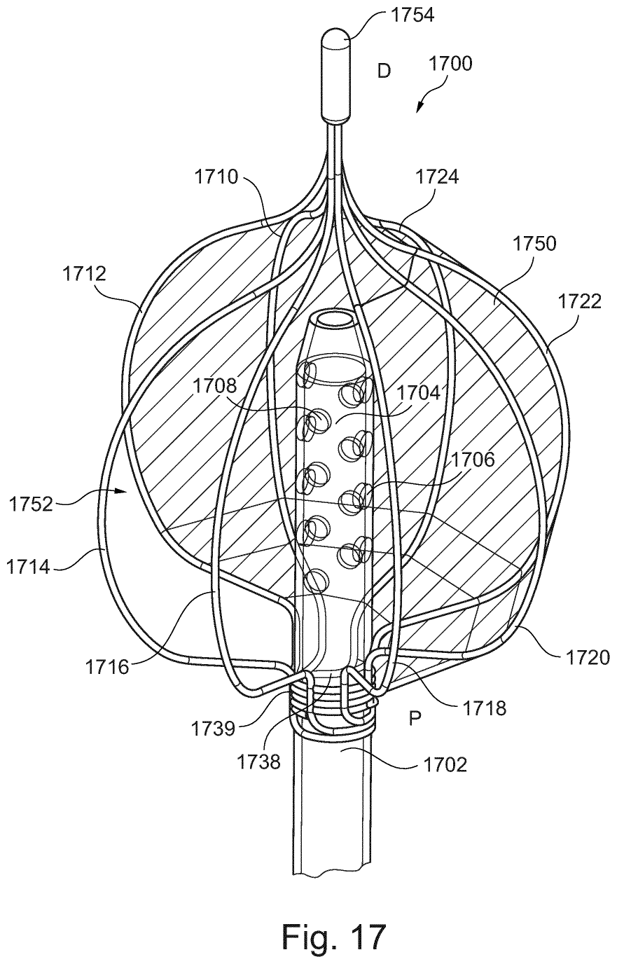

FIG. 17 a cage arrangement comprising a membrane having an opening that faces laterally,

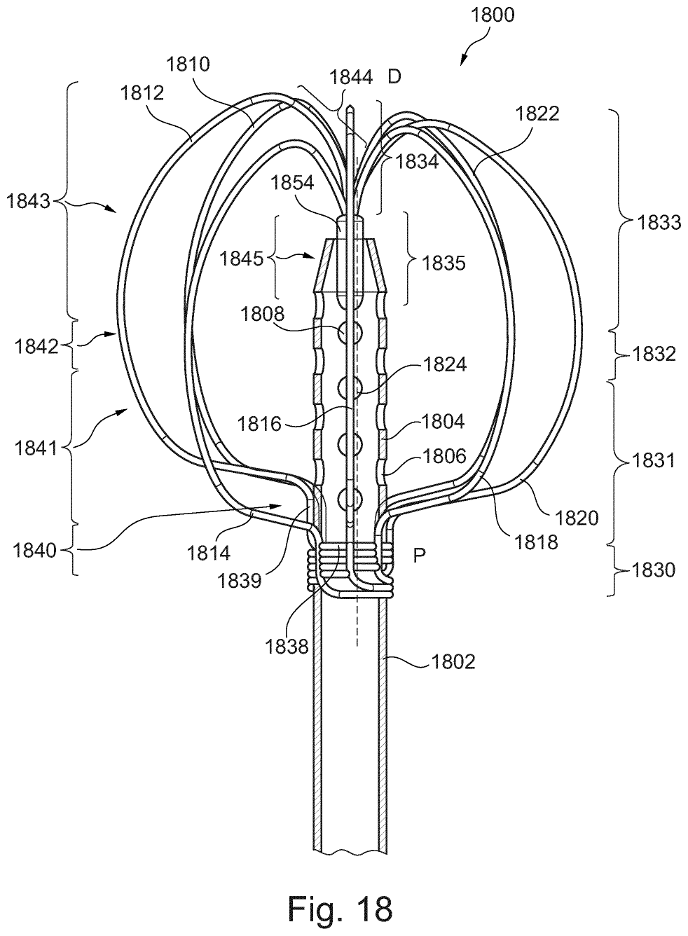

FIG. 18 a cage arrangement comprising a portion that is bended backwards,

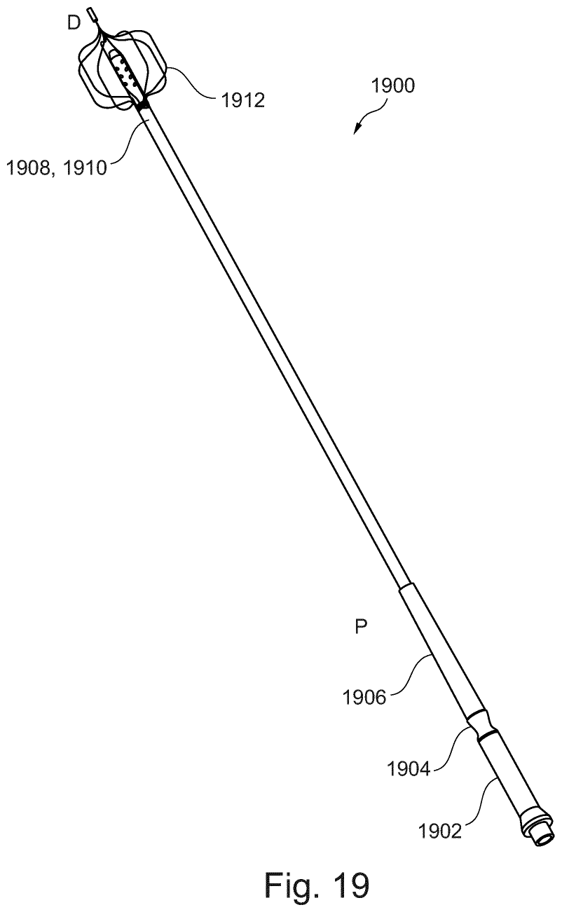

FIG. 19 a cannula comprising a cage arrangement having wires that are arranged in parallel with regard to each other,

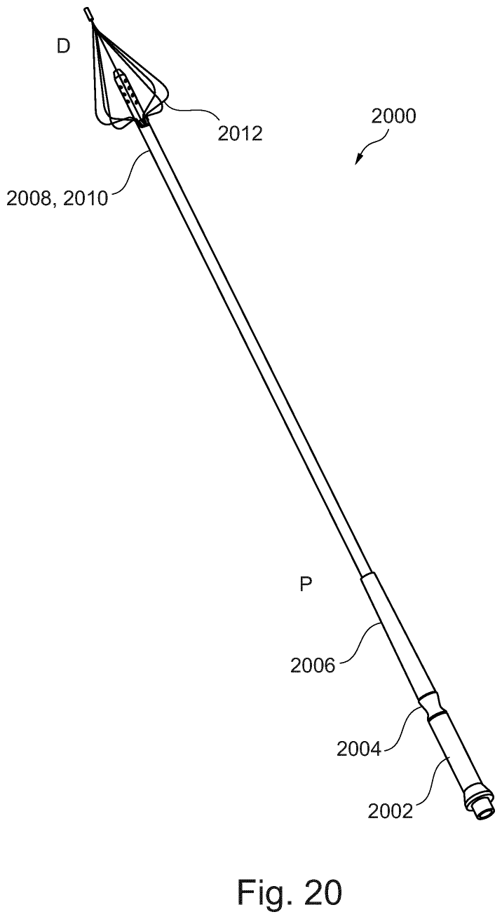

FIG. 20 a cannula comprising a cage arrangement having a cone like shape,

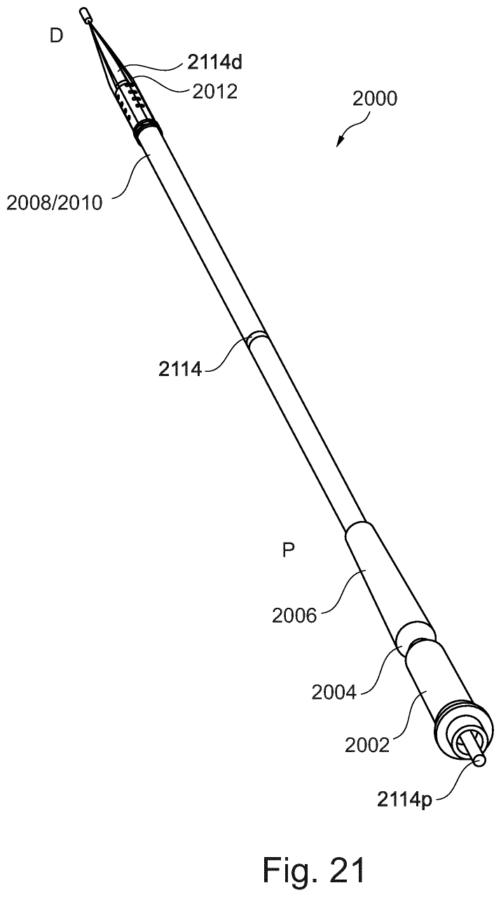

FIG. 21 the cannula of FIG. 20 in a state in which an introducer stretches the cage arrangement for introducing the cannula into a body,

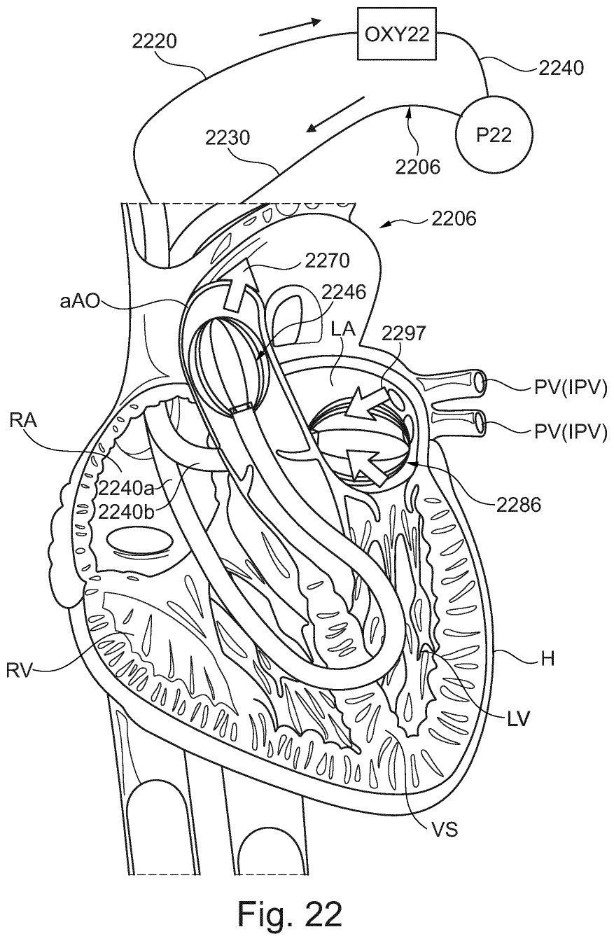

FIG. 22 an alternative embodiment wherein a cannula is pierced or punctured through a ventricle septum,

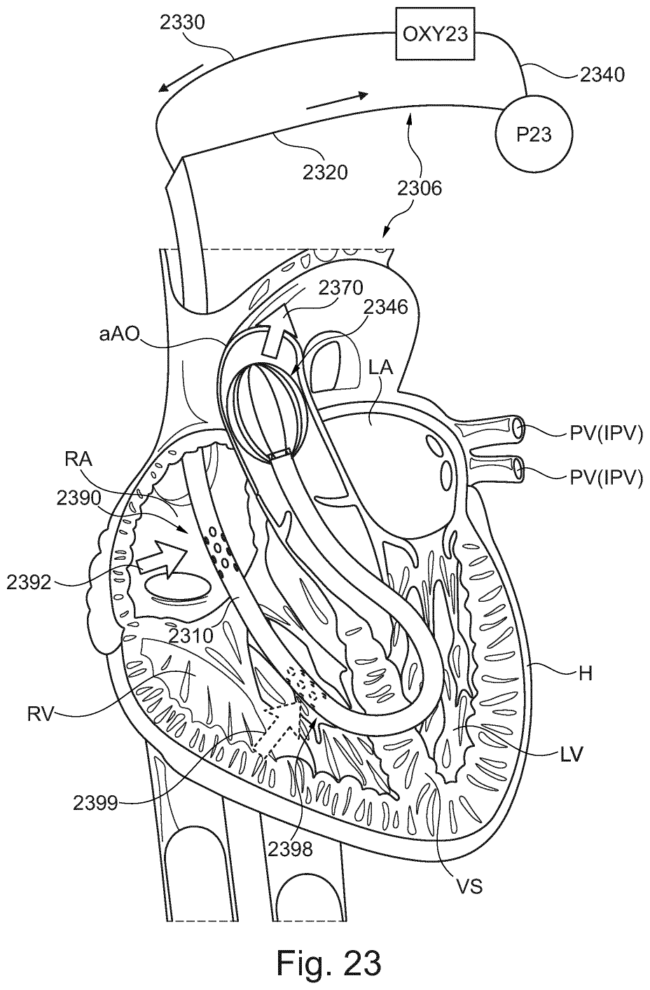

FIG. 23 a further alternative embodiment wherein a cannula is pierced or punctured through a ventricle septum,

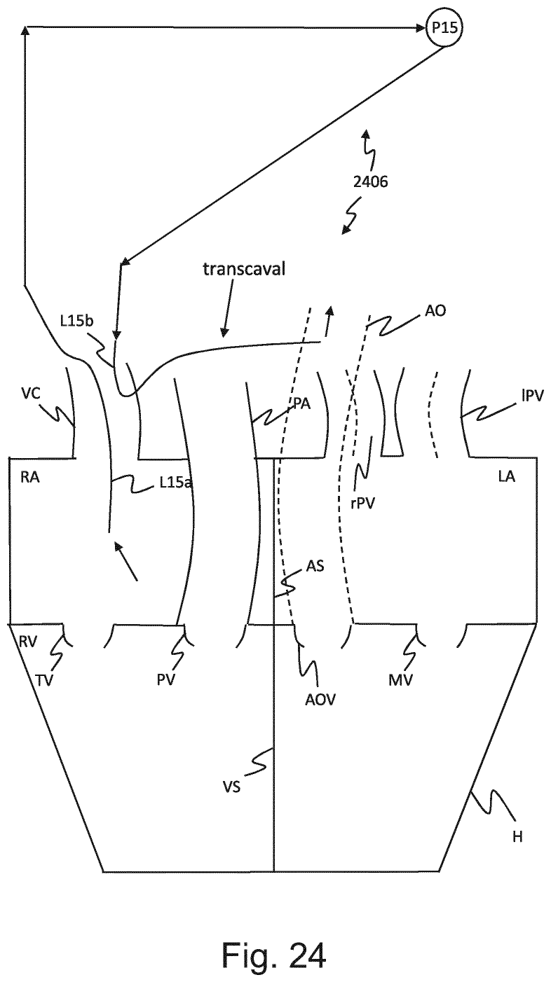

FIG. 24 an alternative embodiment wherein a cannula is punctured transcaval from vena cava to the aorta,

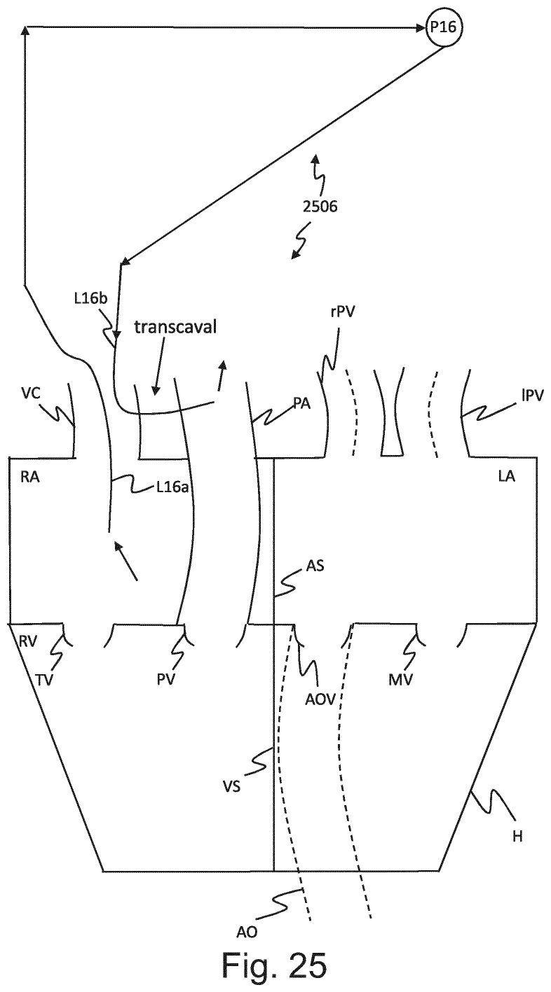

FIG. 25 a further alternative embodiment wherein a cannula is punctured transcaval from vena cava to the pulmonary artery,

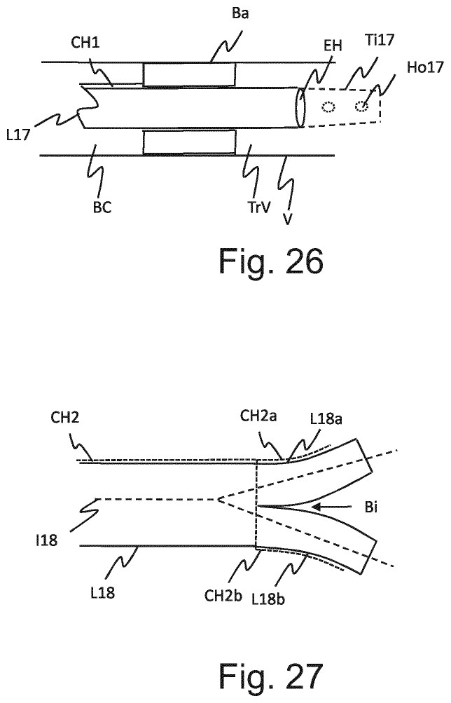

FIG. 26 a cannula that carries an inflatable expandable arrangement,

FIG. 27 a split tip cannula that carries two expandable arrangements,

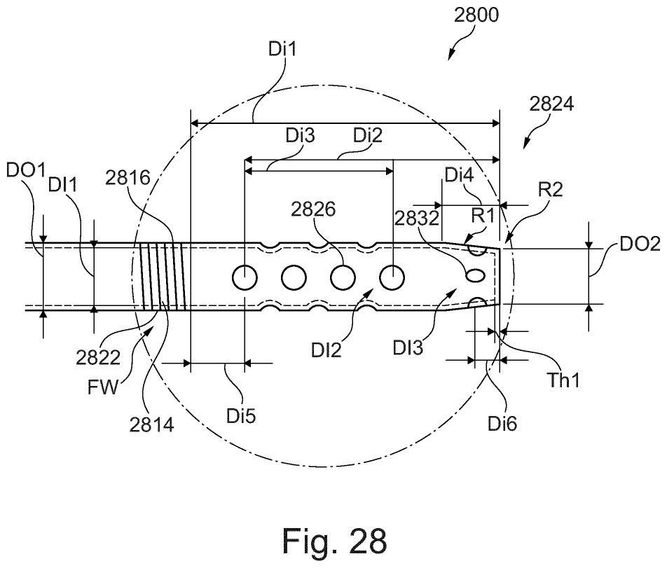

FIG. 28 a reference cannula tip,

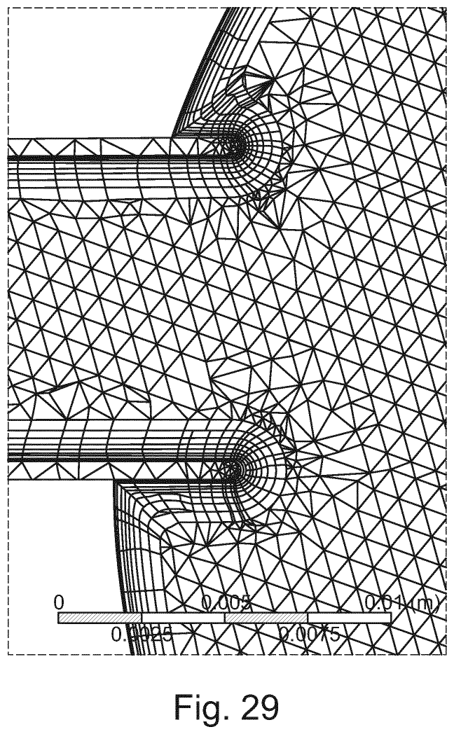

FIG. 29 a simulation mesh,

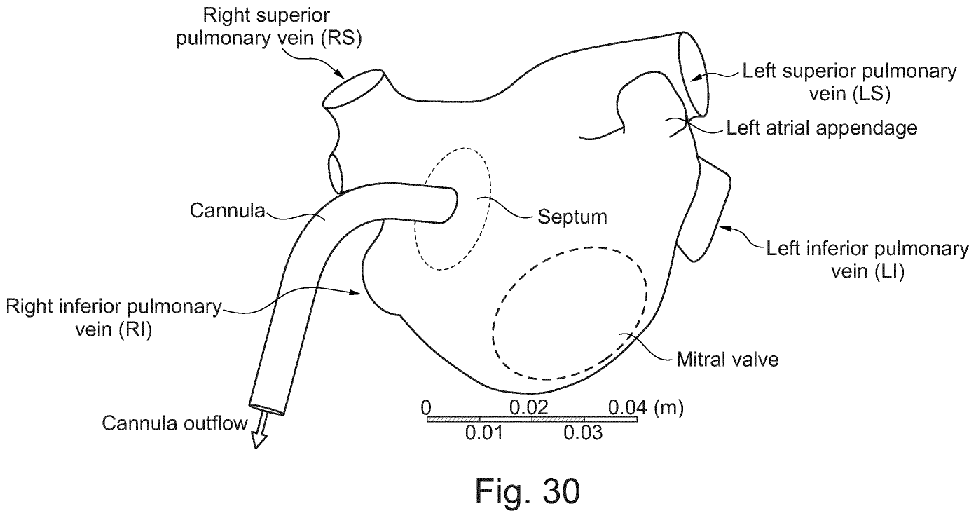

FIG. 30 the simulated left atrium of a human heart,

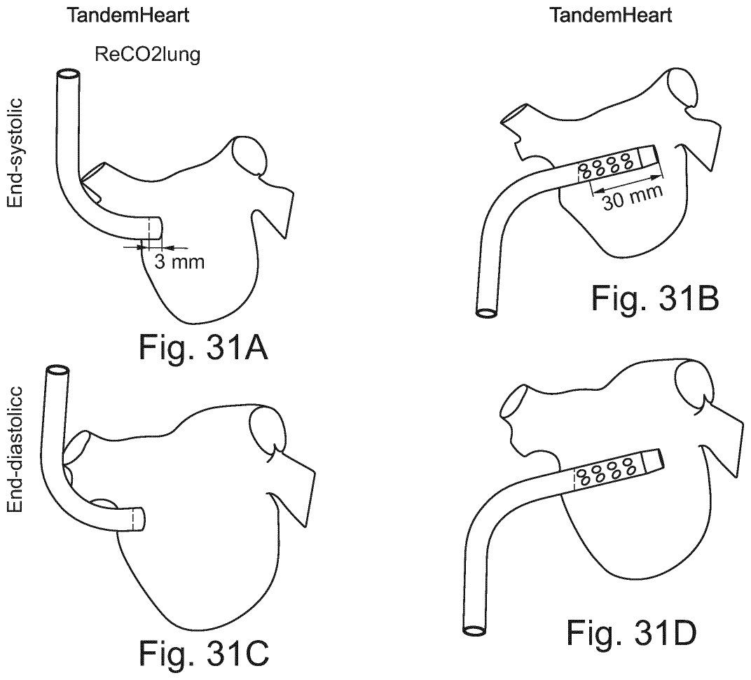

FIGS. 31A to 31D side views of cannula positions,

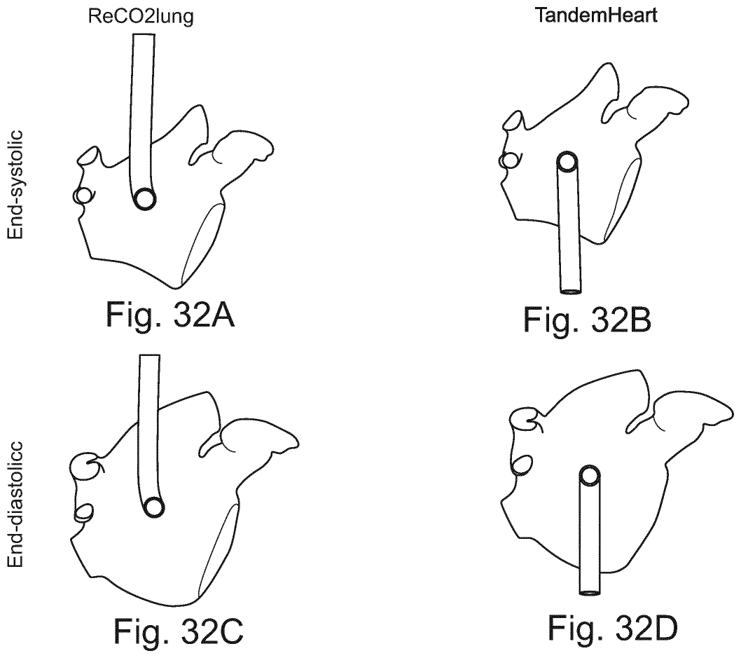

FIGS. 32A to 32D lateral views of cannula positions,

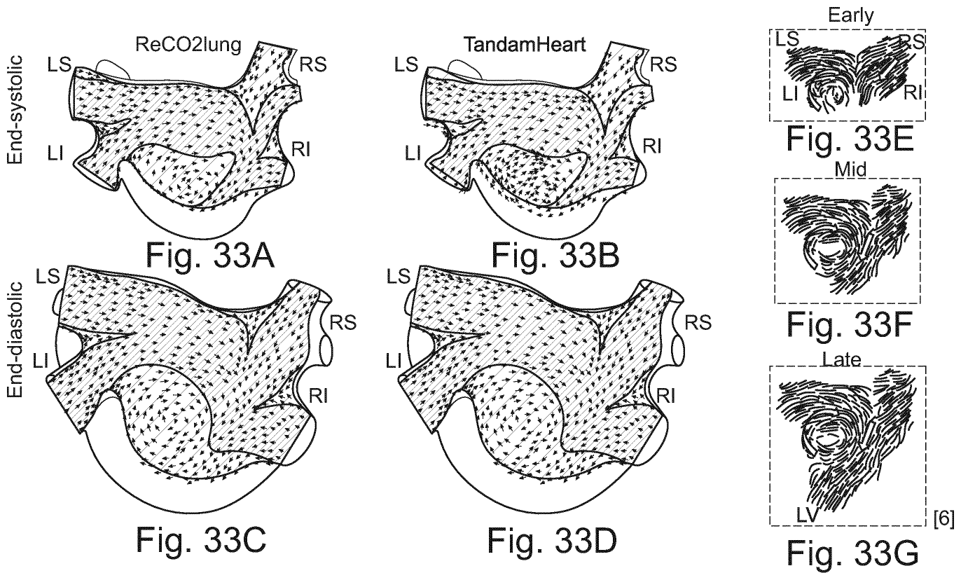

FIGS. 33A to 33G flow patterns within the left atrium showing vortexes,

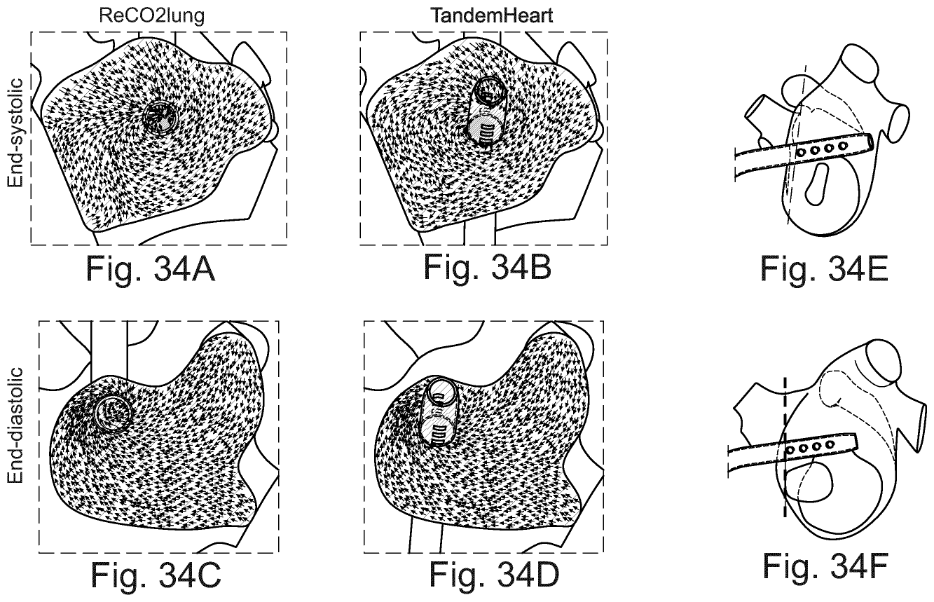

FIGS. 34A to 34F flow patterns around cannulas within the left atrium,

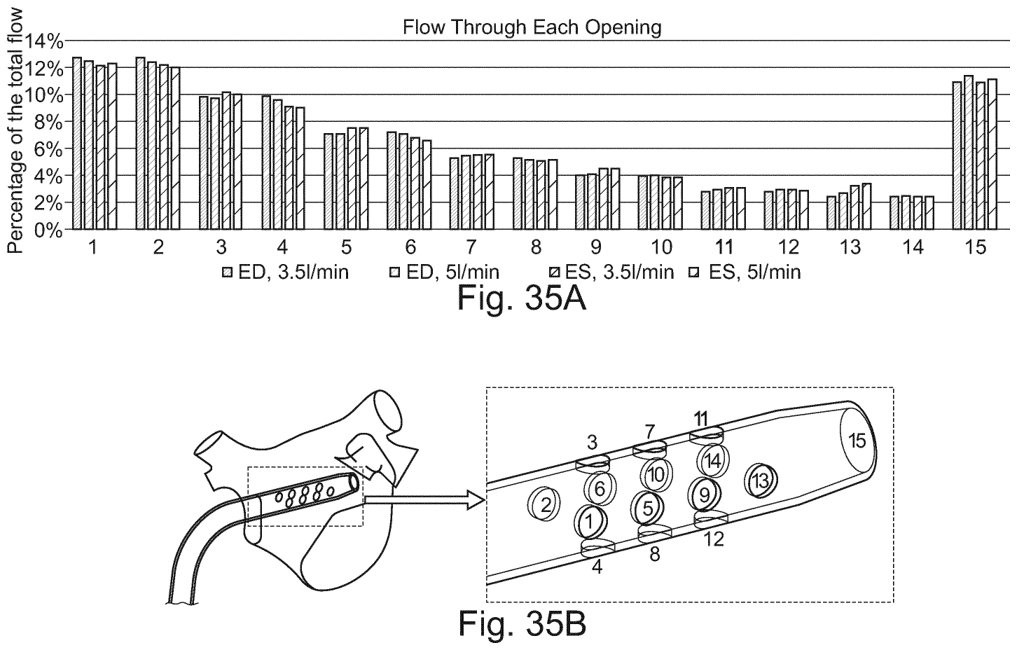

FIGS. 35A and 35B flow through each hole of a cannula tip having side-hole and one end hole as well as the numbering of the holes of the cannula tip,

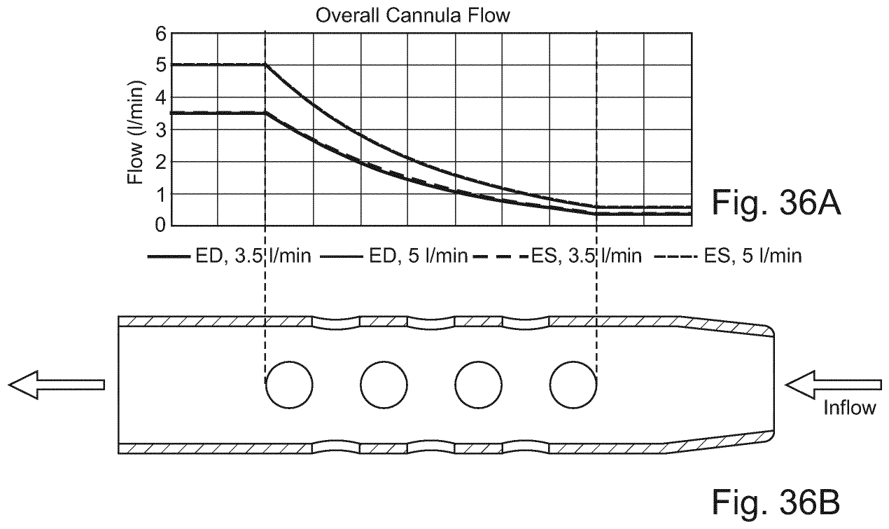

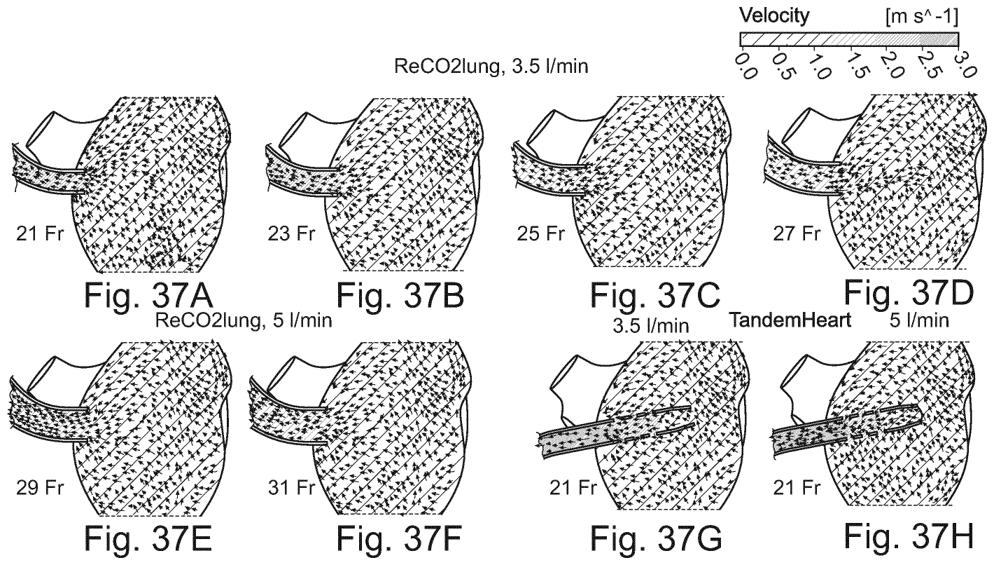

FIGS. 36A and 36B the flow into a TandemHeart® cannula,

FIGS. 37A to 37H end-systolic steady state flow patterns,

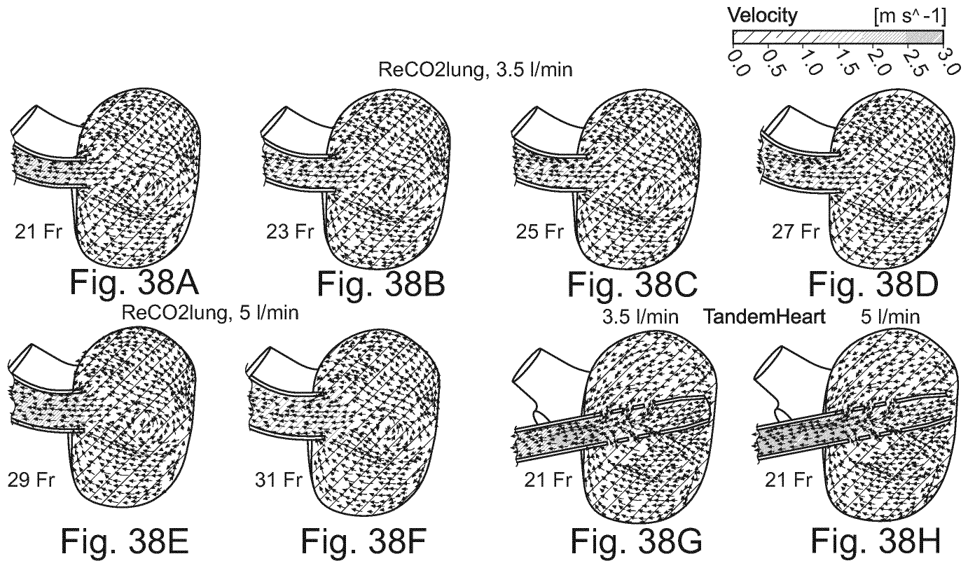

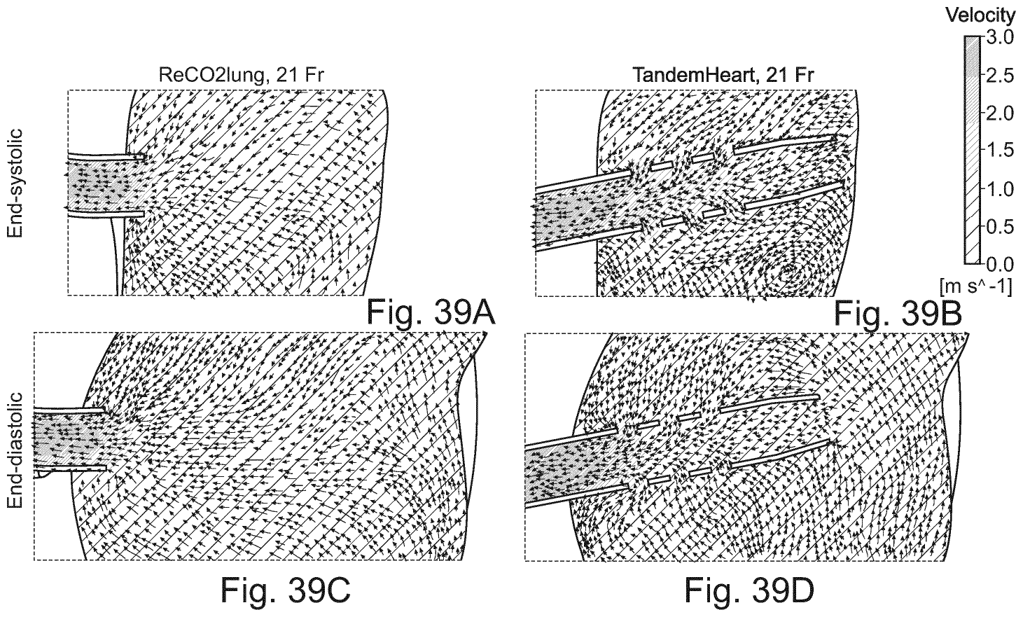

FIGS. 38A to 38H end-diastolic steady state flow patterns,

FIGS. 39A to 39D flows at the cannula tip for an overall flow of 3.5 l per minute,

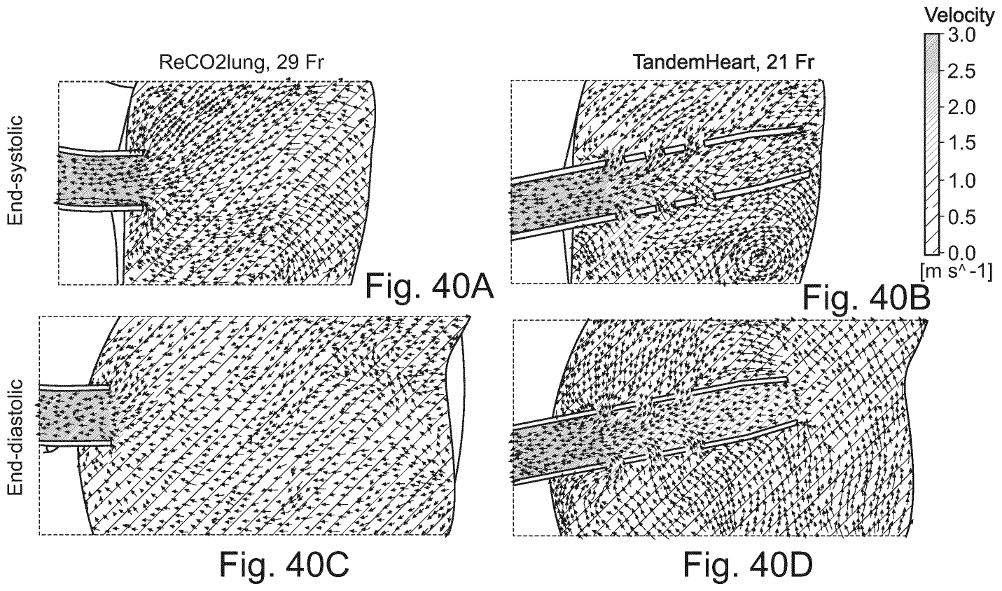

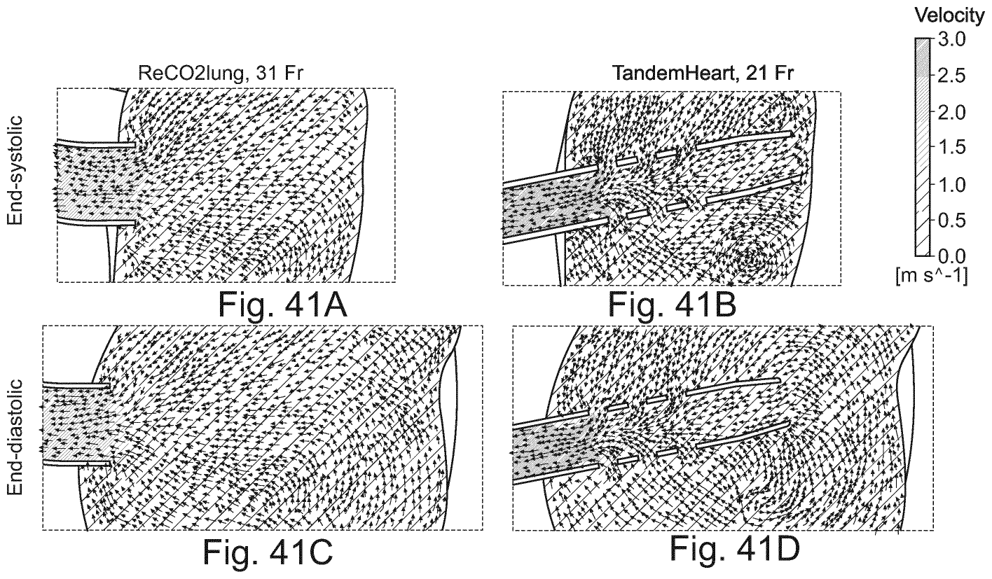

FIGS. 40A to 40D flows at the cannula tip for an overall flow of 5l per minute, ReCO2lung cannula 29 Fr,

FIGS. 41A to 41D flows at the cannula tip for an overall flow of 5l per minute, ReCO₂Lung® cannula 31 Fr,

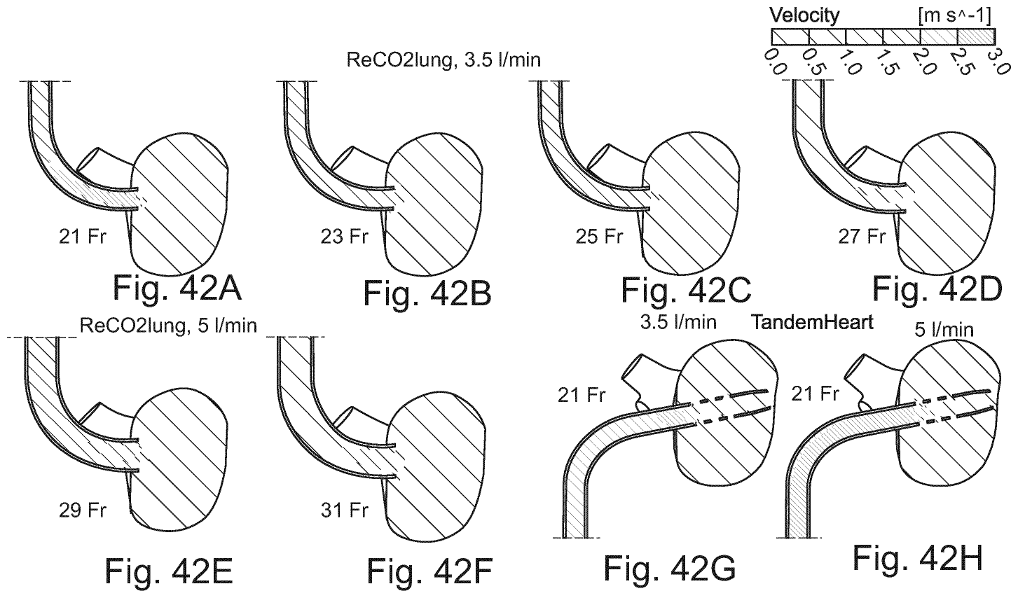

FIGS. 42A to 42H end-systolic cannula flows with steady-state velocity,

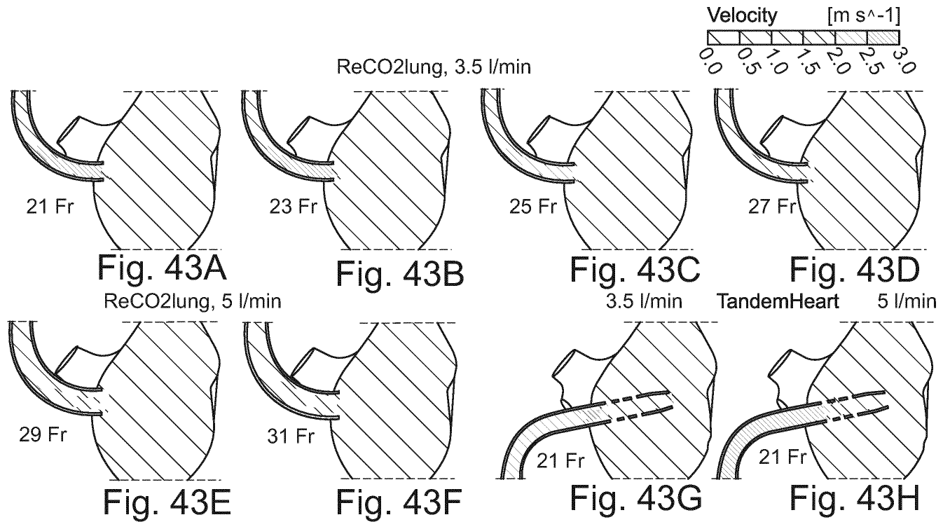

FIGS. 43A to 43H end-diastolic cannula flows with steady-state velocity,

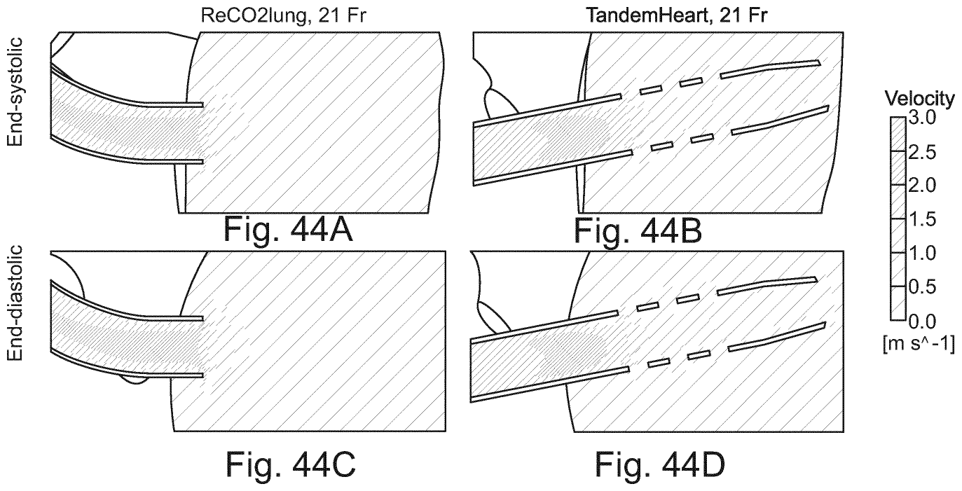

FIGS. 44A to 44D comparison cannula flow 3.5 liter per minute ReCO₂Lung® cannula 21 Fr,

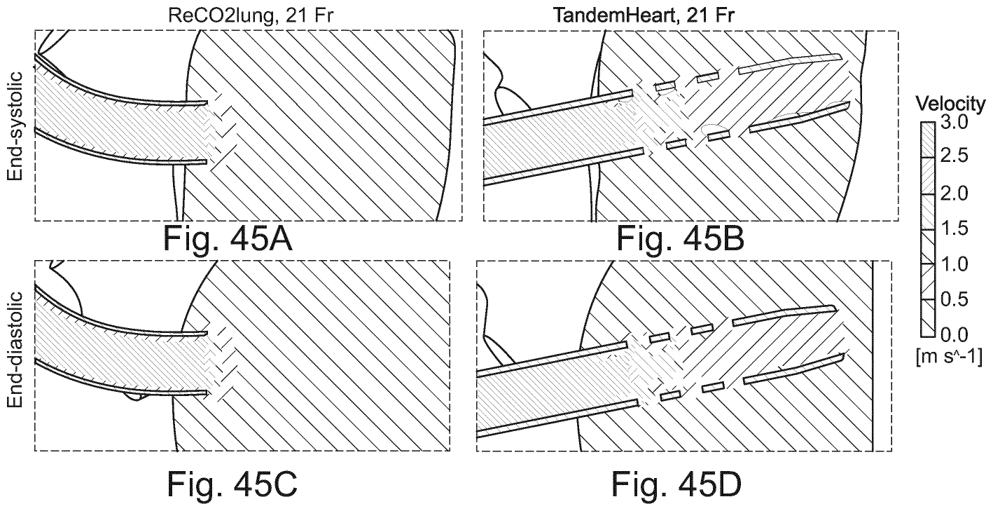

FIGS. 45A to 45D comparison cannula flow 5 liter per minute, ReCO₂Lung® cannula 29 Fr,

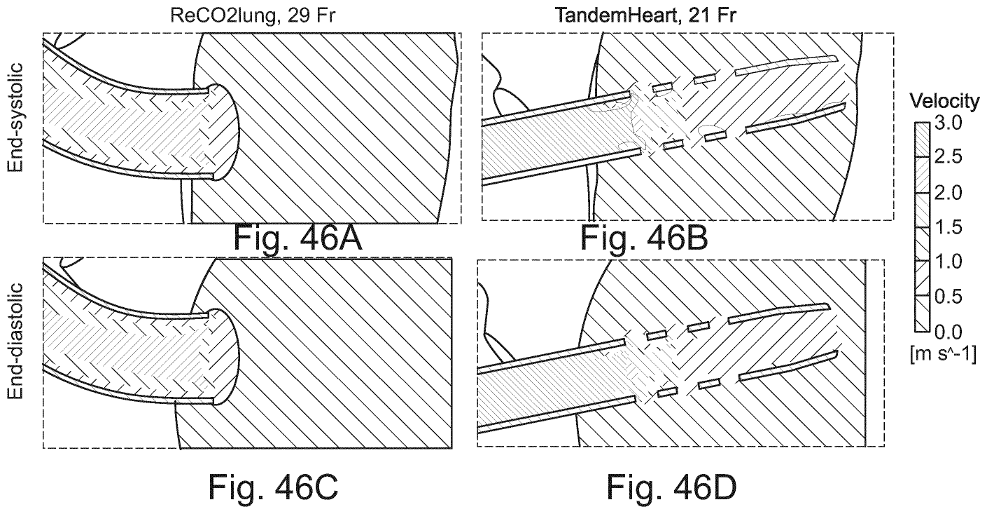

FIGS. 46A to 46D comparison cannula flow 5 liter per minute, ReCO₂Lung® cannula 31 Fr,

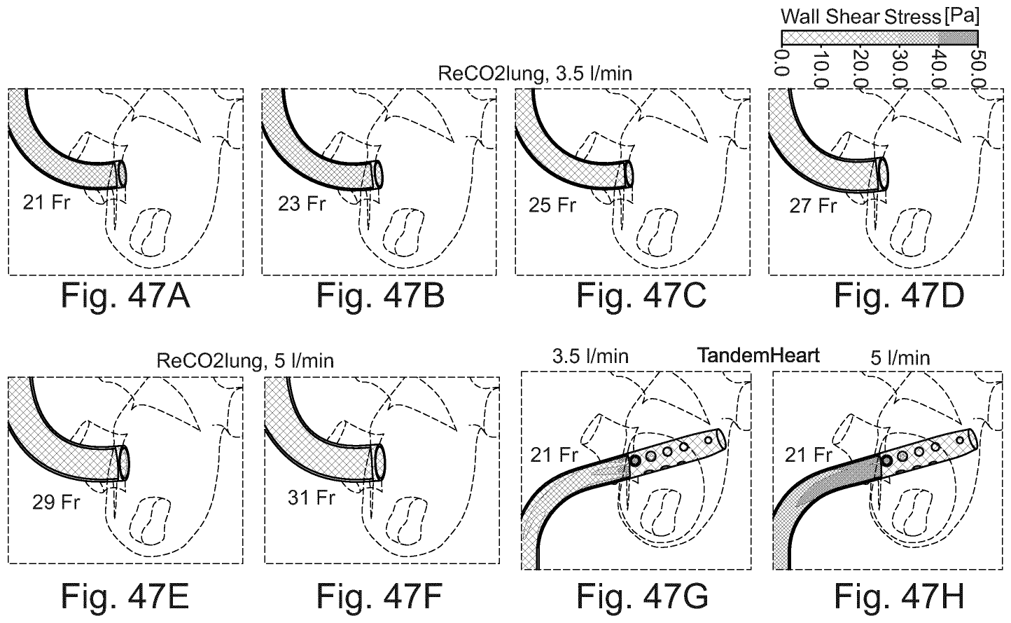

FIGS. 47A to 47H steady-state wall shear stress, end-systolic,

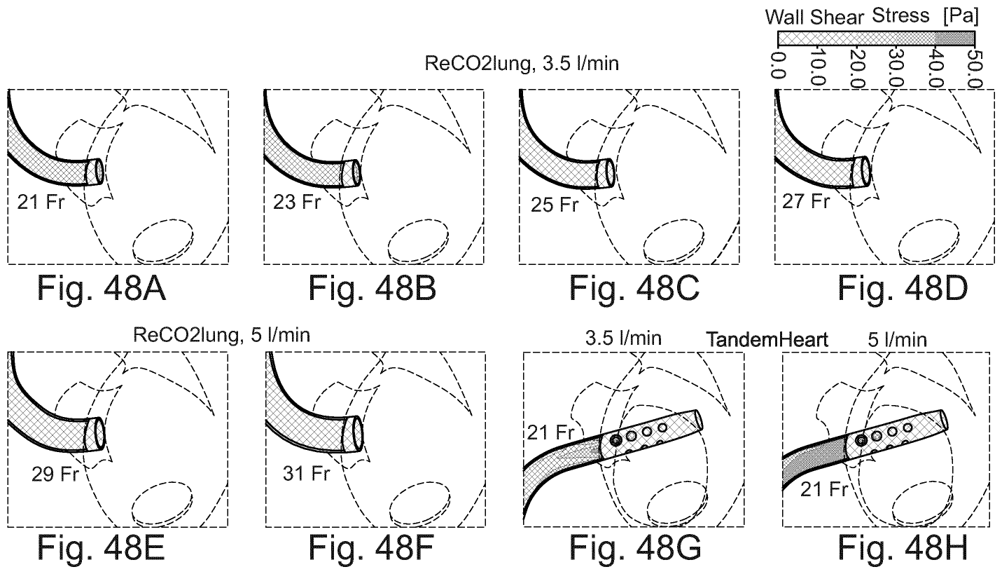

FIGS. 48A to 48H steady-state wall shear stress, end-diastolic,

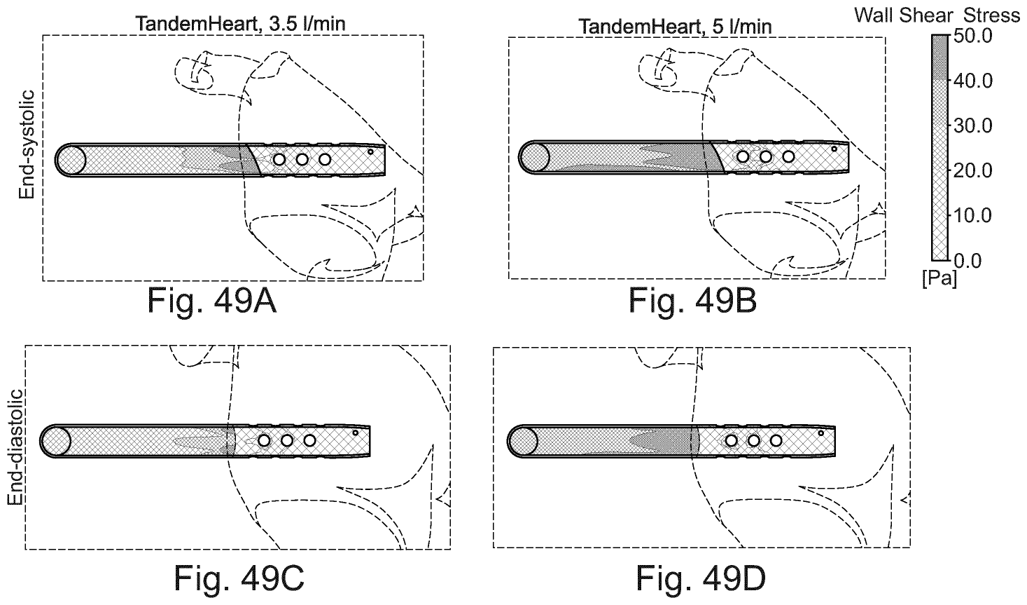

FIGS. 49A to 49D wall shear stress TandemHeart® cannula,

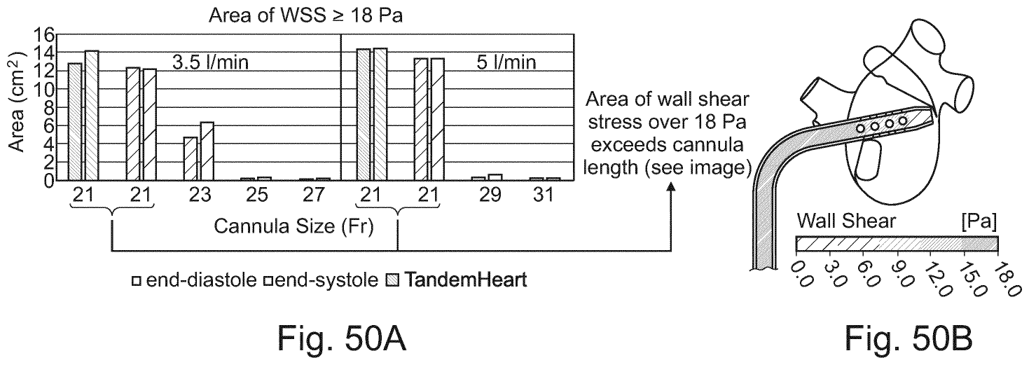

FIGS. 50A and 50B steady-state wall shear stress equal to or above 18 Pa,

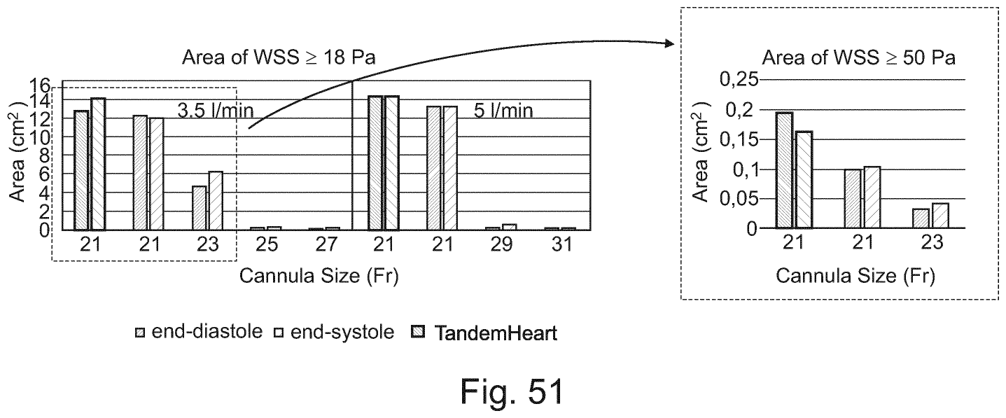

FIG. 51 steady-state wall shear stress equal to or above 18 Pa and equal to or above 50 Pa,

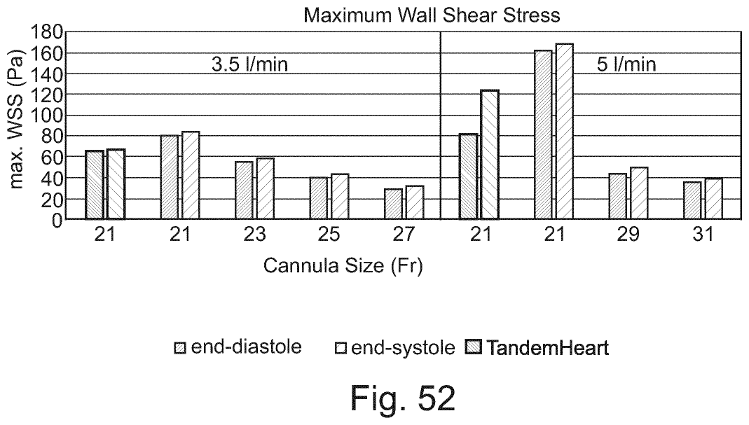

FIG. 52 maximum wall shear stress,

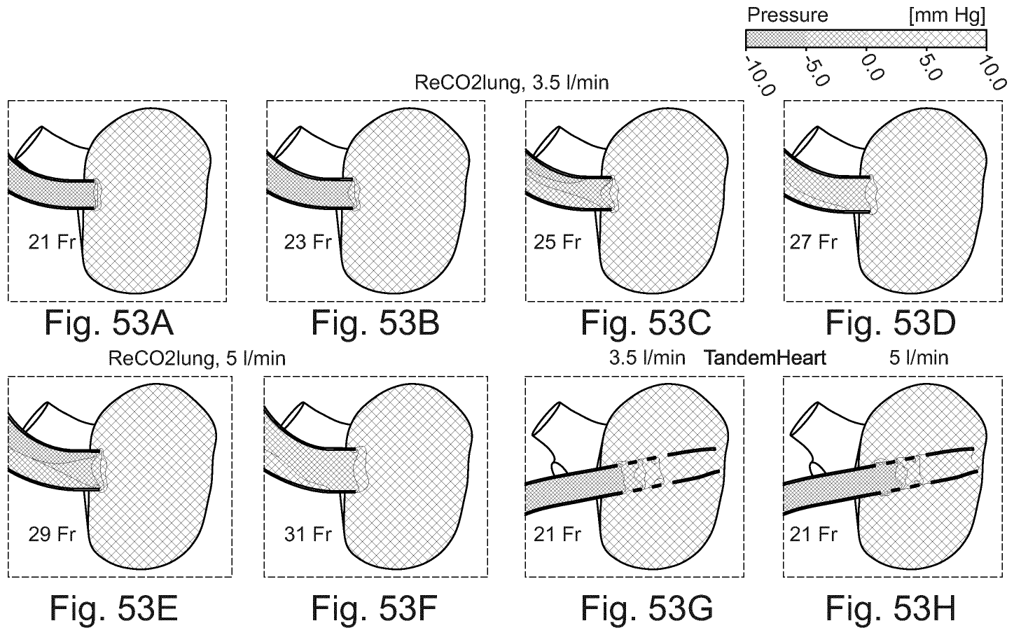

FIGS. 53A to 53H end-systolic stead-state pressure distribution,

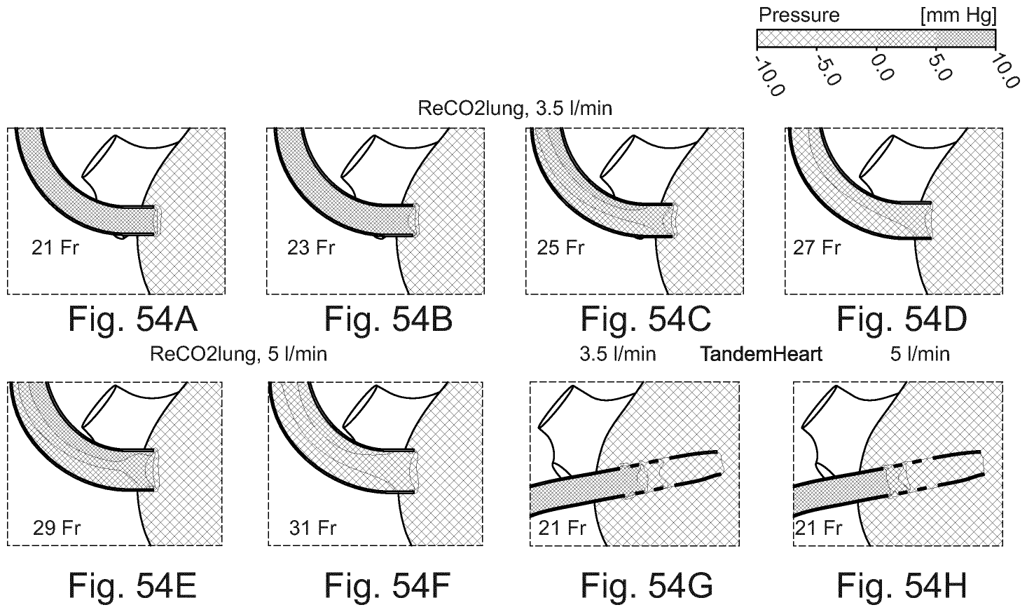

FIGS. 54A to 54H end-diastolic stead-state pressure distribution,

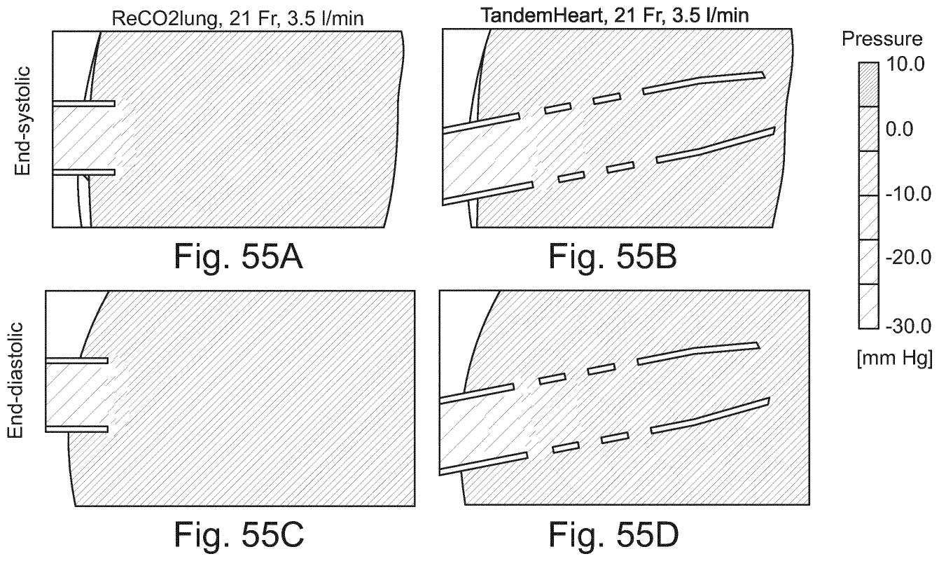

FIGS. 55A to 55D steady-state pressure distribution at the cannula tip,

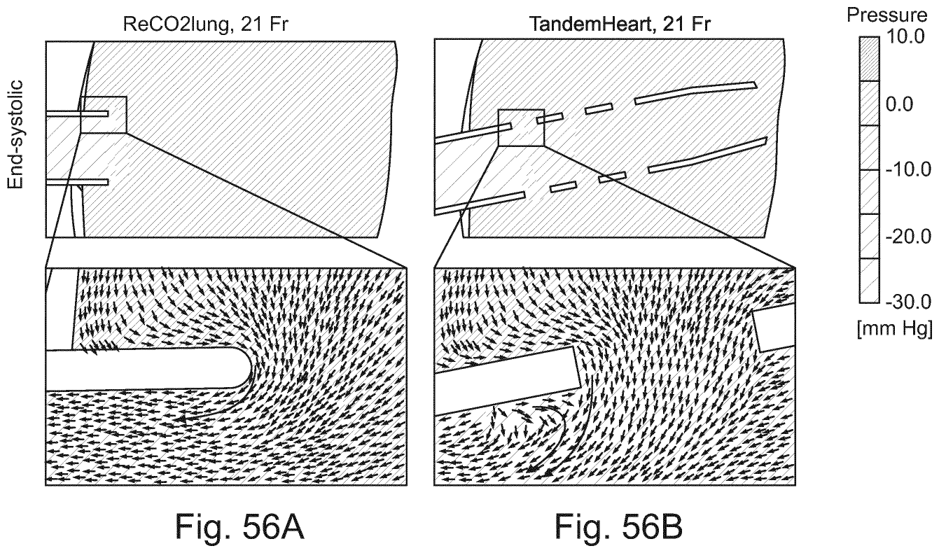

FIGS. 56A and 56B steady-state pressure distribution at the cannula tip, magnification,

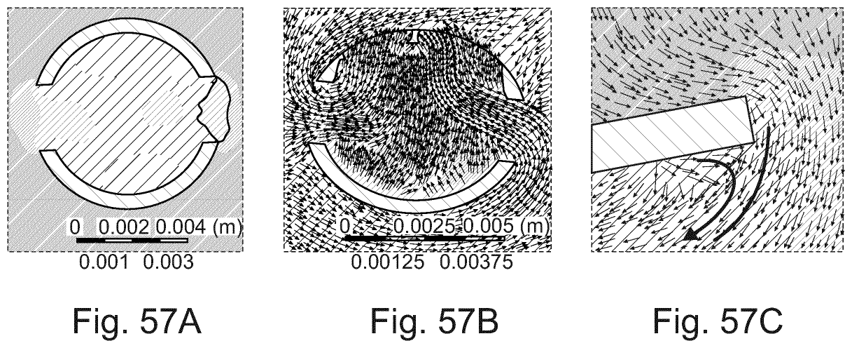

FIGS. 57A to 57D steady-state pressure distribution at the cannula tip flow pattern TandemHeart® cannula,

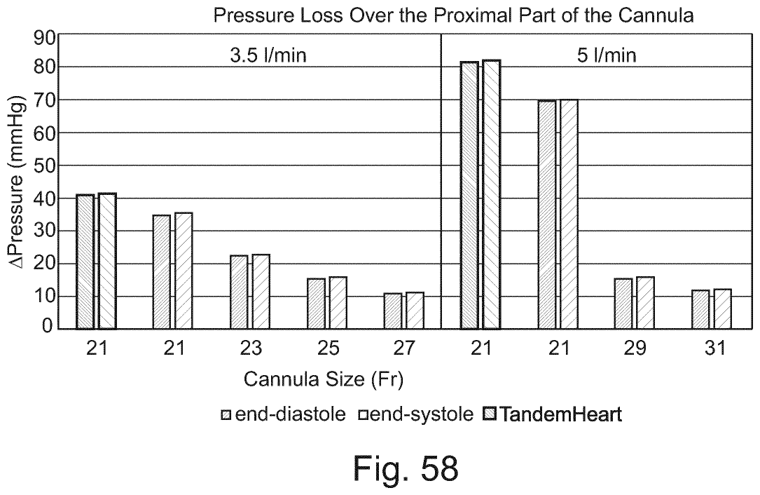

FIG. 58 pressure loss,

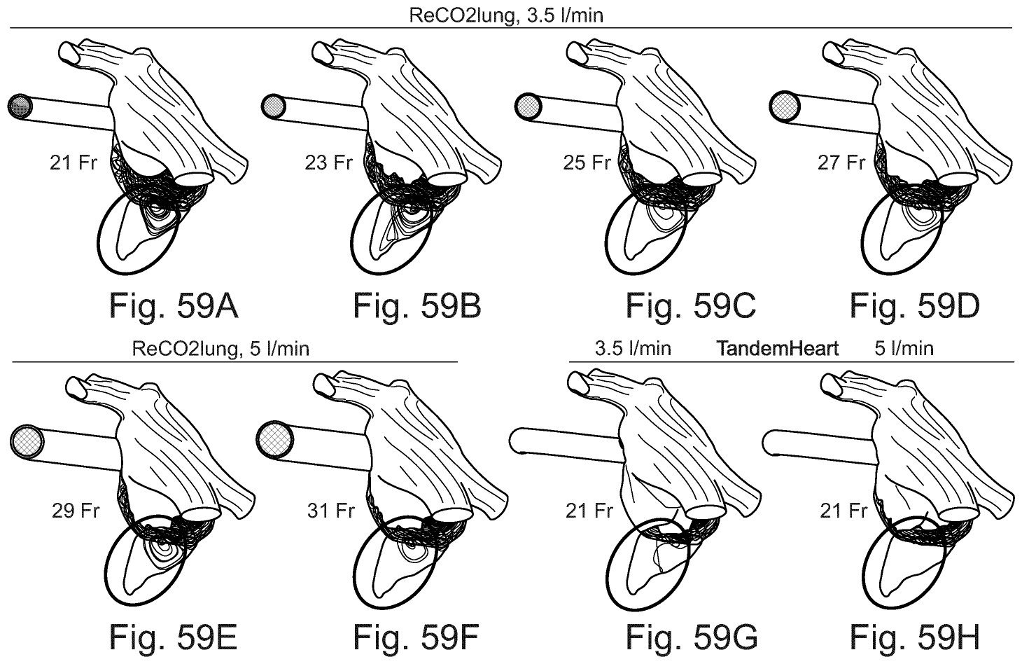

FIGS. 59A to 59H end-systolic left atrial appendage steady-state flow fields,

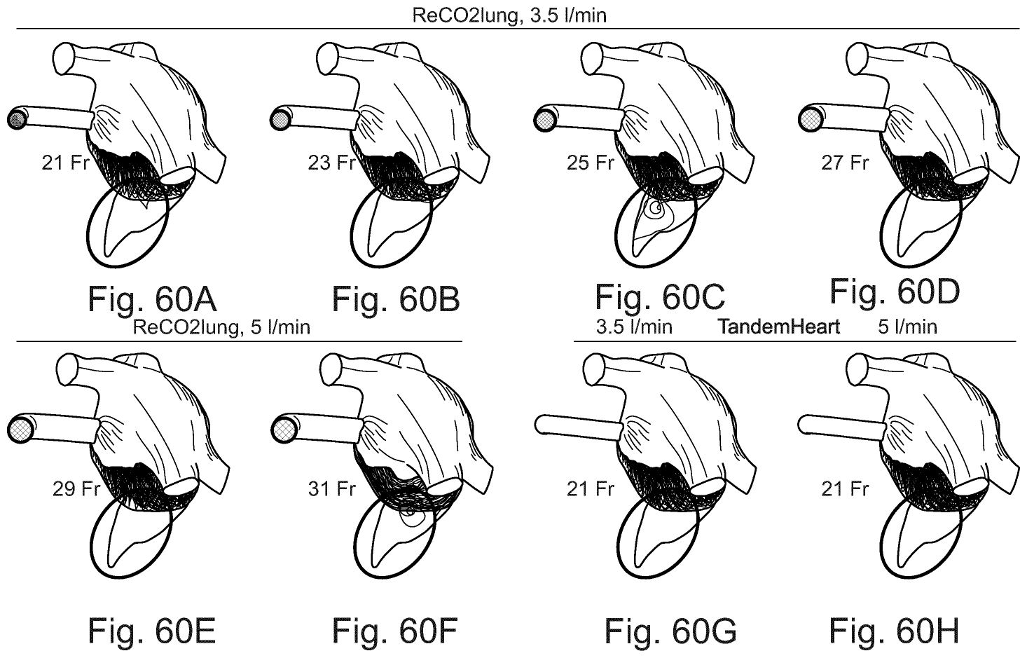

FIGS. 60A to 60H end-diastolic left atrial appendage steady-state flow fields,

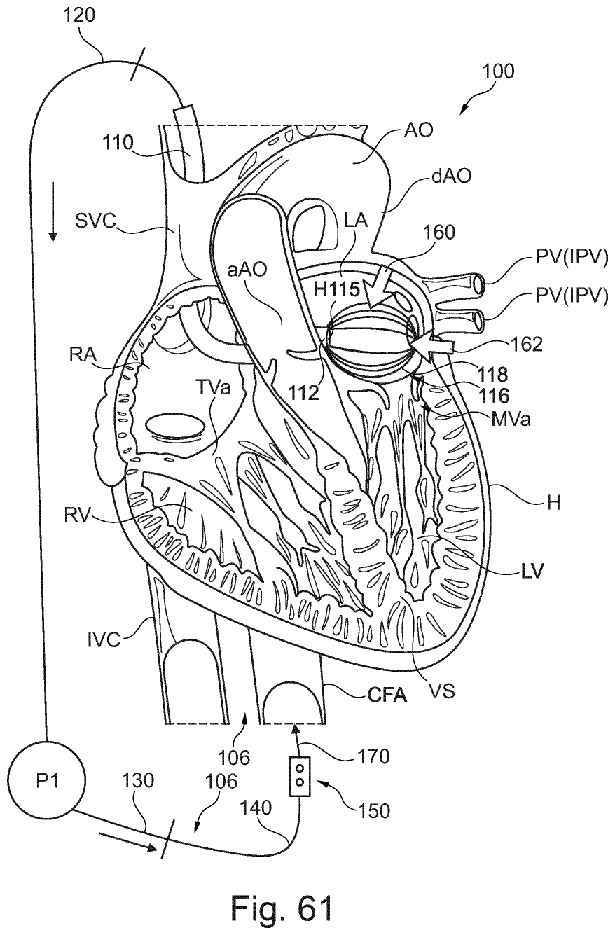

FIGS. 61 to 71 see FIGS. 1 to 11 but without a tip with side holes within the cage arrangements, and

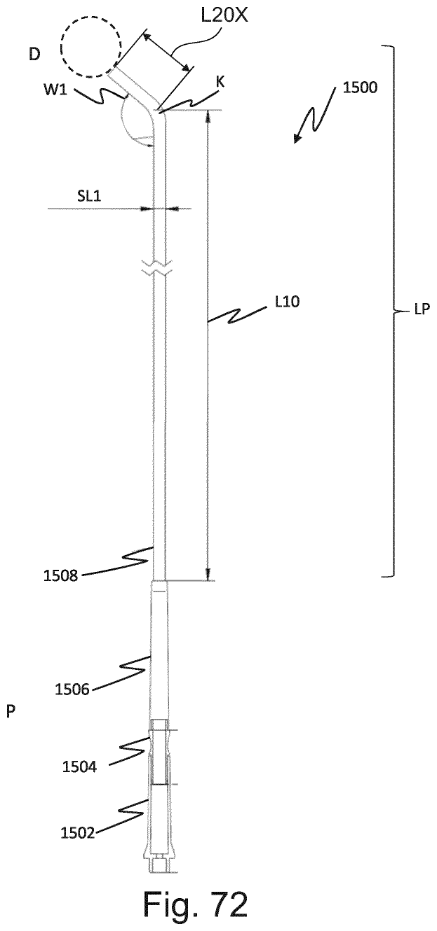

FIGS. 72 to 78 see FIGS. 15 to 21 but without tip with side holes within cage arrangements.

### A) LEFT AND BI VENTRICLE ASSIST

FIG. 1 illustrates an extra corporeal circular blood flow circuitry 106 comprising a single lumen cannula 110 carrying a cage arrangement 116 near at least one inlet port, a blood pump P1 and a second single lumen cannula 140 that has at least one outlet port in the artery of the lower trunk. First single lumen cannula 110 is inserted through right internal jugular vein IJV, superior vena cava SVC, right atrium RA, transseptal, i.e. through the septum between right atrium RA and left atrium LA, into left atrium LA. A guide wire (not shown) may be used to guide cannula 110 to its final position. Alternatively, cannula 110 may be inserted through right subclavian vein. Blood is withdrawn by suction from left atrium LA through cannula 110, see arrows 160, 162.

Cannula 140 is inserted through the right femoral artery into common femoral artery CFA where blood is injected in a retrograde fashion into common femoral artery CFA, see arrows 170 and 172.

A body 100 comprises a head 102 and a trunk 104, see FIG. 4. The heart H of a patient is located within trunk 104. The patient may be a male or female adult or a child. The heart H comprises the following chambers:
right atrium RA,
right ventricle RV,
left atrium LA, and
left ventricle LV.

The atrial septum is between right atrium RA and left atrium LA. The ventricle septum is between right ventricle RV and left ventricle LV.

The following valves of heart H are shown:
tricuspid valve TVa between right atrium RA and right ventricle RV, and
mitral valve MVa.

The aortic valve AVa between aorta AO and left ventricle LV is not shown. The same applies for pulmonary valve PVa between right ventricle RV and pulmonary artery PA that is omitted in order to not to obscure the view to the parts of heart H that are relevant in the shown embodiment. Left pulmonary vein PV is shown in FIG. 1. Blood that is enriched with oxygen comes from lung L into left atrium LA through pulmonary vein PV. This is an exception in that a vein transports blood that comprises more oxygen than blood in a comparable artery. The description of heart H will not be repeated below. However, it is clear that this description is valid for all FIGS. 1 to 11.

An optional inlet tip 114 may be mounted on distal end 112 of cannula 110. Inlet tip 114 may comprise a plurality of inlet holes 115 in its side wall. Additionally, there may be a hole within distal end 112 of inlet tip 114. The sum of the cross section areas of the holes of tip 114 may be greater than the inner cross section area of cannula 110 at its distal end 112, for instance greater than twice the area or the triple of the area. This means that blood can be removed even if one or more of the inlet holes 115 in inlet tip 114 is or are clogged.

However, in other embodiments no separate inlet tip 114 is used. Thus, there is only one inlet hole at distal end 112 of cannula 110. This single inlet hole would be surrounded by cage arrangement 116.

Cage arrangement 116 is one possible example. Other possible examples are described below with reference to FIGS. 16 to 20. Cage arrangement 116 may comprise for instance between 6 to 12 flexible wires, beams or bars. There may be for instance 8 cage wires 118 that span a sphere. The sphere prevents that the side wall of left atrium LA covers one of inlet holes 115 of tip 114. Furthermore, cage arrangement 116 fixes distal end 112 of cannula 110 to the septum. Thus, it is not possible that cannula 110 slides back into right atrium RA.

With reference again to FIG. 1, a tube 120 is connected to a proximal end of cannula 110 and to an inlet of pump P1. Pump P1 may be a peristaltic pump or centrifugal pump or another kind of pump, for instance a membrane pump or an axial pump. A tube 130 is connected to an outlet of pump P1 and to the proximal end of cannula 140. Pump P1 may be an electrically driven pump. There may be a control unit that controls the pumping performance, for instance depending on an ECG (electrocardiography) signal. Pump P1 may be operated in pulsed or continuous mode.

Tubes 120, 130 may be made of a flexible material or of a more rigid material. Circuitry 306 may further include one or more blood filter units or units for dialysis of blood.

Cannula 140 may comprise an optional outlet tip 150 that has the same structure as inlet tip 114 of cannula 110. This means that outlet tip 150 may comprise a plurality of outlet holes 152 in its side wall and/or on its distal end.

Extra care has to be taken because cannula 140 is inserted into an artery. Blood pressure is much higher in an artery compared to blood pressure in vein. Furthermore, blood flow from a vein is continuously but blood flow in artery is pulsed. Pulsed mode of the pump is not necessary because of the retrograde infusion.

However, pump P1 may be operated in a pulsed mode. Control may be performed depending on the rhythm of the heartbeat. A sensor may be used to detect the heartbeat, especially an electronic sensor. If heart H is in a diastolic state the counter pressure against infusion of blood into artery CFA may be weak.

Retrograde infusion of blood may not be as advantageous as antegrade infusion because water divides of the lymphatic system are formed and because of the forming of turbulences. The formation of thrombus may be facilitated by retrograde infusion. The arrangement shown in FIG. 1 may be used for patients without lung problems. Furthermore, circuitry 106 supports the left part of heart H of the patient. The arrangement shown in FIG. 1 may be named pLVAD (percutaneous left ventricle assisted device).

FIG. 2 illustrates an extra corporeal circular blood flow circuitry 206 comprising a dual lumen cannula 210 carrying a cage arrangement 216 near at least one outlet port and a blood pump P2. Cannula 210 is inserted through right internal jugular vein IJV, superior vena cava SVC, right atrium RA, transseptal, i.e. through the septum between right atrium RA and left atrium LA, into left atrium LA. Then, from left atrium LA through mitral valve MVa, left ventricle LV, through aortic valve AVa into ascending aorta aAO. A guide wire (not shown) may be used to guide cannula 210 to its final position. Alternatively, cannula 210 may be inserted through the right subclavian vein.

Blood is withdrawn by suction from left atrium LA through an outer lumen of cannula 210, see arrows 260, 262.

Blood is pumped into ascending aorta aAO through an inner lumen of cannula 210, see arrows 270, 272. Blood may be pumped in in a pulsed mode, preferably every time aortic valve AVa is closed. During the diastole, i.e. the heart refills with blood, there may be a first ejection of blood and during systole, i.e. contraction, there may be a normal or second ejection of blood out of the outlet holes of inner lumen of cannula 210. Alternatively, blood is only ejected during the systole.

Further to FIG. 2, a tube 220 is connected to a proximal end of the outer lumen of cannula 210 and to an inlet of pump P2. Pump P2 may be a peristaltic pump or centrifugal pump or another kind of pump, for instance a membrane pump. A tube 230 is connected to an outlet of pump P2 and to the proximal end of the inner lumen of cannula 210.

Pump P2 may be an electrically driven pump. There may be a control unit that controls the pumping performance, for instance depending on an ECG (electrocardiography) signal. Tubes 220, 230 may be made of a flexible material or of a more rigid material. Alternatively, pump P2 may be operated in continuous mode. Circuitry 206 may further include one or more blood filter units or units for dialysis of blood.

There may be a group of inlet holes 252 within the sidewall of the outer lumen within an intermediate portion of cannula 210, especially within an inlet portion 250 of cannula 210. Inlet holes 252 may be arranged circumferentially on all sides of cannula 210. Inlet holes 252 may be arranged at a location of cannula 210 that is within left atrium LA if cannula 210 is arranged in place as shown in FIG. 2. The outer lumen of cannula 210 may or may not extend distally beyond inlet holes 252. Furthermore, it is possible to reduce the outer diameter of cannula 210 distally beyond inlet portion 250.

Cage arrangement 216 is one possible example. Other possible examples are described below with reference to FIGS. 16 to 20. Cage arrangement 216 may comprise for instance between 6 to 12 flexible wires, beams or bars. There may be for instance 8 cage wires 218 that span a sphere. The sphere prevents that the side wall of the ascending aorta aAO covers one of the holes of tip 214. However, this is also prevented by the blood that is pumped out of distal end 212. Furthermore, cage arrangement 216 fixes distal end 212 of cannula 210 within ascending aorta aAO. Thus, it is not possible that cannula 210 slides back through aortic valve AVa into left ventricle LV.

The patient is able to walk because there are no cannulas in his legs or his groin. Furthermore, circuitry 206 supports the left part of heart H. The arrangement shown in FIG. 2 may be named pLVAD DL (percutaneous left ventricle assisted device dual lumen). The dual lumen cannula may be formed as shown. Alternatively, a dual lumen cannula may be used that is described in more detail below with regard to FIGS. 12 to 15.

FIG. 3 illustrates an extra corporeal circular blood flow circuitry 306 comprising a dual lumen cannula 310 carrying a cage arrangement 316 near at least one outlet port, a blood pump P3 and an oxygenator OXY3. Cannula 310 is inserted endovascularly through the right internal jugular vein IJV, superior vena cava SVC, right atrium RA, transseptal, i.e. through the septum between right atrium RA and left atrium LA, into left atrium LA. Then, from left atrium LA through mitral valve MVa, left ventricle LV, through aortic valve AVa into ascending aorta aAO. A guide wire (not shown) may be used to guide cannula 310 to its final position. Alternatively, cannula 310 may be inserted through the right subclavian vein.

Blood is withdrawn by suction from right atrium RA through an outer lumen of cannula 310, see arrows 360, 362.

Blood is pumped into the ascending aorta aAO through an inner lumen of cannula 310, see arrows 370, 372. Blood may be pumped in in a pulsed mode, preferably every time aortic valve AVa is closed. During the diastole, i.e. the heart refills with blood, there may be a first ejection of blood and during systole, i.e. contraction, there may be a normal or second ejection of blood out of the outlet holes of inner lumen of cannula 310. Alternatively, blood is only ejected during the systole. Alternatively, a continuous blood flow may be generated.

With reference further to FIG. 3, a tube 320 is connected to a proximal end of the outer lumen of cannula 210 and to an inlet of pump P3. Pump P3 may be a peristaltic pump or centrifugal pump or another kind of pump, for instance a membrane pump. A tube 340 is connected to an outlet of pump P2 and to the inlet of an oxygenator device OXY3. Oxygenator device OXY3 is used to enrich the oxygen content of the blood. Alternatively or additionally, oxygenator device OXY3 may also reduce carbon dioxide within blood. An outlet of Oxygenator OXY3 is connected to the proximal end of inner lumen of cannula 310 by a tube 330.

Pump P3 may be an electrically driven pump. There may be a control unit that controls the pumping performance, for instance depending on an ECG (electrocardiography) signal. Alternatively, pump P3 may be operated in continuous mode. Tubes 220, 230 may be made of a flexible material or of a more rigid material. Circuitry 306 may further include one or more blood filter units or units for dialysis of blood.

There may be a group of inlet holes 352 within the sidewall of the outer lumen of cannula 310 within an intermediate portion of cannula 310, especially within an inlet portion 350 of cannula 310. Inlet holes 352 may be arranged circumferentially on all sides of cannula 310. Inlet holes 352 may be arranged at a location of cannula 310 that is within right atrium RA if cannula 310 is arranged in place as shown in

FIG. 3. An optional second inlet portion 354 may be arranged more distally than inlet portion 350. However, the inlet holes of second inlet portion 354 may also be arranged at a location of cannula 310 that is within right atrium RA if cannula 310 is arranged in place as shown in FIG. 3. The outer lumen of cannula 310 may or may not extend distally beyond the inlet holes 252. Furthermore, it is possible to reduce the outer diameter of cannula 310 distally beyond inlet portion 350 or beyond inlet portion 354.

Cage arrangement 316 is one possible example. Other possible examples are described below with reference to FIGS. 16 to 20. Cage arrangement 316 may comprise for instance between 6 to 12 flexible wires, beams or bars. There may be for instance 8 cage wires 318 that span a sphere. The sphere prevents that the side wall of the ascending aorta aAO covers one of the holes of tip 314. However, this is also prevented by the blood that is pumped out of distal end 312. Furthermore, cage arrangement 316 fixes distal end 312 of cannula 310 within ascending aorta aAO. Thus, it is not possible that cannula 310 slides back through aortic valve AVa into left ventricle LV.

The patient is able to walk because there are no cannulas in his legs or his groin. Furthermore, circuitry 306 supports both the left part and the right part of heart H. The arrangement shown in FIG. 3 may be named as pBiVAD DL (percutaneous bi ventricle assist device dual lumen). Dual lumen cannula 310 may be formed as shown. Alternatively, a dual lumen cannula may be used that is described in more detail below with regard to FIGS. 12 to 15.

FIG. 4 illustrates a transcaval extra corporeal circular blood flow circuitry 406 comprising a single lumen cannula 410 carrying a cage arrangement 416 near at least one inlet port, a blood pump P4 and a second single lumen cannula 440 that has at least one outlet port in the artery of the lower trunk. First single lumen cannula 410 is inserted through the right internal jugular vein IJV, superior vena cava SVC, right atrium RA, trans-septal, i.e. through the septum between right atrium RA and left atrium LA, into left atrium LA. A guide wire (not shown) may be used to guide cannula 410 to its final position. Alternatively, cannula 410 may be inserted through the right subclavian vein. Blood is withdrawn by suction from left atrium LA through cannula 410, see arrows 460, 462.

Cannula 440 is inserted through the right femoral vein into the common femoral vein CFV and then transcaval via a transcaval passage 480 into common femoral artery CFA where blood is injected in a retrograde fashion into common femoral artery CFA, see arrows 470 and 472. Means may be used in order to support the vein and/or the artery openings that are part of transcaval passage 480. These means may be left within body 100 after removing cannula 440 for further uses. An example for such means is a fixation set that is available within the market.

Body 100 comprises a head 102 and a trunk 104. Heart H of a patient is located within trunk 104. The patient may be a male or female adult or a child. The description of the heart is given above with regard to FIG. 1. This description is valid for all FIGS. 1 to 15.

An optional inlet tip 414 may be mounted on distal end 412 of cannula 410. Inlet tip 414 may comprise a plurality of inlet holes 415 in its side wall. Additionally, there may be a hole within the distal end of inlet tip 414. The sum of the cross-section areas of the holes of tip 414 may be greater than the inner cross section area of cannula 410 at its distal end 112, for instance greater than twice the area or the triple of the area. This means that blood can be removed even if one or more of the inlet holes 415 in inlet tip 414 is or are clogged.

However, in other embodiments no separate inlet tip 414 is used. Thus, there is only one inlet hole at distal end 412 of cannula 410. This single inlet hole would be surrounded by cage arrangement 416.

Cage arrangement 416 is one possible example. Other possible examples are described below with reference to FIGS. 16 to 20. Cage arrangement 416 may comprise for instance between 6 to 12 flexible wires, beams or bars. There may be for instance 8 cage wires 118 that span a sphere. The sphere prevents that the side wall of left atrium LA covers one of inlet holes 415 of tip 414. Furthermore, cage arrangement 416 fixes distal end 412 of cannula 410 to the septum. Thus, it is not possible that cannula 410 slides back into right atrium RA.

With reference further to FIG. 4, a tube 420 is connected to a proximal end of cannula 410 and to an inlet of pump P4. Pump P4 may be a peristaltic pump or centrifugal pump or another kind of pump. A tube 430 is connected to an outlet of pump P4 and to the proximal end of cannula 440. Pump P4 may be an electrically driven pump. There may be a control unit that controls the pumping performance, for instance depending on an ECG (electrocardiography) signal. Pump P4 may be operated in pulsed or continuous mode.

Tubes 420, 430 may be made of a flexible material or of a more rigid material. The circuitry 406 may further include one or more blood filter units or units for dialysis of blood.

Cannula 440 may comprise an optional outlet tip 450 that has the same structure as inlet tip 414 of cannula 410. This means that outlet tip 450 may comprise a plurality of outlet holes 452 in its side wall and/or on its distal end. Additionally, cannula 440 may comprise a cage arrangement on its distal end 442. The cage arrangement may be formed as described above for instance for cage arrangement 116 or cage arrangement 546, see FIG. 5 and corresponding description.

No extra care has to be taken because cannula 440 is inserted first into a vein in which there is comparably low blood pressure. However, the transcaval passage 480 has to be handled with care because blood pressure is much higher in an artery compared to blood pressure in a vein. Furthermore, blood flow in a vein is continuously but blood flow in an artery is pulsed. Pulsed mode of the pump is not necessary because of the retrograde infusion.

However, pump P4 may be operated in a pulsed mode. Control may be performed depending on the rhythm of the heartbeat. A sensor may be used to detect the heartbeat, especially an electronic sensor. If the heart is in a diastolic state the counter pressure against infusion of blood into artery CFA may be weak.

Retrograde infusion of blood may not be as advantageous as antegrade infusion because of water divides of the lymphatic system that may be formed and because of the forming of turbulences. The formation of thrombus may be facilitated by retrograde infusion. The arrangement shown in FIG. 4 may be used for patients without lung problems. Furthermore, circuitry 406 supports the left part of heart H of the patient. The arrangement shown in FIG. 1 may be named pLVAD transcaval (percutaneous left ventricle assisted device).

FIG. 5 illustrates a transcaval extra corporeal circular blood flow circuitry 506 comprising a single lumen cannula 510 carrying a cage arrangement 516 near at least one inlet port, a blood pump P5 and a second single lumen cannula 540 that has at least one outlet port in the artery of the lower trunk 104. First single lumen cannula 510 is inserted through right internal jugular vein IJV, superior vena cava SVC, right atrium RA, transseptal, i.e. through the septum between right atrium RA and left atrium LA, into left atrium LA. A guide wire (not shown) may be used to guide cannula 510 to its final position. Alternatively, cannula 510 may be inserted through the right subclavian vein. Blood is withdrawn by suction from left atrium LA through cannula 510, see arrows 560, 562.

Cannula 540 is inserted through the left internal jugular vein IJV, superior vena cava SVC, right atrium RA, inferior vena cava IVC and then transcaval via a transcaval passage 580 into common femoral artery CFA where blood is injected in a retrograde fashion into common femoral artery CFA, see arrow 570. Means may be used in order to support the vein and/or the artery openings that are part of transcaval passage 580. These means may be left within body 100 after removing cannula 540 for further uses. An example for such means is a fixation set that is available within the market.

An optional inlet tip 514 may be mounted on distal end 512 of cannula 510. Inlet tip 514 may comprise a plurality of inlet holes 515 in its side wall. Additionally, there may be a hole within the distal end of inlet tip 514. The sum of the cross-section areas of the holes of tip 514 may be greater than the inner cross section area of cannula 510 at its distal end 512, for instance greater than twice the area or the triple of the area. This means that blood can be removed even if one or more of the inlet holes 515 in inlet tip 514 is or are clogged.

However, in other embodiments no separate inlet tip 514 is used. Thus, there is only one inlet hole at the distal end 512 of cannula 510. This single inlet hole would be surrounded by cage arrangement 516.

Cage arrangement 516 is one possible example. Other possible examples are described below with reference to FIGS. 16 to 20. Cage arrangement 516 may comprise for instance between 6 to 12 flexible wires, beams or bars. There may be for instance 8 cage wires 518 that span a sphere. The sphere prevents that the side wall of left atrium LA covers one of inlet holes 515 of tip 514. Furthermore, cage arrangement 516 fixes distal end 512 of cannula 510 to the septum. Thus, it is not possible that cannula 510 slides back into right atrium RA.

Further to FIG. 5, a tube 520 is connected to a proximal end of cannula 510 and to an inlet of pump P5. Pump P5 may be a peristaltic pump or centrifugal pump or another kind of pump, for instance a membrane pump. A tube 530 is connected to an outlet of pump P5 and to the proximal end of cannula 540. Pump P5 may be an electrically driven pump. There may be a control unit that controls the pumping performance, for instance depending on an ECG (electrocardiography) signal. Pump P5 may be operated in pulsed or continuous mode.

Tubes 520, 530 may be made of a flexible material or of a more rigid material. Circuitry 506 may further include one or more blood filter units or units for dialysis of blood.

Cannula 540 may comprise an optional outlet tip 550 that may have the same structure as inlet tip 514 of cannula 510. This means that outlet tip 550 may comprise a plurality of outlet holes 452 in its side wall and/or on its distal end. Additionally, cannula 540 may have an optional cage arrangement 546 on its distal end 542.

Cage arrangement 546 is one possible example. Other possible examples are described below with reference to FIGS. 16 to 20. Cage arrangement 546 may comprise for instance between 6 to 12 flexible wires, beams or bars. There may be for instance 8 cage wires 548 that span a sphere. The sphere prevents that the side wall of common femoral artery CFA covers one of outlet holes 552 of optional outlet tip 550. Furthermore, cage arrangement 546 fixes distal end 542 of cannula 540 within common femoral artery CFA and allows antegrade blood flow of blood coming from heart H and/or from outlet holes 552.

No extra care has to be taken because cannula 540 is inserted first into a vein in which there is comparably low blood pressure. However, transcaval passage 580 has to be handled with care because blood pressure is much higher in an artery compared to blood pressure in a vein. Furthermore, blood flow from a vein is continuously but blood flow in an artery is pulsed. Pulsed mode of the pump is not necessary because of the retrograde infusion.

However, pump P5 may be operated in a pulsed mode. Control may be performed depending on the rhythm of the heartbeat of heart H. A sensor may be used to detect the heartbeat, especially an electronic sensor. If heart H is in a diastolic state the counter pressure against infusion of blood into common femoral artery CFA may be weak.

Retrograde infusion of blood may not be as advantageous as antegrade infusion because of water divides of the lymphatic system and of the forming of turbulences. The formation of thrombus may be facilitated by retrograde infusion.

The arrangement shown in FIG. 5 may be used for patients without lung problems. Furthermore, circuitry 506 supports the left part of the heart H of the patient. Mobility of the patient is possible because no cannulas in femoral veins or arteries are used. Compared with FIG. 4, the arrangement of FIG. 5 allows blood injection more central within body 100. The arrangement shown in FIG. 5 may be named pLVAD transcaval (percutaneous left ventricle assisted device).

In other embodiments it is possible to insert cannula 510 through the left internal jugular vein IJV to the left atrium LA as described above and cannula 540 through the right internal jugular vein IJV into the common femoral artery CFA.

### B) LUNG ASSIST

FIG. 6 illustrates an extra corporeal circular blood flow circuitry 606 comprising two single lumen cannulas 610 and 620 and a carbon dioxide removal device CO2R6 but no pump. Single lumen cannula 610 carries a cage arrangement 616 near at least one inlet port that is arranged in pulmonary artery PA. Second single lumen cannula 640 has at least one outlet port within left atrium LA.

First single lumen cannula 610 is inserted through right internal jugular vein IJV, superior vena cava SVC, right atrium RA, right ventricle RV, through pulmonary valve PVa into pulmonary artery PA. A guide wire (not shown) may be used to guide cannula 610 to its final position. Alternatively, cannula 610 may be inserted through the right subclavian vein and then along the same way as described above. Blood is withdrawn by suction from pulmonary artery PA through cannula 610, see arrow 660. A part of the blood that comes from right ventricle RV is pumped by heart H into pulmonary artery PA and is enriched in the lung with oxygen. Removal of carbon dioxide may be necessary for instance for patients that have chronic obstructive pulmonary disease (COPD) or cystic fibrosis.

Cannula 640 is inserted through left internal jugular vein IJV, superior vena cava SVC, right atrium RA, trans-septal, i.e. through the septum between right atrium RA and left atrium LA, into left atrium LA. A guide wire (not shown) may be used to guide cannula 640 to its final position. Alternatively, cannula 610 may be inserted through the right subclavian vein.

An optional inlet tip 614 may be mounted on distal end 612 of cannula 610. Inlet tip 614 may comprise a plurality of inlet holes 615 in its side wall. Additionally, there may be a hole within the distal end of inlet tip 614. The sum of the cross-section areas of the holes of tip 614 may be greater than the inner cross section area of cannula 610 at its distal end 612, for instance greater than twice the area or the triple of the area. This means that blood can be removed even if one or more of inlet holes 615 in inlet tip 614 is or are clogged.

However, in other embodiments no inlet tip 614 is used. Thus, there is only one inlet hole at the distal end 612 of cannula 610. This single inlet hole would be surrounded by cage arrangement 616.

Cage arrangement 616 is one possible example. Other possible examples are described below with reference to FIGS. 16 to 20. Cage arrangement 616 may comprise for instance between 6 to 12 flexible wires, beams or bars. There may be for instance 8 cage wires 618 that span a sphere. The sphere prevents that the side wall of left atrium LA covers one of inlet holes 615 of tip 614. Furthermore, cage arrangement 616 fixes distal end 612 of cannula 510 to pulmonary artery PA. Thus, it is not possible that cannula 510 slides back through pulmonary valve PV into right ventricle RV.

Further to FIG. 6, a tube 620 is connected to a proximal end of cannula 610 and to an inlet of carbon dioxide removal device CO2R6 that removes carbon dioxide from the blood. A pump may not be necessary but may be used in another embodiment. A tube 630 is connected to an outlet of carbon dioxide removal device CO2R6 and to the proximal end of cannula 640. Carbon dioxide removal device CO2R6 may contain a semipermeable membrane.

Tubes 620, 630 may be made of a flexible material or of a more rigid material. Circuitry 606 may further include one or more blood filter units or units for dialysis of blood. However, the natural blood pressure may not be sufficient to press the blood also through a filter device without using a pump.

Cannula 640 may comprise an optional outlet tip 650 that may have the same structure as the inlet tip 614 of cannula 610. This means that outlet tip 650 may comprise a plurality of outlet holes 652 in its side wall and/or on its distal end. Additionally, cannula 640 may have an optional cage arrangement 646 on its distal end 642. Blood with less carbon dioxide is injected into the left atrium LA through cannula 640 and mixes with oxygen rich blood that comes through the pulmonary veins from the lung.

Cage arrangement 646 is one possible example. Other possible examples are described below with reference to FIGS. 16 to 20. Cage arrangement 646 may comprise for instance between 6 to 12 flexible wires, beams or bars. There may be for instance 8 cage wires 648 that span a sphere. The sphere and also the expelled blood prevent that the side wall of left atrium LA covers one of the outlet holes 652 of optional outlet tip 650. Furthermore, cage arrangement 646 fixes distal end 642 of cannula 640 within left atrium LA.

No extra care has to be taken because both cannulas 510 and 540 are inserted into veins in which there is comparably low blood pressure.

Antegrade infusion is performed that has many advantages, i.e. no forming of water divides of the lymphatic system and less forming of turbulences. The formation of thrombus may be prevented by antegrade infusion.

The arrangement shown in FIG. 6 may be used for patients with lung problems. Mobility of the patient is possible because no cannulas in femoral veins or arteries are used. The arrangement shown in FIG. 6 may be named pECLA (percutaneous left extra corporeal lung assist).

In other embodiments it is possible to insert cannula 610 through left internal jugular vein IJV/left subclavian vein to pulmonary artery PA as described above and cannula 640 through right internal jugular vein IJV/right subclavian vein to left atrium LA.

In another embodiment a pump is connected in series with carbon dioxide removal device CO2R6. This allows to remove more carbon dioxide from the blood, for instance more than 30 percent compared to the content on the inlet of the carbon dioxide removal device CO2R6. This embodiment may be named ECCO₂R (extracorporeal CO₂ removal).

In a further embodiment an oxygenator device is used instead of carbon dioxide removal device CO2R6 and preferably a pump is connected in series with the oxygenator. The oxygenator device enriches the oxygen content in the blood and decreases the carbon dioxide content at the same time. This further embodiment may be named ECMO (extracorporeal membrane oxygenation).

FIG. 7 illustrates a transcaval extra corporeal lung assist circular blood flow circuitry 706 comprising a single lumen cannula 710 carrying a cage arrangement 716 near at least one inlet port, a carbon dioxide removal device CO2R7 and a single lumen cannula 740 that has at least one outlet port in the right atrium RA.

Cannula 710 is inserted through right internal jugular vein IJV, superior vena cava SVC, right atrium RA, inferior vena cava IVC and then transcaval via a transcaval passage 780 into common femoral artery CFA where blood with comparably high oxygen content is withdrawn from, see arrows 760, 762. A guide wire (not shown) may be used to guide cannula 710 to its final position. Alternatively, cannula 710 may be inserted through the right subclavian vein. Means may be used in order to support the vein and/or the artery openings that are part of transcaval passage 780. These means may be left within body 100 after removing cannula 710 for further uses. An example for such means is a fixation set that is available within the market.

Single lumen cannula 740 is inserted through left internal jugular vein IJV, superior vena cava SVC into right atrium RA. A guide wire (not shown) may be used to guide cannula 710 to its final position. Alternatively, cannula 710 may be inserted through right subclavian vein. Blood with reduced carbon dioxide content is ejected into the right atrium RA through cannula 740, see arrows 770, 772. This blood is then pumped by heart H through right ventricle RV and pulmonary artery PA, see FIG. 6, to lung L of the patient having body 100.

An optional inlet tip 714 may be mounted on distal end 712 of cannula 710. Inlet tip 714 may comprise a plurality of inlet holes 715 in its side wall. Additionally, there may be a hole within the distal end of inlet tip 714. The sum of the cross section areas of the holes of tip 714 may be greater than the inner cross section area of cannula 710 at its distal end 712, for instance greater than twice the area or the triple of the area. This means that blood can be removed even if one or more of the inlet holes 715 in inlet tip 714 is or are clogged.

However, in other embodiments no inlet tip 714 is used. Thus, there is only one inlet hole at distal end 712 of cannula 710. This single inlet hole would be surrounded by cage arrangement 716.

Cage arrangement 716 is one possible example. Other possible examples are described below with reference to FIGS. 16 to 20. Cage arrangement 716 may comprise for instance between 6 to 12 flexible wires, beams or bars. There may be for instance 8 cage wires 718 that span a sphere. The sphere prevents that the side wall of common femoral artery CFA covers one of inlet holes 715 of tip 714. Furthermore, cage arrangement 716 fixes distal end 712 of cannula 710 to common femoral artery CFA. Thus it is not possible that cannula 710 slides back into transcaval passage 780.

With reference further to FIG. 7, a tube 720 is connected to a proximal end of cannula 710 and to an inlet of carbon dioxide removal device CO2R7. Carbon dioxide removal device CO2R7 may comprise a semipermeable membrane. A tube 730 is connected to an outlet of carbon dioxide removal device CO2R7 and to the proximal end of cannula 840.

Tubes 720, 730 may be made of a flexible material or of a more rigid material. Circuitry 706 may further include one or more blood filter units or units for dialysis of blood. However, an additional pump may be necessary if a filter unit/dialysis unit is used.

Cannula 740 may comprise an optional outlet tip 750 that may have the same structure as inlet tip 714 of cannula 710. This means that outlet tip 750 may comprise a plurality of outlet holes 752 in its side wall and/or on its distal end. Additionally, cannula 740 may have an optional cage arrangement 746 on its distal end 742.

Cage arrangement 746 is one possible example. Other possible examples are described below with reference to FIGS. 16 to 20. Cage arrangement 746 may comprise for instance between 6 to 12 flexible wires, beams or bars. There may be for instance 8 cage wires 548 that span a sphere. The sphere prevents that the side wall of common femoral artery CFA covers one of outlet holes 752 of optional outlet tip 750. Furthermore, cage arrangement 746 fixes distal end 742 of cannula 740 within right atrium RA.

No extra care has to be taken because both cannulas 710 and 740 are inserted first into a vein in which there is comparably low blood pressure. However, transcaval passage 780 has to be handled with care because blood pressure is much higher in an artery compared to blood pressure in a vein. Furthermore, blood flow from a vein is continuously but blood flow in an artery is pulsed.

Antegrade infusion is performed that has many advantages, i.e. no forming of water divides of the lymphatic system may occur and less forming of turbulences may be present. The formation of thrombus may be prevented by antegrade infusion.

The arrangement shown in FIG. 7 may be used for patients with lung problems. Mobility of the patient is possible because no cannulas in femoral veins or arteries are used. The arrangement shown in FIG. 7 may be named pECLA (percutaneous left extra corporeal lung assist) transcaval.

In other embodiments it is possible to insert cannula 710 through left internal jugular vein UV/left subclavian vein to common femoral artery CFA as described above and cannula 740 through right internal jugular vein IJV into right atrium RA.

In another embodiment a pump is connected in series with carbon dioxide removal device CO2R7. This allows to remove more carbon dioxide from the blood, for instance more than 30 percent compared to the content on the inlet of the carbon dioxide removal device CO2R7.

In a further embodiment an oxygenator device is used instead of carbon dioxide removal device CO2R7 and preferably a pump is connected in series with the oxygenator. The oxygenator device enriches the oxygen content in the blood and decreases the carbon dioxide content at the same time.

FIG. 8 illustrates a transcaval transseptal extra corporeal lung assist circular blood flow circuitry 806 comprising a single lumen cannula 810 carrying a cage arrangement 816 near at least one inlet port, a carbon dioxide removal device CO2R8 and a single lumen cannula 840 that has at least one outlet port in the left atrium LA.

Cannula 810 is inserted through the right internal jugular vein IJV, superior vena cava SVC, right atrium RA, inferior vena cava IVC and then transcaval via a transcaval passage 880 into common femoral artery CFA where blood with comparably high oxygen content is withdrawn from, see arrows 860, 862. A guide wire (not shown) and/or snares may be used to guide cannula 810 to its final position. Alternatively, cannula 810 may be inserted through the right subclavian vein. Means may be used in order to support the vein and/or the artery openings that are part of transcaval passage 880. These means may be left within body 100 after removing cannula 810 for further uses. An example for such means is a fixation set that is available within the market.

Single lumen cannula 840 is inserted through the left internal jugular vein IJV, superior vena cava SVC, right atrium RA, trans-septal, i.e. through the septum between right atrium RA and left atrium LA, into left atrium LA. A guide wire (not shown) may be used to guide cannula 810 to its final position. Alternatively, the cannula 810 may be inserted through the right subclavian vein. Blood with reduced carbon dioxide content is ejected into left atrium LA through cannula 840, see arrow 870. This blood is then pumped by heart H through right ventricle RV and pulmonary artery PA, see FIG. 6, to lung L of the patient having body 100.

An optional inlet tip 814 may be mounted on distal end 812 of cannula 810. Inlet tip 814 may comprise a plurality of inlet holes 815 in its side wall. Additionally, there may be a hole within the distal end of inlet tip 814. The sum of the cross section areas of the holes of tip 814 may be greater than the inner cross section area of cannula 810 at its distal end 812, for instance greater than twice the area or the triple of the area. This means that blood can be removed even if one or more of the inlet holes 815 in inlet tip 814 is or are clogged.

However, in other embodiments no inlet tip 814 is used. Thus, there is only one inlet hole at distal end 812 of cannula 810. This single inlet hole would be surrounded by cage arrangement 816.

Cage arrangement 816 is one possible example. Other possible examples are described below with reference to FIGS. 16 to 20. Cage arrangement 816 may comprise for instance between 6 to 12 flexible wires, beams or bars. There may be for instance 8 cage wires 818 that span a sphere. The sphere prevents that the side wall of common femoral artery CFA covers one of inlet holes 815 of tip 814. Furthermore, cage arrangement 816 fixes distal end 812 of cannula 710 within common femoral artery CFA. Thus, it is not possible that cannula 810 slides unintentionally back into transcaval passage 880.

Further to FIG. 8, a tube 820 is connected to a proximal end of cannula 810 and to an inlet of carbon dioxide removal device CO2R8. Carbon dioxide removal device CO2R8 may comprise a semipermeable membrane. A tube 830 is connected to an outlet of carbon dioxide removal device CO2R8 and to the proximal end of cannula 840.

Tubes 820, 830 may be made of a flexible material or of a more rigid material. The circuitry 806 may further include one or more blood filter units or units for dialysis of blood. However, an additional pump may be necessary if a filter unit/dialysis unit is used.

Cannula 840 may comprise an optional outlet tip 850 that may have the same structure as the inlet tip 814 of cannula 810. This means that outlet tip 850 may comprise a plurality of outlet holes 852 in its side wall and/or on its distal end. Additionally, cannula 840 may have an optional cage arrangement 846 on its distal end 842.

Cage arrangement 846 is one possible example. Other possible examples are described below with reference to FIGS. 16 to 20. Cage arrangement 846 may comprise for instance between 6 to 12 flexible wires, beams or bars. There may be for instance 8 cage wires 848 that span a sphere. The sphere and the ejected blood prevents that the side wall of common femoral artery CFA covers one of outlet holes 852 of optional outlet tip 850. Furthermore, cage arrangement 846 fixes distal end 842 of cannula 840 within left atrium LA.

No extra care has to be taken because both cannulas 810 and 840 are inserted first into a vein in which there is comparably low blood pressure. However, transcaval passage 880 has to be handled with care because blood pressure is much higher in an artery compared to blood pressure in a vein. Furthermore, blood flow from a vein is continuously but blood flow in an artery is pulsed.

Antegrade infusion is performed that has many advantages, i.e. no forming of water divides of the lymphatic system and less forming of turbulences. The formation of thrombus may be prevented by antegrade infusion.

The arrangement shown in FIG. 8 may be used for patients with lung problems. Mobility of the patient is possible because no cannulas in femoral veins or arteries are used. The arrangement shown in FIG. 8 may be named pECLA (percutaneous left extra corporeal lung assist) transcaval transseptal.

In other embodiments it is possible to insert cannula 810 through left internal jugular vein IJV/left subclavian vein to common femoral artery CFA as described above and cannula 840 through right internal jugular vein IJV into right atrium RA.

In another embodiment a pump is connected in series with carbon dioxide removal device CO2R8. This allows to remove more carbon dioxide from the blood, for instance more than 30 percent compared to the content on the inlet of the carbon dioxide removal device CO2R8.

In a further embodiment an oxygenator is used instead of carbon dioxide removal device CO2R8 and preferably a pump is connected in series with the oxygenator device. The oxygenator device enriches the oxygen content in the blood and decreases the carbon dioxide content at the same time.

### C) LUNG PERFUSION

An isolation of the lung L is reached together with heart assist of heart H at the same moment for the circuitries that use percutaneous in-vivo lung perfusion (pIVLP). Thus, isolated perfusion and/or treatment of lung diseases is enabled, especially antegrade and/or retrograde, preferably also with switching between antegrade and retrograde or between retrograde and antegrade. However, if only a part of the lung is treated, the other part may function normal. There may be a lobe dedicated treatment or treatment of only a part of a lobe. This may allow to treat the lung L without heart H assist/support and or without lung support, e.g. without external blood oxygenation and/or without external carbon dioxide (CO₂) removal. Alternatively, partially or full heart H assist and/or lung L assist may be used even if only a part of the lung is treated, for instance.

FIG. 9 illustrates an extra corporeal lung perfusion circular blood flow circuitry 906 comprising two single lumen cannulas 910 and 940, a pump P9 and a further device D9. Single lumen cannula 910 carries a cage arrangement 916 near at least one inlet port that is arranged in left atrium LA. Second single lumen cannula 940 has at least one outlet port within pulmonary artery PA. Circuitry 906 is a more theoretical embodiment because in addition a heart assist would be necessary. However, there are many ways to realize such a heart assist. One possibility is described below with reference to FIG. 10.

Cannula 910 is inserted through the left internal jugular vein IJV, superior vena cava SVC, right atrium RA, trans-septal, i.e. through the atrial septum AS between right atrium RA and left atrium LA, into left atrium LA. A guide wire (not shown) may be used to guide cannula 910 to its final position. Alternatively, cannula 910 may be inserted through the right subclavian vein. Almost the whole blood that enters left atrium LA through the left and right pair of pulmonary veins PV may be taken in by cannula 910, see arrow 960, using a membrane 919 that is explained in more detail below.

The second single lumen cannula 940 is inserted through the right internal jugular vein IJV, superior vena cava SVC, right atrium RA, right ventricle RV, through pulmonary valve PVa into pulmonary artery PA. A guide wire (not shown) may be used to guide cannula 940 to its final position. Alternatively, cannula 940 may be inserted through the right subclavian vein and then along the same way as described above. Almost the whole blood that comes out of cannula 940 is injected into pulmonary artery PA, see arrow 970, using a membrane 949 that is explained in more detail below. Device D9 may be an injection device that injects a medicament or a treatment substance, for instance for treating lung cancer.

An optional inlet tip 914 may be mounted on distal end 912 of cannula 910. Inlet tip 914 may comprise a plurality of inlet holes 915 in its side wall. Additionally, there may be a hole within the distal end of inlet tip 914. The sum of the cross section areas of the holes of tip 914 may be greater than the inner cross section area of cannula 910 at its distal end 912, for instance greater than twice the area or the triple of the area. This means that blood can be removed even if one or more of inlet holes 915 in inlet tip 914 is or are clogged.

However, in other embodiments no inlet tip 914 is used. Thus, there is only one inlet hole at distal end 912 of cannula 910. This single inlet hole would be surrounded by cage arrangement 916. Using a cannula without a separate tip allows high flow rates of a fluid that is drained into the cannula 910. The cage arrangement 916 prevents that a wall of left atrium LA is sucked into the hole of cannula 910.

Cage arrangement 916 is one possible example. Other possible examples are described below with reference to FIGS. 16 to 20. Cage arrangement 916 may comprise for instance between 6 to 12 flexible wires, beams or bars. There may be for instance 8 cage wires 918 that span a sphere. The sphere prevents that the side wall of the left atrium LA covers one of inlet holes 915 of tip 914. Furthermore, cage arrangement 916 fixes distal end 912 of cannula 910 to the atrial septum AS. Thus, it is not possible that cannula 910 slides back through the atrial septum into right atrium RA.

Membrane 919 may cover only one half of cage arrangement 916, e.g. a half that is defined by two cage wires 918 that are arranged opposite to each other or nearly opposite. Examples of membranes that may be used on cage arrangement 916 are described below with reference to FIGS. 16 to 20.

Further to FIG. 9, a tube 920 is connected to a proximal end of cannula 910 and to an inlet of pump P9. An outlet of pump P9 may be connected to an inlet of device D9. A tube 930 is connected to an outlet of device D9 and to the proximal end of cannula 940. Device D9 may be used for instance for injecting a drug or medicament or treatment substance into the lung of the patient.

Tubes 920, 930 may be made of a flexible material or of a more rigid material. The circuitry 906 may further include one or more blood filter units or units for dialysis of blood.

Cannula 940 may comprise an optional outlet tip 950 that may have the same structure as inlet tip 914 of cannula 910. This means that outlet tip 950 may comprise a plurality of outlet holes 952 in its side wall and/or on its distal end. Additionally, cannula 940 may have an optional cage arrangement 946 on its distal end 942.

However, in other embodiments no outlet tip 950 is used. Thus, there is only one inlet hole at distal end 942 of cannula 940. This single inlet hole may be surrounded by cage arrangement 946.

Cage arrangement 946 is one possible example. Other possible examples are described below with reference to FIGS. 16 to 20. Cage arrangement 946 may comprise for instance between 6 to 12 flexible wires, beams or bars. There may be for instance 8 cage wires 948 that span a sphere. The sphere and also the expelled blood prevent that the side wall of pulmonary artery PA covers one of outlet holes 952 of optional outlet tip 950. Furthermore, cage arrangement 946 fixes distal end 942 of cannula 940 within pulmonary artery PA.

Membrane 949 may cover only one half of cage arrangement 946, e.g. a half that is between the distal end of cannula 940 and the mid of cage wires 948. Examples of membranes that may be used on cage arrangement 946 are described below with reference to FIG. 16.

No extra care has to be taken because both cannulas 910 and 940 are inserted into veins in which there is comparably low blood pressure compared to the blood pressure in arteries. Antegrade infusion is performed into pulmonary artery PA that has many advantages because it corresponds to the natural direction of blood flow in lung L of the patient.

The arrangement shown in FIG. 9 may be used for patients with lung problems. Mobility of the patient is possible because no cannulas are used in femoral veins or arteries. The arrangement shown in FIG. 9 may be named pIVLP (percutaneous in vivo lung perfusion).

In other embodiments it is possible to insert cannula 910 through right internal jugular vein IJV/right subclavian vein to left atrium LA as described above and cannula 940 through left internal jugular vein IJV/left subclavian vein to left atrium LA.

Alternatively, device D9 may be a CO₂ (carbon dioxide) removal device, an oxygenator. Furthermore, the pumping direction may be reversed, i.e. from antegrade to retrograde.

During treatment of the lung L it is possible that the patient inhales a medicament or treatment substance in order to promote the treatment by the substance or medicament that flows through the vessels of the lung L and through the tissue of the alveoli. The fluid flow within circuitry 906 may comprise blood as a carrier substance. Alternatively, other carrier substances may be used, for instance based on saline and/or on water.

FIG. 10 illustrates an extra corporeal retrograde lung perfusion circular blood flow circuitry 1006 comprising two dual lumen cannulas 1010 and 1040, two pumps P10a, P10b and an oxygenator device OXY10. Dual lumen cannula 1010 carries a cage arrangement 1016 near at least one inlet port that is arranged in pulmonary artery PA and an inlet portion 1090 that is arranged in right atrium RA. Second dual lumen cannula 1040 has at least one outlet port within ascending aorta aAO and an outlet portion 1084 in left atrium LA. The circuitry 1006 allows for instance the removal of thrombus from the lung L of patient. Alternatively, a chemotherapy of lung may be performed, a stem cell treatment or cleaning of the lung.

Dual lumen cannula 1010 is endovascularly inserted through right internal jugular vein IJV, superior vena cava SVC, right atrium RA, right ventricle RV, through pulmonary valve PVa into pulmonary artery PA. A guide wire (not shown) may be used to guide cannula 1010 to its final position. Alternatively, cannula 1010 may be inserted through the right subclavian vein and then along the same way as described above. Almost the whole blood that comes out of pulmonary artery PA is extracted into inner lumen of dual lumen cannula 1010, see arrow 1060, by using a membrane 1019 that is explained in more detail below. Other possibilities for insertion of a dual lumen cannula 1010 will be explained below, e.g. first insertion of outer lumen and then insertion of inner lumen.

Dual lumen cannula 1040 is endovascularly inserted through left internal jugular vein IJV, superior vena cava SVC, right atrium RA, trans-septal, i.e. through the septum between right atrium RA and left atrium LA, into left atrium LA. A guide wire (not shown) may be used to guide cannula 1040 to its final position. Alternatively, cannula 1040 may be inserted through the right subclavian vein. Almost the whole blood that exits the distal tip of the inner lumen of cannula 1040 is injected into ascending aorta aAO, see arrow 1070, by using a membrane 1049 that is explained in more detail below. Other possibilities for insertion of a dual lumen cannula 1040 will be explained below, e.g. first insertion of outer lumen and then insertion of inner lumen.

An optional inlet tip 1014 may be mounted on distal end 1012 of cannula 1010. Inlet tip 1014 may comprise a plurality of inlet holes 1015 in its side wall. Additionally, there may be a hole within the distal end of inlet tip 1014. The sum of the cross section areas of the holes of tip 1014 may be greater than the inner cross section area of cannula 1010 at its distal end 1012, for instance greater than twice the area or the triple of the area. This means that blood can be removed even if one or more of inlet holes 1015 in inlet tip 1014 is or are clogged.

However, in other embodiments no inlet tip 1014 is used. Thus, there is only one inlet hole at distal end 1012 of cannula 1010, i.e. at the proximal end of the cage arrangement. This single inlet hole would be surrounded by cage arrangement 1016. A single inlet hole may allow higher flow rates compared to inlet tip 1014 that comprises lateral inlet holes.

Cage arrangement 1016 is one possible example. Other possible examples are described below with reference to FIGS. 16 to 20. Cage arrangement 1016 may comprise for instance between 6 to 12 flexible wires, beams or bars. There may be for instance 8 cage wires 1018 that span a sphere. The sphere prevents that the side wall of left atrium LA covers one of inlet holes 1015 of tip 1014 or the single end hole if no inlet tip 1014 is used. Furthermore, cage arrangement 1016 fixes distal end 1012 of cannula 910 to pulmonary artery PA. Thus, it is not possible that cannula 1010 slides back through pulmonary valve PVa into left ventricle LV.

Membrane 1019 may cover only one half of cage arrangement 1016, e.g. a half that is between distal end 1012 of cannula 1010 and the mid of cage wires 1018. Examples of membranes that may be used on cage arrangement 1016 are described below with reference to FIG. 16.

Inlet portion 1090 may comprise a plurality of inlet holes that extend through the side wall of outer lumen of cannula 1010. Blood is extracted by suction from right atrium RA into outer lumen of cannula 1010, preferably all blood or nearly all (for instance more than 90 percent of volume) blood that comes into right atrium RA, see arrow 1092. An optional cage arrangement may be arranged at inlet portion 1090.

Further to FIG. 10, a tube 1020a is connected to a proximal end of inner lumen of cannula 1010 and to an inlet of pump P10a. An outlet of pump P10a may be connected to a proximal end of outer lumen of cannula 1040 using a tube 1030a.

A tube 1020b is connected to a proximal end of outer lumen of cannula 1010 and to an inlet of pump P10b. An outlet of pump P10b may be connected to oxygenator device OXY10. A tube 1030b is connected to an outlet of oxygenator device OXY10 and to the proximal end of inner lumen of cannula 1040. It is also possible to exchange the sequence of pump P10b and oxygenator device OXY10.

Pumps P10a, P10b may be peristaltic pumps, centrifugal pumps, membrane pumps or other kind of pumps. Oxygenator device OXY10 enriches blood with oxygen that comes out of right atrium RA and/or right ventricle RV (see inlet portion 1098 that is described in more detail below) and is then injected into ascending aorta aAO. Thus, the function of the lung is fulfilled by oxygenator device OXY10 during treatment of lung L and the right heart H is supported.

Tubes 1020a, 1020b, 1030a, 1030b may be made of a flexible material or of a more rigid material. The circuitry 1006 may further include one or more blood filter units or units for dialysis of blood. Furthermore, a device may be used within circuitry 1006 for instance for injecting a drug or medicament and/or a treatment substance into the lung L of the patient.

Cannula 1040 may comprise an optional outlet tip 1050 that may have the same structure as inlet tip 1014 of cannula 1010. This means that outlet tip 1050 may comprise a plurality of outlet holes 1052 in its side wall and/or on its distal end. Additionally, cannula 1040 may have an optional cage arrangement 1046 on its distal end 1042. If there is no outlet tip 1050 a single end-hole may be used at the distal end of cannula 1040, i.e. at the proximal end of cage arrangement 1046.

Cage arrangement 1046 is one possible example. Other possible examples are described below with reference to FIGS. 16 to 20. Cage arrangement 1046 may comprise for instance between 6 to 12 flexible wires, beams or bars. There may be for instance 8 cage wires 1048 that span a sphere. The sphere and also the expelled blood prevent that the side wall of the ascending aorta aAO covers one of outlet holes 1052 of optional outlet tip 1050. The sphere prevents that the injected blood damages the walls of the aorta AO, i.e. the “sand blasting effect” is prevented or mitigated even if no outlet tip 1050 is used. Furthermore, cage arrangement 1046 fixes distal end 1042 of cannula 1040 within ascending aorta aAO.

Membrane 1049 may cover only one half of cage arrangement 1046, e.g. a half that is between the distal end of cannula 1040 and the mid of cage wires 1048. Examples of membranes that may be used on cage arrangement 1048 are described below with reference to FIG. 16.

Outlet portion 1084 of cannula 1040 may comprises a plurality of outlet holes 1085 that extend through the sidewall of the outer lumen of cannula 1040. Blood is expelled through outlet portion 1084 into the pairs of left and right pulmonary veins PV, see arrow 1075. Blood flow to left ventricle LV is thereby prevented by using membrane 1089. Outlet portion 1084 may be surrounded by an optional cage arrangement 1086. Moreover, the sand blasting effect is prevented or mitigated. Furthermore, cage arrangement 1086 fixes outlet portion 1086 of cannula 1040 within left atrium LA.

Cage arrangement 1086 is one possible example. Other possible examples are described below with reference to FIGS. 16 to 20. Cage arrangement 1086 may comprise for instance between 6 to 12 flexible wires, beams or bars. There may be for instance 8 cage wires 1088 that span a sphere. The sphere and also the expelled blood prevent that the side wall of left atrium LA covers one of outlet holes 1085. Furthermore, cage arrangement 1086 fixes outlet portion 1086 of cannula 1040 within left atrium LA.

Membrane 1089 may cover only one half of cage arrangement 1086, e.g. a half that is defined by two cage wires 1088 that are arranged opposite to each other or nearly opposite. Examples of membranes that may be used on cage arrangement 1086 are described below with reference to FIG. 17.

In summary, the following blood or other fluid flows are established within circuitry 1006:
a) from pulmonary artery PA through inner lumen of cannula 1010 via pump P10a through outer lumen of cannula 1040 to left atrium LA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.

Flow a) is closed via right and left pulmonary veins PV, tissue of the lungs and right and left pulmonary arteries, and pulmonary artery PA. Flow b) is closed via arteries of the body, for instance common femoral artery CFA, tissues of the body and the veins of the body, for instance common femoral vein CFV.

No extra care has to be taken because both cannulas 1010 and 1040 are inserted into veins in which there is comparably low blood pressure compared to blood pressure in arteries.

The arrangement shown in FIG. 10 may be used for patients with lung problems. Mobility of the patient is possible because no cannulas in femoral veins or arteries are used. The arrangement shown in FIG. 10 may be named pIVLP (percutaneous in vivo lung perfusion) retrograde.

In other embodiments it is possible to insert cannula 1010 through internal jugular vein IJV/left subclavian vein to pulmonary artery PA as described above and cannula 1040 through right internal jugular vein IJV/right subclavian vein to ascending aorta aAO.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during retrograde lung perfusion. An optional cage arrangement may be arranged around inlet portion 1098.

During treatment of the lung L it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively, other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

Furthermore, FIG. 10 illustrates an extra corporeal antegrade lung perfusion circular blood flow circuitry 1006 comprising two dual lumen cannulas 1010 and 1040, two pumps P10a, P10b and an oxygenator device OXY10. The proposed antegrade lung perfusion uses the arrangement 1006 of cannulas 1010 and 1040 as described above for retrograde lung perfusion. Reference is made to the description above in order to avoid unnecessary repetition. However, only the differences will be described. The main difference is that the direction of fluid flow in pump P10a is in the opposite direction now, see arrows 1094 and 1095. The direction of the blood flow or other fluid flow within the veins and arteries of the lung L is now antegrade.

Arrow 1096 shows that fluid is expelled from the distal end 1012 of cannula 1010 into pulmonary artery PA. Holes 1015 are outlet holes for antegrade lung perfusion and optional tip 1014 is an optional outlet tip antegrade lung perfusion. However, alternatively, a single end-hole may be used. Membrane 1014 directs fluid flow into pulmonary artery PA completely or almost completely. Furthermore, membrane 1019 may have a valve function allowing blood/fluid flow from right ventricle into pulmonary artery but not in the inverse direction.

Arrow 1097 shows that blood or other fluid that comes out of pulmonary veins PV is extracted by suction into outer lumen of cannula 1040. Holes 1085 are inlet holes for antegrade lung perfusion and portion 1084 is an inlet portion for antegrade lung perfusion. Membrane 1089 directs fluid flow from pulmonary veins PV completely or almost completely into outer lumen of cannula 1040.

In summary, the following flows are established for antegrade lung perfusions within circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.

Flow a) is closed via pulmonary artery PA, right pulmonary artery rPA/left pulmonary artery lPA, tissue of the lung L and right/left pulmonary veins PV. Flow b) is closed via arteries of the body, for instance common femoral artery CFA, tissues of the body and the veins of the body, for instance common femoral vein CFV.

Even for antegrade lung perfusion, no extra care has to be taken because both cannulas 1010 and 1040 are inserted into veins in which blood pressure is comparably low compared to blood pressure in arteries.

The arrangement shown in FIG. 10 may be used for patients with lung problems. Mobility of the patient is possible because no cannulas in femoral veins or arteries are used. The arrangement for antegrade lung perfusion shown in FIG. 10 may be named pIVLP (percutaneous in vivo lung perfusion) antegrade.

Also for antegrade lung perfusion, it is possible to insert cannula 1010 through left internal jugular vein IJV/left subclavian vein to pulmonary artery PA as described above and cannula 1040 through right internal jugular vein IJV/right subclavian vein to ascending aorta aAO.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during antegrade lung perfusion.

During treatment of the lung L it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively, other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

Furthermore, it is possible to switch fluid flow direction between antegrade and retrograde, starting with antegrade fluid flow in lung L vessels or with retrograde fluid flow whichever is appropriate. Switching may be repeated during one treatment as often as necessary. Switching may ease the removal of at least one thrombus, especially of a blood thrombus.

Moreover, FIG. 10 illustrates an extra corporeal lobe dedicated antegrade lung perfusion circular blood flow circuitry 1006 comprising two dual lumen cannulas 1010 and 1040, two pumps P10a, P10b and an oxygenator device OXY10. The proposed lobe dedicated antegrade lung perfusion uses arrangement 1006 of cannulas 1010 and 1040 as described above for retrograde lung perfusion. Reference is made to the description above in order to avoid unnecessary repetition. However, only the differences will be described. One main difference is that the direction of blood/fluid flow in pump P10a is opposite to the direction mentioned above, see arrows 1094 and 1095. The other direction of fluid flow results in a change of the direction of the blood flow or other fluid flow within the veins and arteries of the lung.

A further difference is that cannula 1010 would have a longer portion between inlet portion 1090 and distal end 1012 enabling an arrangement of a cage arrangement 1016a within left pulmonary artery lPA as shown in FIG. 10. Cage arrangement 1016a is adapted to the diameter of left pulmonary artery lPA, i.e. it would be smaller than cage arrangement 1016. The other features of cage arrangement 1016a would be similar to the corresponding features of cage arrangement 1016 and would have an appropriate reduction in size, i.e. cage wires, membrane, optional outlet tip etc.

However, it is also possible to use an inflatable balloon instead of cage arrangement 1016a, see description of FIGS. 26 and 27 below.

The membrane of cage arrangement 1016a directs fluid flow that is expelled through inner lumen of cannula 1040 completely or almost completely into left pulmonary artery lPA. Furthermore, this membrane may have a valve function allowing blood flow from pulmonary artery PA also into left pulmonary artery lPA but not in the inverse direction.

Arrow 1097 shows that blood or other fluid that comes out of pulmonary veins PV is extracted by suction into outer lumen of cannula 1040 that is unchanged. Holes 1085 are inlet holes for antegrade lung perfusion and portion 1084 is an inlet portion for antegrade lung perfusion. Membrane 1089 directs fluid flow from pulmonary veins PV completely or almost completely into outer lumen of cannula 1040. Right pulmonary veins rPV will expel the fluid that is injected by cage arrangement 1016 and left pulmonary veins lPV will expel normal blood flow.

In summary, the following flows are established for dedicated antegrade lung perfusions within modified circuitry 1006:
a) from left atrium LA through outer lumen of cannula 1040 via pump P10a through inner lumen of cannula 1010 to pulmonary artery PA, i.e. lung perfusion, and
b) from right atrium RA and/or right ventricle RV through outer lumen of cannula 1010 via pump P10b and OXY10 through inner lumen of cannula 1040 to ascending aorta aAO, i.e. external enrichment of blood with oxygen.
c) normal blood flow from right ventricle RV through pulmonary artery PA, through right pulmonary artery rPA, tissue of lung L back via right pulmonary vein rPV into left atrium LA.

Flow a) is closed via left pulmonary artery lPA, tissue of left lung lobe of lung L and left pulmonary veins lPV. Flow b) is closed via arteries of the body, for instance common femoral artery CFA, tissues of the body and the veins of the body, for instance common femoral vein CFV.

Even for lobe dedicated antegrade lung perfusion, no extra care has to be taken because both cannulas 1010 and 1040 are inserted into veins in which there is comparably low blood pressure compared to blood pressure within arteries.

The modified arrangement shown in FIG. 10 may be used for patients with lung problems. Mobility of the patient is possible because no cannulas in femoral veins or arteries are used. The arrangement for dedicated lobe antegrade lung perfusion shown in FIG. 10 may be named pIVLP (percutaneous in vivo lung perfusion) antegrade lobe dedicated.

Also for dedicated lobe antegrade lung perfusion, it is possible to insert cannula 1010 through left internal jugular vein IJV/left subclavian vein to pulmonary artery PA as described above and cannula 1040 through right internal jugular vein IJV/right subclavian vein to ascending aorta aAO.

In another embodiment the right lobe of lung L may be flushed in the same way as described above for the left lobe of lung L. In this case, cage arrangement 1016a of cannula 1010 having a longer portion between inlet portion 1090 and distal end 1012 than shown in FIG. 10 would be arranged within right pulmonary artery rPA. Left pulmonary artery lPA would be filled with normal blood flow coming from right ventricle RV.

Furthermore, treatment of both lobes of lung L is possible is possible sequentially, e.g. treating the left lobe first and then the right lobe or vice versa. Several changes of the lobes that are treated are possible as well. The afterload that arises within heart H may be reduced in this way. Further positive effects may be possible as well. Detrimental effects may be limited to only one lobe. After a period of recreation, the other lobe may be treated. Moreover, there may be disease that require only the treatment of one lobe of lung L, for instance a thrombus in only one of the lobes of lung L.

An optional inlet portion 1098 may be arranged on a part of the outer lumen of cannula 1010 that is within right ventricle RV if cannula 1010 is put in place. Thus, it is possible to extract more blood from the right side of heart H using inlet portions 1090 and 1098 during dedicated lobe antegrade lung perfusion.

During dedicated treatment of only one lobe of the lung L it is possible that the patient inhales a medicament or treatment substance in order to promote the treatment by the substance or medicament that flows through the vessels of the lung L and through the tissue of the alveoli. The fluid flow within the part of circuitry 1006 which comprises pump P10a may comprise blood as a carrier substance. Alternatively, other carrier substances may be used, for instance based on saline and/or on water. The treatment substance may also be injected into the part of circuitry 1006 that comprises pump 10a. Furthermore, an adsorber/filter unit ADS and/or an oxygenator OXY and/or a carbon dioxide removal unit may be arranged within the part of circuitry 1006 that comprises pump 10a.

A dedicated retrograde treatment of the lobes of lung L seems feasible if further measures are taken, for instance usage of at least one split tip cannula, for instance within the left pulmonary veins lPV or within the right pulmonary veins rPV, preferably comprising at least two border elements, preferably expandable border elements, see FIGS. 26 and 27.

### D) RIGHT VENTRICLE ASSIST

FIG. 11 illustrates a right ventricle assist circuitry 1106 with one inlet stage or with multi inlet stages. Circuitry 1006 comprises one dual lumen cannula 1110 and one pump P11. Dual lumen cannula 1110 carries a cage arrangement 1146 near at least one outlet port that is arranged in pulmonary artery PA and at least one inlet portion 1190 that is arranged in right atrium RA. A second optional inlet portion 1198 may be arranged within right ventricle when cannula 1110 is arranged in place as shown in FIG. 11. It is also possible to use only inlet portion 1198 in right ventricle RV without having inlet portion 1190. Circuitry 1006 may be used for right ventricle assist.

Dual lumen cannula 1110 is inserted through left internal jugular vein IJV, superior vena cava SVC, right atrium RA, right ventricle RV, through pulmonary valve PVa into pulmonary artery PA. A guide wire (not shown) may be used to guide cannula 1110 to its final position. Alternatively, cannula 1010 may be inserted through the left subclavian vein and then along the same way as described above.

Almost the whole blood that comes out of inner lumen of cannula 1110 is injected into pulmonary artery PA, see arrow 1170, by using a membrane 1149 that is explained in more detail below. Other possibilities for insertion of a dual lumen cannula 1110 will be explained below, e.g. first insertion of outer lumen and then insertion of inner lumen.

Inlet portion 1190 may comprise a plurality of inlet holes that extend through the side wall of the outer lumen of cannula 1110. Blood is extracted by suction from right atrium RA into outer lumen of cannula 1110, preferably all blood or nearly all (for instance more than 90 percent of volume) blood that comes into right atrium RA, see arrow 1160. Optional inlet portion 1198 may comprise a plurality of inlet holes that extend through the side wall of the outer lumen of cannula 1110. Blood is extracted by suction from right ventricle RV into outer lumen of cannula 1110, preferably all blood or nearly all (for instance more than 90 percent of volume) blood that comes into left ventricle LV, see arrow 1199.

With reference further to FIG. 11, a tube 1120a is connected to a proximal end of outer lumen of cannula 1110 and to an inlet of pump P11. An outlet of pump P11 may be connected to a proximal end of inner lumen of cannula 1110 using a tube 1030. Pump P11 may be peristaltic pump or a centrifugal pump.

Tubes 1120, 1130 may be made of a flexible material or of a more rigid material. The circuitry 1106 may further include one or more blood filter units or units for dialysis of blood.

Cannula 1140 may comprise an optional outlet tip 1150. Outlet tip 1150 may comprise a plurality of outlet holes 1152 in its side wall and/or on its distal end. Additionally, cannula 1110 may have an optional cage arrangement 1146 on its distal end 1142.

Cage arrangement 1146 is one possible example. Other possible examples are described below with reference to FIGS. 16 to 20. Cage arrangement 1146 may comprise for instance between 6 to 12 flexible wires, beams or bars. There may be for instance 8 cage wires 1148 that span a sphere. The sphere and also the expelled blood prevent that the side wall of the pulmonary artery PA covers one of outlet holes 1152 of optional outlet tip 1150. Furthermore, cage arrangement 1146 fixes distal end 1142 of cannula 1140 within pulmonary artery PA.

Membrane 1149 covers only one half of cage arrangement 1146, e.g. a half that is between distal end 1142 of cannula 1140 and mid of cage wires 1148. Examples of membranes that may be used on cage arrangement 1148 are described below with reference to FIG. 16. Membrane 1149 directs blood into pulmonary artery PA and prevents that blood flows back into right ventricle RV. Membrane 1178 may have a valve function, e.g. if remaining blood comes from right ventricle RV it can pass between membrane 1149 and sidewalls of pulmonary artery PA.

The following blood flow that is established within circuitry 1106 is from right atrium RA and/or right ventricle RV through outer lumen of cannula 1110 via pump P11 back through inner lumen of cannula 1110 to pulmonary artery. This flow is closed via right and left pulmonary veins PV, arteries of body 100, for instance common femoral artery CFA, tissue of body 100 and veins of body 100, for instance common femoral vein CFV.

The arrangement shown in FIG. 11 may be used for patients without lung problems. Mobility of the patient is possible because no cannulas in femoral veins or arteries are used. The arrangement for shown in FIG. 11 may be named pRVAD (percutaneous right ventricle assist device) multi lumen.

It is possible to insert cannula 1110 through right internal jugular vein IJV/right subclavian vein to pulmonary artery PA as described above.

Other applications of the proposed cage arrangements and/or dual lumen cannulas than these shown in FIGS. 1 to 11 are possible as well. All cannulas shown may be used with or without cages.

The cannula systems CS1 to CS3 that are described with reference to FIGS. 12 to 14 are further examples for dual lumen cannula systems shown in FIGS. 2, 3, 10 and 11. FIG. 12 illustrates a cannula system CS1 having an inner cannula I1 and an outer cannula O1 that are arranged coaxially relative to each other with the inner cannula I1 arranged inside the outer cannula O1. Outer cannula O1 is named as a first cannula in the claims. Inner cannula I1 is named as a second cannula in the claims.

Outer cannula O1 is inserted into body 100 first, i.e. preferably before the inner cannula I1 will be inserted. Only after the insertion of the outer cannula O1 into body 100, preferably after the insertion of outer cannula O1 is completed, i.e. the outer cannula O1 has reached its destination position, inner cannula I1 is inserted into outer cannula O1 and then further beyond the distal end of outer cannula O1.

Both cannulas O1 and I1 are bendable up to a specific degree, i.e. they are bendable in radial directions. However, the diameter of cannulas O1 and I1 may not be variable in the sense that the area of the diameter cross section may be increased or decreased essentially.

Outer cannula O1 may have a circular or oval cross section along its entire length. A port P1a of outer cannula O1 may be arranged at a proximal P end of a sidewall of outer cannula O1. The proximal P end of outer cannula O1 may comprise a proximal surface, for instance a flat surface, that may have an opening OP1. Opening OP1 may be arranged on the longitudinal axis A of outer cannula O1.

Inner cannula I1 may also have a circular or oval cross section along its entire length. A port P1b of inner cannula I1 may be arranged at a proximal P end of inner cannula I1. Inner cannula I1 may be inserted through opening OP1 into outer cannula O1. Thereby, the inner cannula I1 may be arranged on the longitudinal axis A of outer cannula O1. At least one mounting portion MP1 or mounting elements may be arranged on an outer surface of inner cannula I1, e.g. protruding radially outward, and/or on an inner surface of outer cannula O1, e.g. protruding radially inward. Mounting portions MP1 may center inner cannula I1 within outer cannula O1.

A sealing element S1 may be used to seal cannula system CS1 proximally. Sealing element S1 may be arranged within opening OP1 or at another appropriate location. Sealing element S1 may be an O-ring in the simplest case. Alternatively, a multi-flap valve or a membrane may be used.

An optional fixation element FE1 may be arranged completely outside of outer cannula O1. Fixation element FE1 may have a first state in which axial movement M1 of inner cannula I1 relative to outer cannula O1 is possible or allowed and a second state that blocks such axial movement. Fixation element FE1 may operate automatically or semi-automatically or may be operated manually. Thus, fixation element FE1 may block axial movement if a predetermined length of inner cannula I1 is introduced into outer cannula O2. Alternatively, blocking may be performed manually at several positions of inner cannula I1 within outer cannula O1. It may be possible to bring fixation element FE1 back to the first state after it is in the blocking state.

Alternatively, fixation element FE1 may be arranged partly or completely within outer cannula O1. If it is completely within outer cannula O1 manual access to fixation element FE1 may be possible by operating elements. Alternatively, no manual access may be possible, i.e. fixation element FE1 may be operated in an automatic or semi-automatic mode depending for instance on the overlapping length of both cannulas I1 and O1.

FIG. 13 illustrates a cannula system CS2 having an inner (second) cannula I2 that is arranged loosely within an outer (first) cannula O2. Outer cannula O2 is named as a first cannula in the claims. Inner cannula I2 is named as a second cannula in the claims.

Outer cannula O2 is inserted into body 100 first, i.e. preferably before the inner cannula I2 will be inserted. Only after the insertion of the outer cannula O2 into body 100, preferably after the insertion of outer cannula O2 is completed, i.e. the outer cannula O2 has reached its destination position, inner cannula I2 is inserted into outer cannula O2 and then further beyond the distal end of outer cannula O2.

Both cannulas O2 and 12 are bendable up to a specific degree, i.e. they are bendable in radial directions. However, the diameter of cannulas O2 and I2 may not be variable in the sense that the area of the diameter cross section may be increased or decreased essentially.

Outer cannula O2 may have a circular or oval cross section along its entire length. A port P2a of outer cannula O2 may be arranged at a proximal P end of outer cannula O2 that may be arranged on the longitudinal axis of outer cannula O2. The sidewall of outer cannula O2 may have an opening OP2 at its proximal P end. Opening OP2 may face laterally and or transversally relative to longitudinal axis A of outer cannula O2.

Inner cannula I2 may also have a circular or oval cross section along its entire length. A port P2b of inner cannula I2 may be arranged at a proximal P end of inner cannula I1. Inner cannula I2 may be inserted through opening OP2 into outer cannula O2. Thereby, the inner cannula I1 may be arranged loosely radially to longitudinal axis A of outer cannula O1. A mounting portion may not be necessary.

A sealing element S2 may be used to seal cannula system CS2 proximally. Sealing element S2 may be arranged within opening OP2 or at another appropriate location. Sealing element S2 may be an O-ring in the simplest case. Alternatively, a multi-flap valve or a membrane may be used.

An optional fixation element FE2 may be arranged completely outside of outer cannula O2. Fixation element FE2 may have a first state in which an axial movement M2 of inner cannula I2 relative to outer cannula O2 is possible or allowed and a second state that blocks such axial movement. Fixation element FE2 may operate automatically or semi-automatically or may be operated manually. Thus fixation element FE2 may block axial movement if a predetermined length of inner cannula I2 is introduced or inserted into outer cannula O2. Alternatively, blocking may be performed manually at several positions of inner cannula I2 within outer cannula O2. It may be possible to bring fixation element FE2 back to the first state after it is in the blocking state.

Alternatively, fixation element FE2 may be arranged partly or completely within outer cannula O2. If it is completely within outer cannula O2 manual access to fixation element FE2 may be possible by operating elements. Alternatively, no manual access may be possible, i.e. fixation element FE2 may be operated in an automatic or semi-automatic mode depending for instance on the overlapping length of both cannulas I2 and O2.

FIG. 14 illustrates a cross section of another cannula system CS3 that comprises an outer cannula O3 and an inner cannula I3. Outer cannula O3 is named as a first cannula in the claims. Inner cannula I3 is named as a second cannula in the claims.

Outer cannula O3 has a circular inner cross section, preferably along its whole length. Alternatively, outer cannula O3 may have an oval or elliptic inner cross section, preferably along its whole length.

Inner cannula I3 has an outer cross section that is complementary to the inner cross section of outer cannula O3 and that leaves a lumen (first lumen in the claims) for the transport a fluid through outer cannula O3. If outer cannula O3 has an oval inner cross section, the outer cross section of inner cannula may be also oval or elliptic minus a part that is used for fluid transport in outer cannula O3.

The fluid may be blood or may comprise blood, for instance blood enhanced with a medicament or drug. Alternatively, other fluids than blood may be used.

Inner cannula I3 may have a flat outer surface that is arranged for instance along the longitudinal axis A of outer cannula O3. Alternatively, this flat surface of inner cannula I3 may be arranged on a side of the longitudinal axis A of outer cannula O3 on which the first lumen of the outer cannula O3 for fluid transport is located, see line L3. In a further alternative, the flat surface of inner cannula I3 may be arranged on a side of the longitudinal axis A of outer cannula O3 that is opposite to the side that comprises the main part of the first lumen of the outer cannula O3 for fluid transport, see line L4.

No mounting elements are necessary in cannula system CS3. However, it is possible to use mounting elements that position or fix the inner cannula I3 radially relative to outer cannula O3. Positioning would be easier than in cannula system CS1 because the complementary shapes of inner cannula I3 and outer cannula O3 may be used to enhance a specific positioning of inner cannula I3 within outer cannula O3.

FIG. 15 illustrates an embodiment of a dual lumen cannula system 1500 comprising at least one pre bended outer cannula 1508 and an inner cannula that is not shown. Cannula system 1500 is adapted for a stepwise insertion of the cannula 1508 and of the other cannula, i.e. first outer cannula 1508 and then inner cannula. Dual lumen system 1500 may comprise:
a locking mechanism 1502 that may lock an introducer that is used for introducing outer cannula 1508. FIG. 21 shows an example in which an introducer is inserted into a cannula system. The introducer may be locked by force fitting or by another appropriate mechanism.
an adapter portion 1504 that may be used to connect locking mechanism removably to cannula system 1500,
a handle portion 1506 that may also be connected removably to cannula system 1500 and that is used to ease introducing of outer cannula 1508 or inner cannula into body 100 of a patient. Handle portion 1506 may be compressible by a medical clamp or forceps.

There may be a pre-bended kink K or bend that is between a long straight portion of cannula 1508 of system 1500 and a shorter straight portion. Cannula 1508 may have a straight insertable length L10 up to kink K and a short straight portion of cannula system 1500 having an insertable length L20 between kink K and the distal end. Kink K may also be positioned on other positions than the position shown in FIG. 15, e.g. more distally D or more proximally P. Cannula system 1500, especially cannula 1508, is or may be flexible, i.e. it is possible to bend each portion and of course to bring the whole cannula system 1500 in a straight shape. However, without external forces, kink K will bring cannula 1508 of system 1500 in the shape that is shown in FIG. 15 again. The inner cannula of cannula system 1500 may not have a kink K but may be straight. Alternatively, also the inner cannula of cannula system 1500 may have a kink. The cannula comprising or having the bend may be used for endovascular jugular insertion into the left atrium or into the pulmonary artery.

Although, cannula 1508 is shown having no diameter variable arrangement DVA or cage arrangement on the tip there may be such a diameter variable arrangement DVA1. Cannula 1508 may comprise only one end-hole that may be closed by a closure element that allows passage of the inner cannula but not of blood into cannula 1508 through the end-hole or vice versa. The lateral or side holes of cannula 1508 may be placed within the right atrium RA of the heart H whereas the cage arrangement may be placed within the left atrium LA of the heart H. Insertion of the inner cannula may further be promoted if at least one wire of diameter variable arrangement DVA1 is omitted.

In an alternative embodiment a cage arrangement or diameter variable arrangement DVA2 is used and the distal tip with lateral holes is omitted. There may be a membrane connected to diameter variable arrangement DVA2 that has an opening which faces laterally, see cage arrangement 1086 in FIG. 10 and in the variants of FIG. 10. Cannula 1508 may have only one end-hole in this case, preferably an end-hole that is not closed by a valve and/or membrane. Introduction of the inner cannula of cannula system 1500 may be easier compared to the case in which a distal tip having lateral holes is include within diameter variable arrangement DVA2. However, in a further alternative, a distal tip with lateral holes and/or with an end hole may be used. Insertion of the inner cannula may further be promoted if at least one wire of diameter variable arrangement DVA2 is omitted.

Cannula 1508 may be used as a delivery cannula or as a drainage cannula.

Cannula system 1500 may be used for jugular access to heart H or for other purposes. Length L10 refers to the length from the proximal end of handle portion 1506 to the pre-bended kink K, i.e. the length of the longer straight portion of cannula 1508. An example for length L10 is 300 mm. Other values for length L10 are also possible.

Length L20 is the length of the pre-bended distal portion, i.e. measured from the pre-bended kink K to the distal end of cannula 1508, and without the length of a diameter variable arrangement if present. An example for length L20 may be for instance 70 mm (millimeter). Other values for length L20 are possible as well. Preferred values for length L20 are within the range of 3 cm to 7 cm.

An angle W1 between the two straight portions of cannula system 1500 at kink K may have a value of 130 degrees. However, a value within the range of 70 degrees to 145 degrees is also possible.

Cannula system 1500 may be used for left or right jugular or for left or right subclavian access to heart H or for other purposes. Longer cannulas are necessary for left side access and or for femoral access to the heart H. Modifications may be made with regard to length L10 and or length L20. Furthermore, the kink K may be at another position and angle W1 may have another value. It may also be useful to have a second pre-bended kink.

For cannula 1508 of cannula system 1500 the following table may be valid:

| size of cannula in F/ | | | |
|---|---|---|---|
| overall length in cm | SL1 in mm | SL2 in mm | L10 + L20 in cm |
| 21 F/32 | 7.0 | 2.3 | 32 |
| 21 F/42 | 7.0 | 2.3 | 42 |
| 21 F/62 | 7.0 | 2.3 | 62 |
| 21 F/72 | 7.0 | 2.3 | 72 |
| 31 F/42 | 10.3 | 2.3 | 42 |

There may be further intermediate sizes of cannulas having for instance an outer diameter of 23 F (French), 25 F, 27 F and 29 F combined with an overall length L10 plus L20 of for instance 32 cm, 42 cm or 62 cm. The overall length L10 plus L20 is the implantable length of cannula 1408.

SL1 is the outer diameter of outer or first cannula 1508 in French. SL2 is the diameter of lateral holes in the distal tip. The numbers given in the table or given above may vary within a range of minus 10 percent to plus 10 percent. Other sizes of the cannula system 1500 are possible as well.

The tip of inner cannula of cannula system that is shown in FIG. 15 may be optional. Furthermore, a cage arrangement (diameter variable arrangement) may be mounted on distal end of inner cannula. Additionally or alternatively, a cage arrangement (diameter variable arrangement) may be mounted on distal end of outer cannula 1508. None, one or both cage arrangements (diameter variable arrangement) may be covered by a respective membrane. The membrane of outer cannula 1508 may have an opening that faces distally or laterally or proximally. The membrane of the inner cannula may have an opening that faces distally, see for instance cannula 1040 in FIG. 10.

FIGS. 16 to 21 show embodiments of cage arrangements that may be used in all of the embodiments shown in FIGS. 1 to 11. FIG. 16 illustrates a cage arrangement 1600 comprising a membrane 1650 having an opening 1652 that faces distally. The shape of cage arrangement 1600 in its expanded state may be similar to a sphere or to a ball. Alternatively, an ellipsoid or another shape may be used. Cage arrangement 1600 is mounted to a cannula 1602 that may be an outer cannula or an inner cannula of a cannula system, for instance of one of cannula systems CS1 to CS3.

Cannula 1602 may comprise an optional cannula tip 1604 having apertures 1606, 1608 arranged in the pattern that is shown. However, other arrangements of apertures 1606, 1606 may be used, especially comprising a different number of apertures 1606, 1606. If cannula tip 1604 is not used there may be a single end-hole at the distal tip of cannula 1602. Cannula 1602 may not extend or may only extend by less than 10 mm into cage arrangement 1600.

If cage arrangement 1600 is viewed from above, it comprises in a counter clock wise direction eight cage wires 1610, 1612, etc. to 1624. More or less cage wires 1610 to 1624 may also be used. Cage wire 1624 is at the rear side of cage arrangement 1600. Cage wires 1610 to 1624 have, at a given axial position, same distances, especially same angularly distances, to the neighboring cage wires 1610 to 1624. One of the cage wires 1610 to 1624 or some of the cage wires 1610 to 1624 may be omitted, for instance to allow the insertion of further cannulas and/or cage arrangements through cage arrangement 1600.

Cage arrangement 1600 may have the following portions with increasing distance from mounting portion 1630:
a mounting portion 1630 at which the cage wires 1610 to 1624 are wound around distal end of cannula 1602, for instance at least three quarters of the circumference. Mounting portion 1630 may alternatively comprise an additional mounting element on which cage wires 1610 to 1624 are mounted, for instance a mounting sleeve or a jacket. In both cases a circumferential notch in cannula 1602 may be used to prevent axial movement of cage arrangement 1600 relative to cannula 1602. Additional or alternative mounting techniques may be used, i.e. welding, soldering, glue etc.
a proximal portion 1631 in which neighboring cage wires have increasing distances with regard to each other and with increasing distance to mounting portion 1630,
a comparably short optional transition portion 1632 in which cage wires 1610 to 1624 are arranged essentially parallel relative to each other and/or the longitudinal axis of cannula 1602 as well as to the longitudinal axis of cage arrangement 1600,
a distal portion 1633 in which neighboring cage wires have decreasing distances with regard to each other and with increasing distance to mounting portion 1630, and
a cage tip portion 1635 in which the cage wires 1610 to 1624 are connected together, for instance by a plastic cap. Within tip portion 1635 cage wires 1610 to 1624 may be twisted or be arranged parallel with regard to each other.

All cage wires 1610 to 1624 may be pre-bended in the same way and/or may have the same shape memorized within the shape memory of the material of the cage wires 1610 to 1624. An example is given for cage wire 1612 that is arranged mainly within a plane that is equal to the plane of the sheet that shows FIG. 16. Cage wire 1612 comprises portions that correspond to portions 1630 to 1635 of cage arrangement 1600:
a mounting portion 1640 that comprises:
   an optional circumferential portion 1638 in which the wire is shaped circular, for instance along at least three quarters of the circumference of a circle, and
   an optional straight portion 1639 that may be arranged parallel to the longitudinal axis of cage arrangement 1600 and that may serve as a reference axis in the following—alternatively the longitudinal axis of cannula 1602 may be uses as a reference axis,
a proximal portion 1641 in which wire 1612 has an increasing radial distance to the reference axis with increasing distance to mounting portion 1640,
an optional transition portion 1642 in which wire 1612 has a constant radial distance to the reference axis with increasing distance to mounting portion 1640,
a distal portion 1643 in which wire 1612 has a decreasing radial distance to the reference axis with increasing distance to mounting portion 1640, and
a cage tip portion 1645 that may be covered by plastic cap and/or in which the wire 1612 is parallel to the reference axis or is spirally and/or helically wounded.

Membrane 1650 extends circumferential from proximal P end almost up to distal D end of cage arrangement 1600, i.e. portions 1631 and 1632 are covered completely and portion 1633 is covered at more than half of its axial length. An opening 1652 faces distally to distal cage tip 1654 relative to longitudinal axis of cannula 1602 or of cage arrangement 1600.

Membrane 1650 may cover only or at least the lower half or only or at least the upper half of cage arrangement 1600, see line. In the latter case the opening of membrane 1650 would be facing proximally. Membrane 1650 may cover also only the lower quarter or the lower three quarters of cage arrangement 1600. Reference may be made thereby to the axial length of cage arrangement 1600. Further, membrane 1650 may cover also only the upper quarter or the upper three quarters of cage arrangement 1600. However, cage arrangement 1600 may also be used without membrane 1600.

A separate spirally wounded wire that forms a coil may be used to form a mounting portion that is similar to mounting portion 1630. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1638. The straight portions, for instance 1639, may be connected to the coil with the same axial position or with the same axial offset between angularly neighboring/adjacent wires, i.e. comparably to the arrangement that is shown in FIG. 16.

A sleeve may be used to form a mounting portion. The wires may have or may not have the circumferential portions, for instance 1638. The straight portions, for instance 1639, may be connected to the sleeve with the same axial position or with the same axial offset between angularly neighboring/adjacent wires, i.e. comparably to the arrangement that is shown in FIG. 16. The sleeve may be an inner sleeve relative to the wires or an outer sleeve. Furthermore, it is possible to use an inner sleeve and an outer sleeve with portions of the wires arranged therein between, especially straight portions that are arranged in parallel or oblique to a longitudinal axis of the sleeve and/or of the cannula.

Other possibilities of the connection of the cage arrangement to the cannula may be used as well.

FIG. 17 illustrates a cage arrangement 1700 comprising a membrane 1750 having an opening that faces laterally. However, cage arrangement 1700 corresponds to cage arrangement 1600 except of the placement of membrane 1750. In order to avoid repetition reference is made to the description of FIG. 16 above. There are the following corresponding parts:
cage arrangement 1600, 1700,
cannula 1602, 1702,
optional cannula tip 1702, 1704,
apertures 1606, 1608, 1706, 1708,
cage wires 1610 to 1624, 1710 to 1724,
cage portions 1630 to 1635 are also valid for cage arrangement 1700,
circumferential portion 1638, 1738,
straight portions 1639, 1739,
cage wire portions 1640 to 1645 are also valid for cage arrangement 1700, and
cage tip 1654, 1754.

Features that are mentioned above for parts 1600 to 1645 apply also to the corresponding parts 1700 to 1739 and to the corresponding parts that are not indicated by reference signs in FIG. 17.

Membrane 1750 covers slightly more than half of cage arrangement 1700 and has an opening 1752 that faces laterally or transversally relative to the longitudinal axis of cannula 1702 or of cage arrangement 1700. Thus, in the expanded state of cage arrangement 1700, membrane 1750 is arranged between cage wires 1718, 1720; 1720, 1722; 1722, 1724; 1724, 1710 and 1710, 1712. Membrane 1750 may extend through all main portions of cage arrangement 1700 between these cage wires, i.e. proximal portion, optional transition portion and distal portion, see corresponding portions 1631 to 1633 in FIG. 16.

Other arrangements of membrane 1750 are possible as well each having an opening that faces laterally:
less than 90 degrees in circumferential direction, preferably more than 10 degrees or more than 45 degrees,
90 degrees or more than 90 degrees of coverage in circumferential direction, but preferably less than 110 degrees, less than 135 degrees or less than 180 degrees,
180 degrees of coverage, i.e. membrane 1750 is only arranged between cage wires 1720 to 1724 and further between cage wire 1724 and 1710 as well as between cage wire 1710 and 1712, alternatively there may be at least 180 degrees of coverage, or
270 degrees of coverage, i.e. membrane 1750 is arranged additionally between cage wires 1716 and 1718, alternatively there may be at least 270 degrees of coverage.

Other angles of coverage for membrane 1750 may be easily realized if more or less than eight cage wires 1710 to 1724 are used in cage arrangement 1700. However, cage arrangement 1700 may also be used without a membrane. Combinations of lateral and distal/proximal facing openings are also possible.

A separate spirally wounded wire that forms a coil may be used to form a mounting portion that is similar to mounting portion 1730. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1738. The straight portions, for instance 1739, may be connected to the coil with the same axial position or with the same axial offset between angularly neighboring/adjacent wires, i.e. comparably to the arrangement that is shown in FIG. 17.

A sleeve may be used to form a mounting portion. The wires may have or may not have the circumferential portions, for instance 1738. The straight portions, for instance 1739, may be connected to the sleeve with the same axial position or with the same axial offset between angularly neighboring/adjacent wires, i.e. comparably to the arrangement that is shown in FIG. 17. The sleeve may be an inner sleeve relative to the wires or an outer sleeve. Furthermore, it is possible to use an inner sleeve and an outer sleeve with portions of the wires arranged therein between, especially straight portions that may be arranged in parallel or oblique to a longitudinal axis of the sleeve and/or of the cannula.

Other possibilities of the connection of the cage arrangement to the cannula may be used as well.

FIG. 18 illustrates a cage arrangement 1800 comprising a portion that is bended backwards, i.e. backwards bended portion 1834. The shape of cage arrangement 1800 in its expanded state may be similar to a sphere or to a ball. Alternatively, an ellipsoid or another shape may be used. Cage arrangement 1800 is mounted to a cannula 1802 that may be an outer cannula or an inner cannula of a cannula system, for instance of one of cannula systems CS1 to CS3.

Cannula 1802 may comprise an optional cannula tip 1804 having apertures 1806, 1808 arranged in the pattern that is shown. However, other arrangement of apertures 1806, 1806 may be used, especially comprising a different number of apertures 1806, 1806. If cannula tip 1804 is not used, there may be a single end-hole at the distal tip of cannula 1802. Cannula 1802 may not extend or may only extend by less than 10 mm into cage arrangement 1800.

If cage arrangement 1800 is viewed from above, it comprises in a counter clock wise direction eight cage wires 1810, 1812, etc. to 1824. More or less cage wires 1810 to 1824 may also be used. Cage wire 1810 to 1824 is at the rear side of cage arrangement 1600. Cage wires 1810 to 1824 have at a given axial position same distances to the neighboring cage wires 1810 to 1824. One of the cage wires 1810 to 1824 or some of the cage wires 1810 to 1824 may be omitted, for instance to allow the insertion of further cannulas and/or cage arrangements through cage arrangement 1800.

Cage arrangement 1800 may have the following portions with increasing distance from mounting portion 1830:
a mounting portion 1830 at which the cage wires 1810 to 1824 are wound around distal end of cannula 1802, for instance at least three quarters of the circumference. Mounting portion 1830 may alternatively comprise an additional mounting element on which cage wires 1810 to 1824 are mounted, for instance a mounting sleeve or a jacket. In both cases a circumferential notch in cannula 1802 may be used to prevent axial movement of cage arrangement 1800 relative to cannula 1802. Additional or alternative mounting techniques may be used, i.e. welding, soldering, glue etc.
a proximal portion 1831 in which neighboring cage wires have increasing distances with regard to each other and with increasing distance to mounting portion 1830,
a comparably short optional transition portion 1832 in which cage wires 1810 to 1824 are arranged essentially parallel relative to each other and/or the longitudinal axis of cannula 1802 as well as to the longitudinal axis of cage arrangement 1800, and
a distal portion 1833 in which neighboring cage wires have decreasing distances with increasing distance to mounting portion 1830.

There may be a short optional radial portion that forms a plane for contact with a wall of a vessel or a chamber of the heart. The radial length of this radial portion may be in the range of 3 mm to 10 mm (millimeters). In the expanded state, wire portions within the radial portion extend only radially but not axially, i.e. the wire portions have the same axial position and extend to the extended longitudinal axis of cannula 1802.

Furthermore, cage arrangement 1800 may comprise following the distal portion 1833:
a backwards bended portion 1834 in which cage wires 1810 to 1824 change direction and in which neighboring cage wires 1810 to 1824 have decreasing distances with regard to each other and with decreasing distance to mounting portion 1830, and
a cage tip portion 1835 in which the cage wires 1810 to 1824 are connected together, for instance by a plastic cap. Within tip portion 1835 cage wires 1810 to 1824 may be twisted or be arranged parallel with regard to each other.

All cage wires 1810 to 1824 may be pre-bended in the same way and/or may have the same shape memorized within the shape memory of the material of the cage wires 1810 to 1824. An example is given for cage wire 1812 that is arranged mainly within a plane that is equal to the plane of the sheet that shows FIG. 18. Cage wire 1812 comprises portions that correspond to portions 1830 to 1835 of cage arrangement 1800:
a mounting portion 1840 that comprises:
   an optional circumferential portion 1838 in which the wire is shaped circular, for instance along at least three quarters of the circumference of a circle, and
   an optional straight portion 1839 that may be arranged parallel to the longitudinal axis of cage arrangement 1800 and that may serve as a reference axis in the following—alternatively the longitudinal axis of cannula 1802 may be uses as a reference axis,
a proximal portion 1841 in which wire 1812 has an increasing radial distance to the reference axis with increasing distance to mounting portion 1840,
an optional transition portion 1842 in which wire 1812 has a constant radial distance to the reference axis with increasing distance to mounting portion 1840, and
a distal portion 1843 in which wire 1812 has a decreasing radial distance to the reference axis with increasing distance to mounting portion 1840.

There may be the optional radial portion that is mentioned above. The optional radial portion may be arranged between the distal portion 1843 and the backwardly bended wire portion 1844.

Furthermore, cage wire 1812 may comprise following the distal portion 1843:
a backwardly bended wire portion 1844, and
a cage tip portion 1845 that may be covered by plastic cap and/or in which the wire 1812 is parallel to the reference axis or is spirally and/or helically wounded.

In another embodiment cage arrangement 1800 may be covered at least partially by a membrane. The membrane may have an opening that faces distally or proximally, see description of FIG. 16, for instance coverage of lower three quarters. Alternatively, the membrane may have an opening that faces laterally, see description of FIG. 17, for instance coverage of at least half of the circumference or of three quarter of circumference. Combinations of lateral and distal/proximal facing openings are also possible.

Other shapes of cage arrangements with a backward bended portion are also possible, see for instance shapes similar to the shapes that are shown in FIGS. 19 and 20, e.g. cone shape or cylinder shape.

A separate spirally wounded wire that forms a coil may be used to form a mounting portion that is similar to mounting portion 1830. The number of windings within the coil may be in the range of 3 windings to 15 windings and/or in the range of 3 windings to 10 windings. There may be no space between adjacent or neighboring windings. Alternatively, there may be a small space between adjacent windings. The wires may have or may not have the circumferential portions, for instance 1838. The straight portions, for instance 1839, may be connected to the coil with the same axial position or with the same axial offset between angularly neighboring/adjacent wires, i.e. comparably to the arrangement that is shown in FIG. 18.

A sleeve may be used to form a mounting portion. The wires may have or may not have the circumferential portions, for instance 1838. The straight portions, for instance 1839, may be connected to the sleeve with the same axial position or with the same axial offset between angularly neighboring/adjacent wires, i.e. comparably to the arrangement that is shown in FIG. 18. The sleeve may be an inner sleeve relative to the wires or an outer sleeve. Furthermore, it is possible to use an inner sleeve and an outer sleeve with portions of the wires arranged therein between, especially straight portions that are arranged in parallel or oblique to a longitudinal axis of the sleeve and/or of the cannula.

Other possibilities of the connection of the cage arrangement to the cannula may be used as well.

FIG. 19 illustrates cannulas 1908, 1910 of a cannula system 1900. Cannula 1908 is an outer cannula 1908 of cannula system 1900. Cannula 1910 is an inner cannula 1910 of cannula system 1900. The length and/or outer diameters of cannulas 1908 and 1910 may be different, i.e. inner cannula 1910 may be longer and thinner than outer cannula 1908. Disregarding these differences, both cannulas 1908 and 1910 may correspond to the picture that is shown in FIG. 19. One of the cannulas 1908, 1910 or both cannulas 1908, 1910 may comprise a cage arrangement 1912.

Cannula system 1900 may be adapted for a stepwise insertion of the cannulas 1908 and 1910, i.e. first outer cannula 1908 and then inner cannula 1910. Dual lumen cannula system 1900 may comprise, preferably as separate systems for each cannula 1908 and 1910 or only one system for both cannulas 1908 and 1910:
a locking mechanism 1902 that may lock an introducer that is used for introducing outer cannula 1908 or inner cannula 1910, especially after outer cannula 1908 has been introduced. FIG. 21 shows an example in which an introducer or introducer member is inserted into a cannula system. The introducer may be locked by force fitting or by another appropriate mechanism.
an adapter portion 1904 that may be used to connect locking mechanism 1902 removably to cannula system 1900, and
a handle portion 1906 that may also be connected removably to cannula system 1900 and that is used to ease introducing of outer cannula 1908 or inner cannula 1910 into body 100 of a patient.

Inner cannula 1908 and/or outer cannula 1910 may comprise a cage arrangement 1912 having wires that are essentially arranged in parallel with regard to each other in the main portion of the cage arrangement 1912, e.g. along the entire axial length of cage arrangement 1912 or along at least 90 percent of this length. Thus cage arrangement 1912 has the shape of a cylinder.

Cage arrangement 1912 of inner cannula 1908 and/or outer cannula 1910 may have or may comprise a membrane, for instance a membrane that has an opening facing distally or laterally, see for instance FIG. 16 and FIG. 17 and respective corresponding descriptions.

Cannula 1908 and/or cannula 1910 may be pre-bended as described above for cannula system 1900. The tip of cannula 1908 and/or 1909 may be optional, i.e. there may be only one opening within cage arrangement 1912, for instance at its proximal end.

The cage arrangement 1912 may be used also for a single lumen cannula, preferably with or without a membrane.

FIG. 20 illustrates cannulas 2008, 2010 of a cannula system 2000 comprising a cage arrangement 2012 having a cone like shape. Cannula 1908 is an outer cannula 1908 of cannula system 1900. Cannula 2010 is an inner cannula 2010 of cannula system 2000. The length and/or outer diameters of cannulas 1908 and 1910 may be different, i.e. inner cannula 1910 may be longer and thinner than outer cannula 1908. Disregarding these differences, both cannulas 1908 and 1910 may correspond to the picture that is shown in FIG. 20. One of the cannulas 2008, 2010 or both cannulas 2008, 2010 may comprise a cage arrangement 2012.

Cannula system 2000 may be adapted or is adapted for a stepwise insertion of cannulas 2008 and 2010, i.e. first outer cannula 2008 and then inner cannula 2010. Dual lumen cannula system 2000 may comprise, preferably as separate systems for each cannula 2008 and 2010 or only one system for both cannulas 2008 and 2010:
a locking mechanism 2002 that may lock an introducer or introducer member that is used for introducing outer cannula 2008 or inner cannula 2010, especially after outer cannula 2008 has been introduced. FIG. 21 shows an example in which an introducer is inserted into a cannula system 2000. The introducer may be locked by force fitting or by another appropriate mechanism.
an adapter portion 2004 that may be used to connect locking mechanism 2002 removably to cannula system 2000, and
a handle portion 2006 that may also be connected removably to cannula system 2000 and that is used to ease introducing of outer cannula 2008 or inner cannula 2010 into body 100 of a patient.

Inner cannula 2008 and/or outer cannula 2010 may comprise a cage arrangement 2012 having wires that extend in the proximal portion of cage arrangement 2012 essentially radially outward. Within an optional short transition portion of cage arrangement 2012 the wires are parallel to each other and/or to the longitudinal axis of cannula 2008, 2010. Within a distal portion of cage arrangement 2012 the wires are arranged on a surface that would define the inclined surface of a cone. This distal portion may extend along almost the entire axial length of cage arrangement 2012 or along at least 90 percent of this length. Thus, it may be said that cage arrangement 2012 has the shape of a cone. Within the distal portion of cage arrangement 2012 the distances between neighboring wires are decreasing with increasing distance to a mounting portion of cage arrangement 2012.

Cage arrangement 2012 of inner cannula 2008 and/or outer cannula 2010 may have or may comprise a membrane, for instance a membrane that has an opening facing distally or laterally, see for instance FIG. 16 and FIG. 17 and respective corresponding descriptions.

Cannula 2008 and/or cannula 2010 may be pre-bended as described above for cannula system 2000. The tip of cannula 2008 and/or 2009 may be optional, i.e. there may be only one opening within cage arrangement 2012, for instance at its proximal end.

The cage arrangement 2012 may be used also for a single lumen cannula, preferably with or without a membrane.

FIG. 21 illustrates cannula system 2000 in a state in which an introducer 2114 stretches the cage arrangement 2012 for introducing cannula 2008 or 2010 into body 100. Introducer 2114 may also be named as mandrel. Introducer 2114 has a proximal end 2114p and a distal end 2114d. Proximal end 2114p may be clamped within locking mechanism 2002 in the position in which cage arrangement 2012 is stretched enabling the practitioner or the physician to fully concentrate on careful introduction of cannula system 2000 into body 100. Distal end 2114d may be adapted to engage with cage arrangement 2012, preferable with the distal tip and/or the distal portion of cage arrangement 2012. Distal end 2114d may be tapered. If cannula 2008, 2010 is in place, introducer 2114 is unlocked by operating locking mechanism 2002. Thereafter, introducer 2114 is pulled out of cannula 2008, 2010.

At the end of the medical treatment, introducer 2114 may be used again to stretch cage arrangement 2012 and to remove cannula 2008, 2010 out of body 100.

The diameter of introducer 2114 is adapted to have only small slit/gap between an outer surface of introducer 2114 and an inner surface of cannula 2008, 2010. The slit/gap may be smaller than 0.5 millimeter or smaller than 250 micron (micrometer). However, the slit/gap may be greater than 100 micron to allow axial movement of introducer 2114 within cannula 2008, 2010.

Alternatively, introducer 2114 may also be used for cannula system 1900 or for other cannula systems, for instance the cannula systems that are shown in FIGS. 1 to 14. Introducer 2144 may also be used for cage arrangements (diameter variable arrangements) that comprise a membrane, see for instance FIGS. 16 and 17. An adapted introducer may be used to introduce cannulas comprising cage arrangement 1800 that is shown in FIG. 18.

FIG. 22 illustrates an alternative embodiment of a circuitry 2206 wherein a cannula 2240a is pierced or punctured through a ventricle septum VS of heart H. Circuitry 2206 is extracorporeal. Circuitry 2206 may comprise or may consist of:
single lumen cannula 2240a, inserted preferably endovascular jugular,
a single lumen cannula 2240b, inserted preferably endovascular jugular,
a pump P22, and
an oxygenator OXY22.

A proximal end of cannula 2240b may be connected to an input port of oxygenator OXY22 via a flexible tube 2220. An output port of oxygenator OXY22 may be connected to an input port of pump P22, for instance via a tube 2240. An outlet port of pump P22 may be connected to a proximal end of cannula 2240a.

Cannula 2240a may carry a cage arrangement 2246 at its distal end. Cage arrangement 2246 may be placed within ascending aorta aAO. Cage arrangement 2246 may comprise a membrane, for instance a membrane having an opening that faces distally. Alternatively, cage arrangement 2246 may not have a membrane. Cannula 2240a is inserted endovascular through left jugular vein, superior vena cava SVC, right atrium RA, right ventricle RV, ventricle septum VS, left ventricle LV up to ascending aorta aAO. Especially cage arrangement 2246 may be placed within ascending aorta aAO. Only a smart part of the distal end of cannula 2240a may be located within cage arrangement 2246 and therefore also within ascending aorta aAO, for instance less than 5 mm Thus, cage arrangement 2246 does not comprise a separate distal tip (for instance made of a different material compared to the material of cannula 2240a) that has lateral side holes and/or a distal end-hole. However, in an alternative embodiment, cage arrangement 2246 may comprise a distal tip that has lateral side holes and/or a distal end-hole.

Cannula 2240b may carry a cage arrangement 2286 at its distal end. Cage arrangement 2286 may be placed within left atrium LA preferably transseptal through the atrial septum of the heart H. Cage arrangement 2286 may comprise a membrane, for instance a membrane having an opening that faces laterally. Alternatively, cage arrangement 2286 may not have a membrane. Cannula 2240b is inserted endovascular through right jugular vein, superior vena cava SVC, atrial septum AS into left atrium LA. Especially cage arrangement 2286 may be placed within left atrium LA. Only a smart part of the distal end of cannula 2240b may be located within cage arrangement 2286 and therefore also within left atrium LA, for instance less than 5 mm. Thus, cage arrangement 2286 does not comprise a separate distal tip (for instance made of a different material compared to the material of cannula 2240b) that has lateral side holes and/or a distal end-hole. However, in an alternative embodiment, cage arrangement 2286 may comprise a distal tip that has lateral side holes and/or a distal end-hole.

Alternatively, it is possible to insert cannula 2240a through right internal jugular vein rIJV and cannula 2240b through left internal jugular vein lIJV. Guide wires and/or introducer members may be used in all cases for the insertion of cannula 2240a and 2240b.

Pump P22 drives a drainage flow that comes in through all four pulmonary (see arrows 2297) veins PV out of left atrium LA, through cannula 2240b, tube 2220, oxygenator OXY22, pump P22, tube 2240 and finally through cannula 2240a into ascending aorta aAO, see arrow 2270. Pump P22 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P22 may generate a continuous blood flow.

Alternatively, cannula 2240a may be a dual lumen cannula or a multi lumen cannula. Cannula 2240b may be omitted if cannula 2240a is a dual lumen cannula or a single lumen cannula. If cannula 2240a is omitted and if cannula 2240 is a dual lumen cannula the outer cannula may be used to drain blood from left ventricle LV.

The ventricle septum VS may be a preferred place for puncturing, for instance if the atrial septum may not be used. Other medical devices may be placed within atrial septum or it may have been punctured too often. There may also be a disease of the atrial septum. However, even without special reasons the ventricle septum VS may be used and not the atrial septum.

The ventricle septum VS may be used for instance in variants of the following embodiments:
in the embodiment that is shown in FIG. 1 for cannula 110,
in the embodiment that is shown in FIG. 2 for cannula 210, a separate cannula may be used with inlet portion 250 in left atrium LA,
in the embodiment that is shown in FIG. 4 for cannula 410,
in the embodiment that is shown in FIG. 5 for cannula 510,
in the embodiment that is shown in FIG. 6 for cannula 640,
in the embodiment that is shown in FIG. 8 for cannula 840,
in the embodiment that is shown in FIG. 9 for cannula 910,
in the embodiment that is shown in FIG. 10 for cannula 1040,
in the antegrade variant of the embodiment that is shown in FIG. 10 for cannula 1040,
in the antegrade lobe dedicated variant of the embodiment that is shown in FIG. 10 for cannula 1040.

It is possible to avoid two cannulas within the right ventricle RV if the main pulmonary artery PA or especially the right pulmonary artery rPA or the left pulmonary artery lPA are reached transcaval from vena cava VC, especially from superior vena cava SVC by puncturing of the vena cava VC and of the respective pulmonary artery PA, see for instance FIG. 25. The cannula that takes the transcaval “short cut” may be inserted endovascular jugular, preferably through one of the interior jugular veins, e.g. left internal jugular vein lIJV or right internal jugular vein rIJV. However, endovascular femoral access to vena cava VC and then transcaval to one of the pulmonary arteries PA is possible as well. The transcaval cannula may be a single lumen cannula.

FIG. 23 illustrates a further alternative embodiment of a circuitry 2306 wherein a dual lumen cannula 2310 is pierced or punctured through ventricle septum VS. Circuitry 2306 is extracorporeal. Circuitry 2306 may comprise or may consist of:
multi lumen or dual lumen cannula 2310, inserted preferably endovascular jugular,
a pump P23, and
an oxygenator OXY23.

A proximal end of the outer cannula of dual lumen cannula 2310 may be connected to an input port of pump P23 via a flexible tube 2320. An output port of pump P23 may be connected to an input port of oxygenator OXY23, for instance via a tube 2340. An outlet port of oxygenator OXY23 may be connected to a proximal end of the inner cannula of dual lumen cannula 2310.

The inner cannula of dual lumen cannula 2310 may carry a cage arrangement 2346 at its distal end. Cage arrangement 2346 may be placed within ascending aorta aAO. Cage arrangement 2346 may comprise a membrane, for instance a membrane having an opening that faces distally. Alternatively, cage arrangement 2346 may not have a membrane.

Cannula 2310 may be a fixed dual lumen cannula or a non-fixed dual lumen cannula. A fixed dual lumen cannula 2310 is inserted endovascular through right jugular vein or left jugular vein, superior vena cava SVC, right atrium RA, right ventricle RV, ventricle septum VS, left ventricle LV up to ascending aorta aAO. Especially cage arrangement 2346 may be placed within ascending aorta aAO. Only a smart part of the distal end of the inner cannula of cannula 2310 may be located within cage arrangement 2346 and therefore within ascending aorta aAO, for instance less than 5 mm. Thus, cage arrangement 2346 does not comprise a separate distal tip (for instance made of a different material compared to the material of the inner cannula of dual lumen cannula 2310 that has lateral side holes and/or a distal end-hole. However, in an alternative embodiment, cage arrangement 2346 may comprise a distal tip that has lateral side holes and/or a distal end-hole.

The outer cannula of dual lumen cannula 2310 may have an inlet portion 2390 comprising a group of inlet holes, for instance a number of holes within the range from 4 to 20. Inlet portion 2390 may be placed within the right atrium RA if cannula 2310 is in its final position within heart H. Additionally or alternatively, the outer cannula of dual lumen cannula 2310 may have an inlet portion 2398 comprising a group of inlet holes, for instance a number of holes within the range from 4 to 20. Inlet portion 2398 may be placed within the right ventricle RV if cannula 2310 is in its final position within heart H.

There may be an optional cage arrangement around inlet portion 2390. An outer sheet member may be used to hold this cage arrangement in its closed or non-expanded state during insertion of dual lumen cannula. An optional cage arrangement may be used around inlet portion 2398. An outer sheet member may be used to hold this cage arrangement in its closed or non-expanded state during insertion of dual lumen cannula. If a fixed dual lumen cannula is used. For a non-fixed dual lumen cannula it is possible to use an introducer member to bring the cage arrangement around inlet holes 2398 in its non-expanded state.

Alternatively, if cannula 2310 is a non-fixed dual lumen cannula it is possible to insert outer cannula first, i.e. through left internal jugular vein lIJV or right internal jugular vein rIJV, through vena cava VC, right atrium RA up to left ventricle LV. After the insertion of outer cannula of dual lumen cannula 2310 inner cannula is inserted through outer cannula and then through ventricle septum VS, left ventricle and up to ascending aorta aAO.

Guide wires and/or introducer members may be used in all cases for the insertion of cannula 2310 in one step or in two single steps that are performed in sequence, i.e. first insertion of outer cannula and then insertion of inner cannula.

Pump P23 drives a drainage flow from right atrium (see arrow 2392) and/or from right ventricle RV (see arrow 2399) through outer cannula of dual lumen cannula 2310, tube 2320, pump P23, oxygenator OXY22, tube 2330 and finally through inner cannula of dual lumen cannula 2310 into ascending aorta aAO, see arrow 2370. Pump P23 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P23 may generate a continuous blood flow.

The ventricle septum VS may be a preferred place for puncturing, for instance if the atrial septum may not be used. Other medical devices may be placed within atrial septum or it may have been punctured too often.

The ventricle septum VS may be used for instance in instead of the embodiment that is shown in FIG. 3.

FIG. 24 illustrates an alternative embodiment of a circuitry 2406 wherein a cannula L15b may be punctured transcaval from vena cava VC to the aorta AO. Circuitry 2406 may comprise or consist of:
a single lumen cannula L15a,
single lumen cannula L15b,
a pump P15, and
an optional oxygenator OXY.

A proximal end of cannula L15a may be connected to an inlet port of pump P15, for instance via a flexible tube. An outlet port of pump P15 may be connected to the proximal end of cannula L15b, for instance via a flexible tube. An oxygenator OXY and/or a carbon dioxide removal unit and/or an adsorber/filter unit and/or another medical device may be included within circuitry 2406 at an appropriate location.

Cannula L15a may be a single lumen cannula that carries at least one expandable arrangement, for instance a cage arrangement, especially a cage arrangement as describes above. If a cage arrangement is used, a membrane may be used as well that is connected to the cage arrangement. However, at least one cage arrangement without a membrane may be used.

Cannula L15a may be inserted endovascular through left internal jugular vein lIJV or through right internal jugular vein rIJV or through another appropriate vessel. Cannula L15a is farther inserted through vena cava VC into the right atrium RA and/or into the right ventricle RV. An inlet portion comprising a group of inlet holes may be arranged within right atrium RA on cannula L15a. Alternatively or additionally, an inlet portion comprising a group of inlet holes may be arranged within right ventricle RV on cannula 15a.

Cannula L15b may be a single lumen cannula that carries an expandable arrangement, for instance a cage arrangement, especially a cage arrangement as describes above, or a balloon, see description of FIGS. 17 and 18 below. If a cage arrangement is used, a membrane may be used as well that is connected to the cage arrangement. The membrane may have an opening that faces distally with regard to the longitudinal axis of cannula L15b.

Cannula L15b may be inserted endovascular through left internal jugular vein WV or through right internal jugular vein rIJV or through another appropriate vessel. Cannula L15b is farther inserted through vena cava VC, especially through superior vena cava SVC, through a hole within the wall of vena cava VC, especially a hole in superior vena cava SVC, transcaval to a hole within ascending aorta aAO up to the ascending aorta aAO, where it is fixed for instance by the expandable arrangement mentioned above.

Pump P15 may drive a drainage flow from right atrium RA (see arrow) and/or from right ventricle RV through cannula L15a, pump P15 and finally through cannula L15b into ascending aorta aAO, see arrow. Pump P15 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P15 may generate a continuous blood flow.

Optional oxygenator OXY may increase the oxygen content of the blood extracorporeal. Thereby, carbon dioxide may be removed.

Alternatively a split tip cannula may be used that comprises both cannulas L15a and L15b.

The atrial septum AS and/or the ventricle septum VS may not be a preferred place for puncturing, for instance if other medical devices are placed within the atrial septum and/or ventricle septum or if one of these septa has or both have been punctured too often. There may also be a disease affecting one or both of the atrial septum and/or of the ventricle septum. Furthermore, the proposed transcaval shortcut from vena cava VC, preferably from superior vena cava SVC, to ascending aorta aAO may be used if the valves of heart H do not function properly any more, for instance because of a disease. However, even without special reasons the shortcut to the aorta may be chosen and not a way through one of the septa.

The following embodiments may be modified:

FIG. 2, cannula L15b may be used for inner cannula of dual lumen cannula 210, outer cannula of dual lumen cannula 210 may still be used as a single lumen cannula,

FIG. 3, cannula L15b may be used for inner cannula of dual lumen cannula 310, outer cannula of dual lumen cannula 210 may still be used as a single lumen cannula,

FIG. 10, cannula L15b may be used for inner cannula of dual lumen cannula 1040, outer cannula of dual lumen cannula 1040 may still be used as a single lumen cannula,

FIG. 10, antegrade variant, cannula L15b may be used for inner cannula of dual lumen cannula 1040, outer cannula of dual lumen cannula 1040 may still be used as a single lumen cannula,

FIG. 10, antegrade lobe-dedicated variant, cannula L15b may be used for inner cannula of dual lumen cannula 1040, outer cannula of dual lumen cannula 1040 may still be used as a single lumen cannula.

FIG. 25 illustrates a further alternative embodiment wherein a cannula 16b is punctured transcaval from vena cava VC to main pulmonary artery PA or to right pulmonary artery rPA or to left pulmonary artery lPA. Circuitry 2506 may comprise or consist of:
a single lumen cannula L16a,
single lumen cannula L16b,
a pump P16, and
an optional oxygenator OXY.

A proximal end of cannula L16a is connected to an inlet port of pump P16, for instance via a flexible tube. An outlet port of pump P16 may be connected to the proximal end of cannula L16b, for instance via a flexible tube. An oxygenator OXY and/or a carbon dioxide removal unit and/or an adsorber/filter unit and/or another medical device may be included within circuitry 2506 at an appropriate location.

Cannula L16a may be a single lumen cannula that carries at least one expandable arrangement, for instance a cage arrangement, especially a cage arrangement as describes above. If a cage arrangement is used, a membrane may be used as well that is connected to the cage arrangement. However, at least one cage arrangement without a membrane may be used.

Cannula L16a may be inserted endovascular through left internal jugular vein lIJV or through right internal jugular vein rIJV or through another appropriate vessel. Cannula L16a is farther inserted through vena cava VC into the right atrium RA and/or into the right ventricle RV. An inlet portion comprising a group of inlet holes may be arranged within right atrium RA on cannula L16a. Alternatively or additionally, an inlet portion comprising a group of inlet holes may be arranged within right ventricle RV on cannula L16a.

Cannula L16a may be a single lumen cannula that carries an expandable arrangement, for instance a cage arrangement, especially a cage arrangement as describes above.

Cannula L16b may be inserted endovascular through left internal jugular vein lIJV or through right internal jugular vein rIJV or through another appropriate vessel. Cannula L16b is farther inserted through vena cava VC, especially through superior vena cava SVC, through a hole within the wall of vena cava VC, especially a hole in superior vena cava SVC, transcaval to a hole within main pulmonary artery PA or to right pulmonary artery rPA or to left pulmonary artery lPA up to main pulmonary artery PA or to right pulmonary artery rPA or to left pulmonary artery lPA, where it is fixed for instance by the expandable arrangement mentioned above.

Cannula L16b may be a single lumen cannula that carries an expandable arrangement, for instance a cage arrangement, especially a cage arrangement as describes above, or a balloon, see description of FIGS. 17 and 18 below. If a cage arrangement is used, a membrane may be used as well that is connected to the cage arrangement. The membrane may have an opening that faces distally with regard to the longitudinal axis of cannula L16b.

Pump P16 may drive a drainage flow from right atrium RA (see arrow) and/or from right ventricle RV through cannula L16a, pump P16 and finally through cannula L16b into main pulmonary artery PA, right pulmonary artery rPA or left pulmonary artery, see arrow. Pump P16 may be operated in pulsed mode or may be a pump that generates a pulsatile blood flow, for instance a roller pump. Synchronization to the diastole and systole phases of heart pumping is possible if a sensor is used, for instance a blood pressure sensor. Alternatively, blood pump P16 may generate a continuous blood flow.

Optional oxygenator OXY may increase the oxygen content of the blood extracorporeal. Thereby, carbon dioxide may be removed.

Alternatively a split tip cannula may be used that comprises both cannulas L16a and L16b.

The atrial septum AS and/or the ventricle septum VS may not be a preferred place for puncturing, for instance if other medical devices are placed within the atrial septum AS and/or ventricle septum VS or if one of these septum or has or both have been punctured too often. There may also be a disease affecting one or both of the atrial septum AS and/or of the ventricle septum VS. Furthermore, the proposed transcaval shortcut from vena cava, preferably from superior vena cava SVC, to main pulmonary artery PA, to right pulmonary artery rPA or to left pulmonary artery may be used if the valves of heart H do not function properly any more, for instance because of a disease. However, even without special reasons shortcut to the pulmonary artery (PA, rPA, lPA) may be chosen and not a way through one of the septa AS, VS.

The following embodiments may be modified for instance:
in the embodiment that is shown in FIG. 6, i.e. in a pECLA (percutaneous extra corporeal lung assist) circuitry 606, cannula L16b may be used for a single lumen cannula 610 that extends from vena cava VC through right atrium RA and right ventricle into main pulmonary artery PA,
in the embodiment that is shown in FIG. 9 cannula L16b may be used for cannula 910, cannula L16a is not necessary,
in the embodiment that is shown in FIG. 10 cannula L16b may be used for inner cannula of dual cannula 1010, the inlet portion(s) 1090 and/or 1098 of outer cannula of cannula 1010 may still be used, cannula L16a is not necessary,
in the antegrade variant of the embodiment that is shown in FIG. 10 cannula L16b may be used for inner cannula of dual cannula 1010, the inlet portions 1090 and/or 1098 of outer cannula of cannula 1010 may still be used, cannula L16a is not necessary, and
in the antegrade lobe-dedicated variant of the embodiment that is shown in FIG. 10 cannula L16b may be used for inner cannula of dual for cannula 1010, the inlet portion s 1090 and/or 1098 of outer cannula of cannula 1010 may still be used, cannula L16a is not necessary.

FIG. 26 illustrates a cannula L17 that carries an inflatable expandable arrangement Ba, for instance a balloon. Balloon Ba may have a cylindrical shape and may be connected to a distal portion of cannula L17, for instance using an adhesive.

A channel CH1 may be arranged on an outer surface of cannula L17 Channel CH1 may extend from a proximal part of cannula L17 up to balloon Ba. If a fluid is driven into channel CH1 balloon Ba inflates. If the fluid is driven out of channel CH1 then balloon Ba deflates.

Thus, balloon Ba may form a border element that is between cannula L17 and a vessel V of the blood circuit. Vessel V may be a pulmonary artery of lung L or a pulmonary vein of lung L. There may be a transport volume TrV that is used to treat lung L and that is on the distal side of inflated balloon Ba. The natural blood circuit BC may be on the proximal side of balloon Ba. Balloon Ba may isolate the natural blood circuit BC from transport volume TrV.

Transport volume TrV is directly in fluidic connection with cannula L17 through the holes in a separate distal tip Ti17, see for instance holes Ho17. Alternatively, cannula L17 may have only a single end-hole EH at its distal end.

Cannula L17 may be one of the cannulas mentioned above during the description of FIGS. 1 to 11 and 15 to 23.

Alternatively and/or additionally, channel CH1 may be arranged within cannula L17. Cannula L17 may be a single lumen cannula or a multi lumen cannula, especially the inner cannula or the outer cannula of a dual lumen cannula. A combination of an internal channel and an external channel is possible as well.

Deflated balloon Ba may not have a further protection shield during insertion of cannula L17. However, alternatively a removable sheath may be wrapped around balloon Ba during insertion of cannula L17.

FIG. 27 illustrates a split tip cannula L18 that carries two expandable arrangements on its two distal end lumens L18a and L18b. An inner lumen of cannula L18 bifurcates in distal end lumen L18a and distal end lumen L18b at a bifurcation point or bifurcation position Bi. Each of the expandable arrangements may be a balloon Ba as shown in FIG. 17 and described above. A channel CH2 may correspond to channel CH1 mentioned above Channel CH2 may be connected to a channel CH2a that extends on distal end lumen L18a to the respective balloon and to a channel CH2b that extends on distal end lumen L18b to the respective balloon. Alternatively or additionally, internal channels may be used to inflate or deflate the balloons. A combination of an internal channel and an external channel is possible as well. Furthermore, it is possible to use two separate channels CH2a1 and CH2b1 that extend from a proximal part of cannula L18 to either distal end lumen L18a or distal end lumen L18b. Separate and independent control of balloon on distal end lumen L18a and on distal end lumen L18b is possible in this variant. Introduction and fixation of cannula L18 is easier with separate control of both balloons.

Alternatively, two cage arrangements may be used on the distal end lumens L18a and L18b. Cage arrangements that are mentioned above may be used, see for instance FIGS. 1 to 11, 15 to 23. An introducer member 115 may be used that has a split tip, i.e. a bifurcation. Alternatively, two separate introducer members may be used within cannula L18, one extending into distal end lumen L15a and the other extending into distal end lumen L18b.

Cannula L18 may be a single lumen cannula having a split tip. Alternatively, cannula L18 may comprise two separate lumens, one connected to lumen L18a and the other connected to lumen L18b.

Furthermore, for each embodiment of cannula L18 described above cannula L18 may not be inserted into a further cannula or may be inserted in a further dual lumen cannula, for instance in a fixed dual lumen cannula or multi lumen cannula or non-fixed cannula dual lumen cannula or multi lumen cannula.

The treatment fluid flow may be heated in order to improve the uptake of medicaments/treatment substances by the tissue of the organ and/or by the cells of the organ. If the treatment fluid comprises blood or a high percentage of blood or blood components the heating temperature may be for instance in the range between 39.0 and 44.0° C. (degrees of Celsius), preferably to between 40.0 and 42.5° C.

However, due to the isolation of the transport volume from the body fluid and/or due to the local treatment even higher temperatures may be used, especially if the fluid flow through the transport volume does not contain or comprise blood or blood components or only a lower percentage of blood per volume. Therefore, also temperatures above 42.5° C. may be used, for instance above normal blood temperature, above 43° C., above 44° C., above 45° C. or even above 50° C. This may improve the uptake of medicaments/treatment substances further.

The term “normal body temperature (also known as normothermia or euthermia)”, as used herein, may refer to the typical temperature found in an individual. In humans, the normal body temperature is 37° C. This value is, however, only an average. The normal body temperature may be slightly higher or lower. A number of factors can influence the body temperature, including age, sex, time of day, and activity level. In babies and children, for example, the average body temperature ranges from 36.6° C. to 37.2° C. Among adults, the average body temperature ranges from 36.1° C. to 37.2° C. The normal human body temperature range is, thus, typically stated as being between 36.1° C. and 37.5° C., e.g. 36.1, 36.2, 36.3, 36.4, 36.5, 36.6, 36.7, 36.8, 36.9, 37.0 37.1, 37.2, 37.3, 37.4, or 37.5° C., in humans.

Furthermore, it is possible to use in all embodiments that are mentioned above an inner surface of the lumen portion and/or inner lumen that comprises a spirally and/or helically surface structure. The spirally and/or helically surface structure may have the effect that the fluid flow within the cannula is rotated as it moves through the cannula. Turbulences may be reduced thereby and/or it may be possible to reach much higher flow rates compared to cannulas that have a smooth inner surface, i.e. that do not have spirally and/or helical surface structures on their inner surfaces. However, it is of course possible to use cannulas without a spirally and/or helical surface features, if for instance lower flow rates are necessary. The spirally turned flow and/or the rotated flow may prevent clotting of blood cells if the fluid flow comprises blood, especially in slow flow rate conditions. However, there may also be advantages if the fluid flow does not contain blood. The spiral flow may be a laminar spiral flow.

There may be an embodiment in which a single lumen cannula or a dual or multi lumen cannula is used (fixed or non-fixed) wherein the single lumen cannula or the inner cannula of the dual or multi lumen cannula may have a split tip. Each distal tip of the split tip cannula may be associated with or may be carry an expandable arrangement, for instance a balloon or a cage, especially with a cage that carries a membrane. The distal parts of the split tip cannula may be inserted into the left pulmonary veins whereby the right pulmonary veins may be left open. Alternatively, the distal parts of the split tip cannula may be inserted into the right pulmonary veins whereby the left pulmonary veins may be left open.

Furthermore, there may be an embodiment in which a single lumen cannula or a dual cannula is used (fixed or non-fixed) wherein the single lumen cannula or the inner cannula of the dual lumen or multi lumen cannula may have a cage arrangement on its distal end. The cage arrangement may carry a membrane. The membrane may define an opening that faces laterally. If the cannula is inserted into the body, the opening of the membrane may face laterally in the direction of both right pulmonary veins. Both left pulmonary veins may remain open, i.e. blood may pass the outside of the membrane thus not entering the cannula that carries the cage arrangement with the membrane.

The following feature combinations may be relevant:

a) All embodiments that relate to a cage arrangement or to another expandable arrangement may be used to reach a stable and/or secure positioning or fixation of the cannula in the chambers of the heart (left atrium LA, right atrium RA, left ventricle LV, right ventricle RV) or vessels of the blood circuit. The cage arrangement or another expandable arrangement may allow a better design of the cannula, especially of the distal tip, for instance only one end-hole may be used instead of multi-hole distal parts. This may result in better or optimal flow characteristics, for instance less shear stress, less turbulences, etc. The cannula comprises at least one end hole or a single end-hole through which at least 25 volume percent, at least 50 volume percent, at least 75 volume percent or at least 90 volume percent or all of the flow flows into or out of the cannula, all volumes measured for instance for 3.5 l per minute or for 5 l per minute. The given volume percent of flow through the at least one end-hole may be measured for instance for a flow through the cannula of 3.5 l per minute or for 5l per minute. Thus, a significant portion of the flow flows through the at least one end hole. Additionally, the cannula may be a “tip-less” cannula which extends not or only at most 3 mm (millimeter) within the inner volume of the cage in the expanded state. Thus, the fixation of the cage may extend only up to the distal end of the cannula or up to a location which is equal to 3 mm or at most 3 mm away from the distal end of the cannula.

In another embodiment at least one side hole may be present in the cannula in which at least 25 volume percent, at least 50 volume percent, at least 75 volume percent or at least 90 volume percent or all of the flow flows into or out of at least one end-hole of the cannula and/or in the case of a tip-less cannula having an axial oriented inlet or outlet. The at least one side hole may have at least one other function in addition to the function of flow transport into or out of the cannula. The flow transport through the at least one side-hole may be equal to or less than 50 volume percent of the overall flow through the cannula or equal to or less than 25 volume percent of the overall flow through the cannula. The given volume percent of flow through the at least one side hole may be measured for instance for a flow through the cannula of 3.5 l per minute or for 5l per minute. As is mentioned below, known cannulas, for instance TandemHeart® ProtekSolo® may transport less than 12 volume percent through a single end-hole. However, the known cannula does not comprise an expandable arrangement, especially no cage arrangement.

b) All embodiments may be used with fix or non-fixed dual lumen cannulas or multi lumen cannulas. If non-fixed (may be inserted into each other) cannulas are used, it is easier to position or implant the cannulas stepwise along a curved introduction path because less friction may be involved, especially at the puncture site, and the insertion may be less traumatic.

c) All embodiments may be used for endovascular access and for in-vivo lung isolation which may allow an isolated perfusion of lung L or of parts of lung L, for instance in a closed circuit of fluid flow that may be isolated from the body blood circuit.

d) If within the cage a cannula tip is used which has side-holes it is possible to have at least one end-hole or to have no end-holes.

e) The cannula with a cage may be used for other application scenarios, especially medical application scenarios. Applications within the blood or vessel network, are for instance applications in one the veins or within one of the arteries. Thus, the cage and/or the membranes may be used within the descending and lower aorta, especially on or at the branching of vessels to the bowels and to the kidney or at the artery branching to the liver. An example for a vein application, is the vein in which the veins coming from the bowels, kidney or liver lead to, especially for fixation at the junction or bifurcation of this vein. Furthermore, applications within other organs are possible, i.e. brain, liver, kidney, etc. Human applications are possible as well as applications in animals.

The combination of at least two arbitrarily selected or of all feature combinations a), b), c), d) and e) may give the best result.

Moreover, the cannula, for instance the delivery cannula, may be inserted endovascular jugular and may be punctured from superior vena cava SVC or from right atrium RA transcaval to ascending aorta aAO. Alternatively, the cannula, for instance the delivery cannula, may be inserted endovascular femoral through inferior vena cava IVC into the right atrium RA and may be punctured from superior vena cava SVC or from right atrium RA transcaval to ascending aorta aAO.

Moreover, the delivery cannula or the drainage cannula may be inserted endovascular jugular and may be punctured from superior vena cava SVC or from right atrium RA transcaval to the main pulmonary artery PA or to the right pulmonary artery rPA or in special cases to the left pulmonary artery lPA. Alternatively, the delivery cannula or the drainage cannula may be inserted endovascular femoral through inferior vena cava IVC into the right atrium RA and may be punctured from superior vena cava SVC or from right atrium RA transcaval to the main pulmonary artery PA or to the right pulmonary artery rPA or in special cases to the left pulmonary artery lPA.

In all embodiments with a cage arrangement it is also possible to use another material than a metal, for instance a natural and/or biological material, especially cellulose, for instance cellulose that is treated to increase the hardness. Compatibility with body 100 and/or with blood may be improved thereby.

Furthermore, it is possible to use in all embodiments that are mentioned above an inner surface of the lumen portion and/or inner lumen that comprises a spirally wound surface structure. The spirally wounding may have the effect that the fluid flow within the cannula is rotated as it moves through the cannula. Turbulences may be reduced thereby and/or it may be possible to reach much higher flow rates compared to cannulas that have a smooth inner surface, i.e. that do not have spirally wound structures on their inner surfaces. However, it is of course possible to use cannulas without spirally wound features if lower flow rates are necessary.

1. In the following simulation results are presented:

Content:

I. Project overview (section 2.)

II. Simulation setup (section 3. to 8.)

III. Results/discussion
A. Left atrial vortex (section 9. to 11.)
B. Flow into the TandemHeart® cannula (section 12. to 14.)
C. Steady state flow pattern (section 15. to 19.)
D. Velocity distribution (section 20. to 25.)
E. Wall shear stress (section 26. to 32.)
F. Pressure distribution and pressure loss (section 33. to 39.)
G. Left atrial appendix flow (section 40. to 41.)

IV. Overall conclusion (section 42. and 43.)

V. References (section 44.)

Short Summary:

The simulation showed that the “end-hole, tip-less” ReCO₂Lung® cannula enables:
Less wall shear stress,
Less shear rates,
Less turbulences,
Less recirculation, and
More homogeneous velocity profile.

If the end-hole is positioned in the left atrium, the natural blood flow in the atrium is not hindered compared to the placement of a tip with holes within the left atrium. Furthermore, a tip with side holes may promote the formation of thrombus on the small holes. This is not the case if only one end-hole is used.

Thus, it may be advantageous to combine all embodiments and examples mentioned in this description with a tip-less cannula having only one end hole and having an outer diameter of 21 French or more, especially of 23 Fr, 25 Fr, 27 Fr, 29 Fr and 31 Fr. The end hole is preferably located at the proximal end of the inner volume defined by an optional cage or within a distance of 5 mm (millimeters) or less than 5 mm, of 4 mm or less than 4 mm, of 3 mm or less than 3 mm, 2 mm or less than 2 mm or 1 mm or less than 1 mm from the proximal end of the inner volume defined by an optional cage.

### I. PROJECT OVERVIEW

2. Project Overview:
Steady-state simulation, see section III., C,
Investigation of the flow in the left atrium as well as in and around the transseptal applied cannula, see section III.,
Parameters:
   Cannula type
   Cannula diameter
   Outflow
   Atrial geometry
Aim: Comparison of TandemHeart® ProtekSolo® with ReCO₂Lung® cannulas of several sizes at different operation points, i.e. with cannulas according to embodiments of the present application/patent.

### II. SIMULATION SETUP

3. Simulation Setup—Basic Setting:

Cannula Geometries:

TandemHeart® ProtekSolo®: 21 Fr (French), as described in patent US 2011 0160517 A1 [1] which is incorporated by reference herewith and as illustrated in FIG. 28,
Lumina (ReCO₂lung):

| | | | | | | |
|---|---|---|---|---|---|---|
| Outer | 21 | 23 | 25 | 27 | 29 | 31 |
| Diameter (Fr) | | | | | | |
| Inner Lumen | 17.83 | 19.92 | 21.81 | 23.91 | 26.01 | 27.81 |
| (Fr) | | | | | | |

Atrial volume (related to the atrial cycle or of the cardiac cycle):
   End-diastolic configuration: approx. 63 mL
   End-systolic configuration: approx. 20 mL
   Volumes according to ranges stated in literature [2], [3], [4], [5] which are incorporated by reference herewith
Blood specifications:
   Shear dependent viscosity model
   Density: 1.0564 kg/l (Ht=0.44), hematocrit Ht, i.e. volume percentage of red blood cells (RBC)

As is illustrated in FIG. 28, a TandemHeartR cannula 2800 comprising a cannula tip 2824 is used as a reference cannula which is inserted transseptal. The outer diameter DO1 is 21 Fr. The flow rate is for instance 3.5 liter per minute. This reference cannula tip 2824 is compared with cannulas having only one end-hole and which are tip-less and have outer diameters DO1 of 21 Fr, 23 Fr, 25 Fr, 27 Fr, 29 Fr and 31 Fr (French). The flow rates for the tip-less cannulas were for instance 3.5 liter per minute or 5 liter per minute. Tip-less means that there are no side holes or only side holes which transport less than 25 percent of the overall flow, measured for instance for a flow of 3.5 liter per minute or 5 liter per minute.

The distal tip 2824 of the reference cannula 2800 comprises:
a wire enforced portion 2816 which forms a wall and which extends proximally along the whole cannula 2800,
a preferably flat wire FW/2822 that forms portion 2816,
a channel 2814 that is formed within wire enforced portion 2816,
for instance, 14 side holes, see for instance hole 2826, see chapter B., section 12. below,
for instance, one (1) end hole, see chapter B., section 12. below, and
distal markers 2832, for instance three markers, e.g. made of tantalum.

The following dimensions may be used:
Outer diameter DO1 of wire enforced portion 2816 may be 0.274 inch+/−0.07 inch/0.013 inch,
Outer diameter DO2 of cannula tip 2824 at distal end of tapered portion may be equal to 0.186 inch+1-0.003 inch,
Inner diameter DI1 of cannula (main portion) of 0.232 inch (0.59 cm)+/−0.003 inch,
Inner diameter DI2 of holes, especially of side hole 2826 may be 0.094 inch+/−0.010 inch/0.018 inch, at 14 PLCS (places), i.e. for all 14 side holes,
Inner diameter DI 3 of distal marker 2832 may be equal to 0.050 inch+/−, 3 PLCS/places),
Distance Di1 in longitudinal direction between end of wire enforced portion 2816 and distal end of cannula tip 2824 may be equal to 1.15 inch+/−0.080 inch/0.150 inch,
Distance Di2 in longitudinal direction between center of proximal hole and distal end of cannula tip 2824,
Distance Di3 in longitudinal direction between center of proximal hole and center of distal hole may be equal to 0.564 inch+/−0.60 inch,
Distance Di4 between proximal end of conical/tapered portion and distal end of conical/tapered portion in longitudinal direction may be in the range of 0.20 inch to 0.25 inch,
Distance Di5 in longitudinal direction between end of wire enforced portion and center of proximal hole of tip 2824 may be equal to 0.185 inch+/−0.060 inch,
Distance Di6 between center of hole 2832 and distal end of cannula tip 2824 may be 0.121 inch+/−0.030 inch,
Thickness Th1 of front wall which encloses an end hole of tip 2824 distally may be at most (maximum) 0.020 inch.
Radius R2 of distal edge may be 0.20 inch,
Flat wire FW/2822 may have a height of 0.004 inch and a width of 0.20 as well as 0.37+/−1 TPI (Treads per inch).

4. Simulation Setup—Mesh:

See FIG. 29. A length of about 1 cm (centimeter) is shown in FIG. 29.
Fine resolution of near-wall flow, for instance
   10 prism layers
   Initial prism layer height 0.03 mm
Mesh size, for instance
   1.2-2.5 million elements
   0.4-0.6 million nodes

5. Simulation Setup—Boundary Conditions:

A length of about 4 cm (centimeter) is shown in FIG. 30.
Boundary conditions, for instance:
   6 mmHg (millimeter mercury column) at the pulmonary veins
Cannula outflow:
   3.5 l/min (liter per minute) (TandemHeart® and ReCO₂Lung® 21 Fr to 27 Fr)
   5 l/min (TandemHeart® and ReCO₂Lung® 29 Fr, 31 Fr)

6. Simulation Setup—Cannula Position
Anterior view, depth of the cannula inside the atrium

ReCO₂Lung®: FIGS. 31A and 31C

TandemHeart®: FIGS. 31B and 31D

End-systolic: FIGS. 31A and 31B

End-diastolic: FIGS. 31C and 31D

It is apparent from FIGS. 31A to 31 D that volume change of left atrium due to heart beat was simulated.

The depth of the ReCO₂Lung® cannula inside the left atrium was 3 mm in both periods of the heartbeat, i.e. systolic and diastolic. The ReCO₂Lung® cannula comes from top, e.g. it may be inserted via the right jugular vein.

The depth of the TandemHeartR cannula inside the left atrium was 30 mm in both periods of the heartbeat. The TandemHeart® cannula comes from below, e.g. it may be inserted via the right femoral vein.

7. Simulation Setup—Cannula Position
Lateral view

ReCO₂Lung®: FIGS. 32A and 32C

TandemHeart®: FIGS. 32B and 32D

End-systolic: FIGS. 32A and 32B

End-diastolic: FIGS. 32C and 32D

8. Simulation Setup—Limitations
Steady-state simulations . . .
   can only provide information about the flow field at the simulated time points. In this project, only the flow field at the end of the atrial systole and the end of the atrial diastole was investigated.
   neglect the influence of flow features, which occur at other points during the cardiac cycle and which may impact the flow at the simulated time point.
   neglect the specific movement of the atrial wall, which has a notable impact on the flow field. However, the start position of the movement and the end position of the movement are considered.
The anatomy . . .
   is an example of a human left atrium and has been chosen carefully to (1) not to represent an abnormal case and (2) still reflect the wide volume range between different persons. Patient anatomies vary significantly and conclusions drawn from these simulations will not necessarily apply to all left atrial anatomies.

### III. RESULTS AND DISCUSSION

A. Left Atrial Vortex:

9. Results—Left Atrial Vortex
Left atrial vortex present in all cases as reported in literature

ReCO₂Lung®: FIGS. 33A and 33C

TandemHeart®: FIGS. 33B and 33D

End-systolic: FIGS. 33A and 33B

End-diastolic: FIGS. 33C and 33D

FIG. 33E: Illustrated is a flow pattern in an early phase of the diastolic period of flow MRI of real heart for comparison reasons.

FIG. 33F: Illustrated is a flow pattern in a middle phase of the diastolic period of flow MRI of real heart for comparison reasons.

FIG. 33G: Illustrated is a flow pattern in a late phase of the diastolic period of flow MRI of real heart for comparison reasons.

FIGS. 33E, 33F and 33G are mentioned in reference [6], see section 44. below.

10. Results—Flow Around the Cannula
Circumferential flow pattern around the cannula tips at simulated time point

See FIGS. 34A to 34G.

ReCO₂Lung®: FIGS. 34A and 34C

TandemHeart®: FIGS. 34B and 34D

End-systolic: FIGS. 34A and 34B

End-diastolic: FIGS. 34C and 34D

FIG. 34E: Location of cross section that is illustrated in FIGS. 34A and 34B (end-systolic).

FIG. 34F: Location of cross section that is illustrated in FIGS. 34C and 34D (end-diastolic).

11. Discussion—Flow Around the Cannula
Studies suggest that the left atrial vortex provides benefits regarding atrial washout, energy conservation and flow guidance towards the mitral valve, see references [6] and [7] as mentioned below in section 44.
The influence of the cannulas on the left atrial vortex appears to be limited at the examined points in the cardiac cycle.
The results show a circumferential flow around the cannula tips at both examined points in the cardiac cycle.

Washout in the cannula insertion region can be expected for both ReCO₂Lung® and TandemHeart® cannula.

B. Flow into the TandemHeart® Cannula:

12. Results—Flow into the TandemHeart® Cannula
All settings exhibit about the same inflow distribution.
Most of the flow enters through the two proximal side holes (about 12 percent each) and the end hole (about 11 percent).

FIG. 35A illustrates the flow through each opening of the reference cannula TandemHeart® within a Cartesian coordinate system. The horizontal x-axis of the coordinate system illustrates the number of the hole as is illustrated in FIG. 35B. The vertical y-axis of the coordinate system illustrates the percentage of the total flow through the respective hole as indicated on the x-axis.

ED—end diastolic (dark blue, first column in each column group for respective hole): 3.5 l/min (liter per minute)

ED—end diastolic (light blue, second column in each column group for respective hole): 5l/min

ES—end systolic (dark green, third column in each column group for respective hole): 3.5 l/min

ES—end systolic (light green, fourth column in each column group for respective hole): 5l/min

FIG. 35B illustrates cannula tip 2824 comprising 14 side holes, e.g. hole numbers 1 to 14 and one end hole, e.g. hole number 15. Holes with hole numbers 1 and 2 are the most proximal side holes. Holes with hole numbers 1, 5, 9 and 13 are at the right side of cannula tip 2824. Holes with hole numbers 2, 6, 10 and 14 are on the left side. Holes with hole numbers 3, 7 and 11 are on the top of cannula 2824. Holes with hole numbers 4, 8 and 12 are at the bottom side of cannula tip 2824.

13. Results—Flow into the TandemHeart® Cannula
The flow in the cannula gradually increases in the TandemHeart® cannula.

FIG. 36A illustrates the overall flow through the reference cannula TandemHeart® within a Cartesian coordinate system. The horizontal x-axis of the coordinate system illustrates the x-position in accordance with the x-position as illustrated in FIG. 36B, i.e. two vertical lines correspond to the left side of the hole with hole number 2 and to the right side of the hole with hole number 14. The vertical y-axis of the coordinate system illustrates the total flow through cannula 2824 at the corresponding x-position within the range of 0 liter per minute to 5 liter per minute.

ED—end diastolic (dark blue, lower curve): 3.5 l/min (liter per minute)

ED—end diastolic (light blue, upper curve): 5l/min

ES—end systolic (dark green, same as lower curve): 3.5 l/min

ES—end systolic (light green, same as upper curve): 5l/min

FIG. 35B illustrates a cross section of cannula tip 2824 including the longitudinal axis of cannula tip 2824. Holes with hole numbers 2, 6, 10 and 14 are illustrated from left to right. Inflow is on the right side of cannula tip 2824. As is apparent from FIG. 36A a flow of about 0.4 liter flows through the end hole in the 3.5 liter per minute examples. A flow of about 0.6 liter enters through end hole (number 15) in the 5 liter per minute examples. Both curves are exponentially decreasing in the region between the two vertical lines.

14. Discussion—Flow into the TandemHeart® Cannula
The flow does not enter equally through all openings, but mostly from the two most proximal side holes and the end hole (collectively over 35%). This applies to all four simulated settings.
The large number of side holes facilitated a gradual increase in cannula flow. This might help to reduce high velocity gradients in some areas. The following sections will discuss the differences between TandemHeart® and ReCO₂Lung® cannula regarding the velocity distribution.

C. Steady-State Flow Pattern:

15. Results—Steady-State Flow Pattern
End-systolic configuration

ReCO₂Lung®, 3.5 l/min: FIGS. 37A to 37D, respectively 21 Fr, 23 Fr, 25 Fr and 27 Fr

ReCO₂Lung®, 5 l/min: FIGS. 37E and 37F, respectively 29 Fr and 31 Fr

TandemHeart®: FIGS. 37G and 37H, each 21 Fr but FIG. 37G for 3.5 l/min and FIG. 37H for 5l/min

The velocity is in the range from 0 meter per second to about 3.0 meter per second.

16. Results—Steady-State Flow Pattern
End-diastolic configuration

ReCO₂Lung®, 3.5 l/min: FIGS. 38A to 38D, respectively 21 Fr, 23 Fr, 25 Fr and 27 Fr

ReCO₂Lung®, 5 l/min: FIGS. 38E and 38F, respectively 29 Fr and 31 Fr

TandemHeart®: FIGS. 38G and 38H, each 21 Fr but FIG. 38G for 3.5 l/min and FIG. 38H for 5l/min

The velocity is in the range from 0 meter per second to about 3.0 meter per second.

17. Results—Flow at the Cannula Tip, 3.5 l/Min
Similar velocities in the inflow tract, similar flow pattern in the atrium

ReCO₂Lung®, 21 Fr: FIGS. 39A and 39C

TandemHeart®, 21 Fr: FIGS. 39B and 39D

End-systolic: FIGS. 39A and 39B

End-diastolic: FIGS. 39C and 39D

The velocity is in the range from 0 meter per second to about 3.0 meter per second.

18. Results—Flow at the Cannula Tip, 5l/Min
Similar velocities in the inflow tract, similar flow pattern in the atrium

ReCO₂Lung®, 29 Fr: FIGS. 40A and 40C

TandemHeart®, 21 Fr: FIGS. 40B and 40D

End-systolic: FIGS. 40A and 40B

End-diastolic: FIGS. 40C and 40D

The velocity is in the range from 0 meter per second to about 3.0 meter per second.

19. Results—Flow at the Cannula Tip, 5l/Min
ReCO₂Lung® cannula smaller velocity gradient and lower velocities

ReCO₂Lung®, 31 Fr: FIGS. 41A and 41C

TandemHeart®, 21 Fr: FIGS. 41B and 41D

End-systolic: FIGS. 41A and 41B

End-diastolic: FIGS. 41C and 41D

The velocity is in the range from 0 meter per second to about 3.0 meter per second.

D. Velocity Distribution:

20. Results—End-Systolic Cannula Flow
Steady-state velocity contours show differences depending on flow and diameter

ReCO₂Lung®, 3.5 l/min: FIGS. 42A to 42D, respectively 21 Fr, 23 Fr, 25 Fr and 27 Fr

ReCO₂Lung®, 5 l/min: FIGS. 42E and 42F, respectively 29 Fr and 31 Fr

TandemHeart®: FIGS. 42G and 42H, each 21 Fr but FIG. 42G for 3.5 l/min and FIG. 42H for 5l/min

The velocity is in the range from 0 meter per second to about 3.0 meter per second.

21. Results—End-Diastolic Cannula Flow
Steady-state velocity contours show differences depending on flow and diameter

ReCO₂Lung®, 3.5 l/min: FIGS. 43A to 43D, respectively 21 Fr, 23 Fr, 25 Fr and 27 Fr

ReCO₂Lung®, 5 l/min: FIGS. 43E and 43F, respectively 29 Fr and 31 Fr

TandemHeart®: FIGS. 43G and 43H, each 21 Fr but FIG. 43G for 3.5 l/min and FIG. 43H for 5l/min

The velocity is in the range from 0 meter per second to about 3.0 meter per second.

22. Results—Cannula Flow, 3.5 l/Min
Similar velocity, but more homogeneous velocity profile in the ReCO₂Lung® cannula

ReCO₂Lung®, 21 Fr: FIGS. 44A and 44C

TandemHeart®, 21 Fr: FIGS. 44B and 44D

End-systolic: FIGS. 44A and 44B

End-diastolic: FIGS. 44C and 44D

The velocity is in the range from 0 meter per second to about 3.0 meter per second.

23. Results—Cannula Flow, 5 l/Min
Similar velocity, but more homogeneous velocity profile in the ReCO₂Lung® cannula

ReCO₂Lung®, 21 Fr: FIGS. 45A and 45C

TandemHeart®, 21 Fr: FIGS. 45B and 45D

End-systolic: FIGS. 45A and 45B

End-diastolic: FIGS. 45C and 45D

The velocity is in the range from 0 meter per second to about 3.0 meter per second.

24. Results—Cannula Flow, 5l/Min
Lower velocity and more homogeneous velocity profile in the larger ReCO₂Lung® cannula

ReCO₂Lung®, 29 Fr: FIGS. 46A and 46C

TandemHeart®, 21 Fr: FIGS. 46B and 46D

End-systolic: FIGS. 46A and 46B

End-diastolic: FIGS. 46C and 46D

The velocity is in the range from 0 meter per second to about 3.0 meter per second.

25. Discussion—Steady-State Velocity Profiles
The velocity range is similar in both ReCO₂Lung® and TandemHeart® cannula when diameter and flow are the same.
Higher velocities occur in cannulas with smaller diameter and/or at high flow rates. They induce higher velocity gradients and higher shear rates, which are not desirable. Small velocities and velocity gradients on the other hand occur in cannulas with larger diameter.
The possibility to use a larger lumen cannula is a significant benefit of the ReCO₂Lung® cannula compared to the TandemHeart® cannula and is most relevant in the range of higher flow rates.
Due to the side holes, the velocity profile in the TandemHeart® cannula is less homogeneous, increasing the areas of higher velocity gradients and shear rates.
High velocity gradients towards the wall induce high wall shear stresses and are generally not desirable in this case. The wall shear stress analysis will be presented in the following sections.

E. Wall Shear Stress:

26. Results—Steady-State Wall Shear Stress, End-Systolic
Bigger areas of high wall shear stress in the TandemHeart® cannula

ReCO₂Lung®, 3.5 l/min: FIGS. 47A to 47D, respectively 21 Fr, 23 Fr, 25 Fr and 27 Fr

ReCO₂Lung®, 5 l/min: FIGS. 47E and 47F, respectively 29 Fr and 31 Fr

TandemHeart®: FIGS. 47G and 47H, each 21 Fr but FIG. 47G for 3.5l/min and FIG. 47H for 5l/min

The wall shear stress is in the range from 0 Pa (Pascal) to about 50.0 Pa.

27. Results—Steady-State Wall Shear Stress, End-Diastolic
Bigger areas of high wall shear stress in the TandemHeart® cannula

ReCO₂Lung®, 3.5 l/min: FIGS. 47A to 47D, respectively 21 Fr, 23 Fr, 25 Fr and 27 Fr

ReCO₂Lung®, 5 l/min: FIGS. 47E and 47F, respectively 29 Fr and 31 Fr

TandemHeart®: FIGS. 47G and 47H, each 21 Fr but FIG. 47G for 3.5 l/min and FIG. 47H for 5l/min

The wall shear stress is in the range from 0 Pa (Pascal) to about 50.0 Pa.

28. Results—Wall Shear Stress, TandemHeart® Cannula
High wall shear stress regions, top view

TandemHeart®, 3.5 l/min: FIGS. 49A and 49C

TandemHeart®, 3.5 l/min: FIGS. 49B and 49D

End-systolic: FIGS. 49A and 49B

End-diastolic: FIGS. 49C and 49D

The wall shear stress is in the range from 0 Pa (Pascal) to about 50.0 Pa.

Cannula outer diameter is 21 Fr.

29. Results—Steady-State Wall Shear Stress
Cannulas with smaller diameters show bigger areas of high wall shear stress
Pathological values of wall shear stress over 18 Pa (see reference [8] as mentioned in section 44. below) are reached in all cases
In some cases, the area of the pathological wall shear stress exceeded the simulated geometry of the cannula. Therefore, the following section compares the areas of wall shear stress above 50 Pa.

FIG. 50A illustrates the area of WSS (Wall Shear Stress) equal to or greater than 18 Pa (Pascal) within a Cartesian coordinate system. The relevant area is the area in a cross section along the longitudinal axis of the cannula. The horizontal x-axis of the coordinate system illustrates the outer diameter of the cannula in Fr. The vertical y-axis of the coordinate system illustrates the area of the wall shear stress equal to or above 18 Pa. The unit of the area is given in cm² (square centimeter). A range from 0 cm² to 16 cm² is illustrated. The coordinate system has a left part for a flow of 3.5 l/min and for outer cannula diameters of 21 Fr (TandemHeart® cannula), 21 Fr (ReCO₂Lung® cannula), 23 Fr (ReCO₂Lung® cannula), 25 Fr (ReCO₂Lung® cannula) and 27 Fr (ReCO₂Lung® cannula).

The right part of the coordinate system is for a flow of 5l/min and for outer cannula diameters of 21 Fr (TandemHeart® cannula), 21 Fr (ReCO₂Lung® cannula), 29 Fr (ReCO₂Lung® cannula) and 31 Fr (ReCO₂Lung® cannula).

The left column in each pair of columns relates to the end-diastole (ED) state. The right column in each pair of columns relates to the end-systole (ES) state.

For all simulated 21 Fr cannulas the area of wall shear stress over 18 Pa exceeds over the whole cannula length see FIG. 50B. The wall shear stress is in the range from 0 Pa (Pascal) to 18.0 Pa.

30. Results—Steady-State Wall Shear Stress
The TandemHeart cannula shows bigger areas of wall shear stress above 50 Pa.

The left part of FIG. 51 is the same coordinate system as the coordinate system in FIG. 50A. However, the right part of FIG. 51 illustrates a further Cartesian coordinate system which illustrates the area having a wall shear stress equal to or above 50 Pa. Again, the relevant area is the area in a cross section along the longitudinal axis of the cannula. The horizontal x-axis of the further coordinate system illustrates the outer diameter of the cannula in Fr (French). The vertical y-axis of the further coordinate system illustrates the area of the wall shear stress equal to or above 50 Pa. The unit of the area is given in cm² (square centimeter). A range from 0 cm² to 0.25 cm² is illustrated. The further coordinate system is for a flow of 3.5 l/min and for outer cannula diameters of 21 Fr (TandemHeart® cannula), 21 Fr (ReCO₂Lung® cannula) and 23 Fr (ReCO₂Lung® cannula).

Also within the further coordinate system, the left column in each pair of columns relates to the end-diastole (ED) state. The right column in each pair of columns relates to the end-systole (ES) state.

31. Results—Steady-State Wall Shear Stress
Cannulas with smaller diameters show higher maximum wall shear stress values.
The maximum wall shear stress values are higher in the end-systolic configuration in all cases.
The TandemHeart cannula shows lower maximum wall shear stress at both flow rates compared to the ReCO2lung cannula with the same diameter.

FIG. 52 illustrates the maximum WSS (Wall Shear Stress) in Pa (Pascal) within a Cartesian coordinate system. The horizontal x-axis of the coordinate system illustrates the outer diameter of the cannula in Fr. The vertical y-axis of the coordinate system illustrates the maximum wall shear stress in Pa. A range from 0 Pa to 180 Pa is illustrated. The coordinate system has a left part for a flow of 3.5 l/min and for outer cannula diameters of 21 Fr (TandemHeart® cannula), 21 Fr (ReCO₂Lung® cannula), 23 Fr (ReCO₂Lung® cannula), 25 Fr (ReCO₂Lung® cannula) and 27 Fr (ReCO₂Lung® cannula).

The right part of the coordinate system is for a flow of 5l/min and for outer cannula diameters of 21 Fr (TandemHeart® cannula), 21 Fr (ReCO₂Lung® cannula), 29 Fr (ReCO₂Lung® cannula) and 31 Fr (ReCO₂Lung® cannula).

The left column in each pair of columns relates to the end-diastole (ED) state. The right column in each pair of columns relates to the end-systole (ES) state.

32. Discussion—Wall Shear Stress
Wall shear stress is directly connected to the fluid velocity. The same volume flow will cause a higher velocity in a cannula with a small diameter, therefore leading to higher wall shear stress values.
The small side holes of the TandemHeart® cannula induce flow acceleration and thereby lead to greater areas of high wall shear stress in the inflow region than observed in the ReCO2Lung® cannula.
Due to the side holes, flow enters the TandemHeart® cannula gradually (sections 12. to 14.). This favors lower maximum velocities at the edges of the cannula openings (see also section 19.), thus leading to a smaller maximum wall shear stress.
Shear-induced platelet activation may occur in all cases.
Steady-state simulations only provide data for the selected time point and furthermore disregard the influence of the preceding flow pattern. Therefore, it is possible that higher wall shear stress values and different distributions occur at other points during the cardiac cycle.

F. Pressure Distribution and Pressure Loss:

33. Results—End-Systolic Steady-State Pressure Distribution
High pressure gradient in cannula inflow tract
End-systolic configuration

ReCO₂Lung®, 3.5 l/min: FIGS. 53A to 53D, respectively 21 Fr, 23 Fr, 25 Fr and 27 Fr

ReCO₂Lung®, 5 l/min: FIGS. 53E and 53F, respectively 29 Fr and 31 Fr

TandemHeart®: FIGS. 53G and 53H, each 21 Fr but FIG. 53G for 3.5 l/min and FIG. 53H for 5l/min

The pressure is in the range from −10 mm (millimeter) Hg (mercury column) to 10.0 (millimeter) Hg (mercury column).

34. Results—End-Diastolic Steady-State Pressure Distribution
High pressure gradient in cannula inflow tract
End-diastolic configuration

ReCO₂Lung®, 3.5 l/min: FIGS. 54A to 54D, respectively 21 Fr, 23 Fr, 25 Fr and 27 Fr

ReCO₂Lung®, 5 l/min: FIGS. 54E and 54F, respectively 29 Fr and 31 Fr

TandemHeart®: FIGS. 54G and 54H, each 21 Fr but FIG. 54G for 3.5l/min and FIG. 54H for 5l/min

The pressure is in the range from −10 mm (millimeter) Hg (mercury column) to 10.0 mm Hg.

35. Results—Steady-State Pressure Distribution at the Cannula Tip
Apparent flow separation at cannula tip (high pressure gradient)

ReCO₂Lung®, 21 Fr, 3.5l/min: FIGS. 55A and 55C

TandemHeart®, 21 Fr, 3.5l/min: FIGS. 55B and 55D

End-systolic: FIGS. 55A and 55B

End-diastolic: FIGS. 55C and 55D

The pressure is in the range from −30 mm (millimeter) Hg (mercury column) to 10.0 mm Hg.

36. Results—Steady-State Pressure Distribution at the Cannula Tip
Recirculation area in TandemHeart® cannula
End-systolic configuration

ReCO₂Lung®, 21 Fr, 3.5l/min: FIG. 56A, upper part and FIG. 56A, lower part (magnification of rectangular part of FIG. 56A, upper part)

TandemHeart®, 21 Fr, 3.5 l/min: FIG. 56B, upper part and FIG. 56B, lower part (magnification of rectangular part of FIG. 56B, upper part)

The pressure is in the range from −30 mm (millimeter) Hg (mercury column) to 10.0 mm Hg for both FIGS. 56A and 56B.

37. Discussion—Steady-State Pressure Distribution at the Cannula Tip
Flow separation is highly dependent on the shape of the in-/outlet.
The shape of the TandemHeart® cannula was estimated based on the information in a patent application, see reference [1]. This does not necessarily mean that it is the exact same geometry as currently sold on the market.
Due to the side-holes of the TandemHeart® cannula, the pressure gradient is very inhomogeneous throughout the cannula cross section (see FIG. 57A). This leads to recirculation areas in the inflow region (see FIGS. 57B and 57C).

FIG. 57A illustrates the pressure distribution in the TandemHeartR cannula in a transversal cross section through holes with hole numbers 3 and 4, see section 12. The flow is 3.5 l/min and the outer diameter is 21 Fr. The inner diameter of the illustrated cannula is about 4 mm.

FIG. 57B illustrates the flow pattern in the same cross section as shown in FIG. 57A and for the same parameters. FIG. 57C illustrates the pressure distribution and the flow pattern in the TandemHeart® cannula in a longitudinal cross section at the holes with hole numbers 3 and 4, see section 12. Again the same parameters are valid as stated above for FIG. 57A.

The pressure is in the range from −30 mm (millimeter) Hg (mercury column) to 10.0 mm Hg for all three FIGS. 57A to 57C.

38. Results—Pressure Loss
Very high pressure loss when using the TandemHeart® cannula at high flows
Pressure loss decreases with increasing cannula diameter

FIG. 58 illustrates the pressure loss (delta pressure, e.g. pressure difference) over the proximal part of the cannula in mm Hg (millimeter mercury column) within a Cartesian coordinate system. The horizontal x-axis of the coordinate system illustrates the outer diameter of the cannula in Fr. The vertical y-axis of the coordinate system illustrates the pressure loss in mm Hg. A range from 0 mm Hg to 90 mm Hg is illustrated. The coordinate system has a left part for a flow of 3.5 l/min and for outer cannula diameters of 21 Fr (TandemHeart® cannula), 21 Fr (ReCO₂Lung® cannula), 23 Fr (ReCO₂Lung® cannula), 25 Fr (ReCO₂Lung® cannula) and 27 Fr (ReCO₂Lung® cannula).

The right part of the coordinate system is for a flow of 5l/min and for outer cannula diameters of 21 Fr (TandemHeart® cannula), 21 Fr (ReCO₂Lung® cannula), 29 Fr (ReCO₂Lung® cannula) and 31 Fr (ReCO₂Lung® cannula).

The left column in each pair of columns relates to the end-diastole (ED) state. The right column in each pair of columns relates to the end-systole (ES) state.

39. Discussion—Pressure Loss
Complex flow patterns at the inflow region of the TandemHeart® cannula as well as increased tip length lead to higher pressure losses.
A bigger cannula diameter is clearly beneficial regarding the pressure loss over the cannula. The pressure loss variation between different diameters will be even more significant when taking into account the full cannula length.
Pressure loss over the full length of the cannula may need to be investigated separately. The main influencing factors may be cannula length and diameter as well as bending of the cannula.

G. Left Atrial Appendix Flow:

40. Results—Left Atrial Appendage Steady-State Flow Field
At end-systole more flow in left atrial appendage with ReCO₂Lung® cannula

ReCO₂Lung®, 3.5 l/min: FIGS. 59A to 59D, respectively 21 Fr, 23 Fr, 25 Fr and 27 Fr

ReCO₂Lung®, 5 l/min: FIGS. 59E and 59F, respectively 29 Fr and 31 Fr

TandemHeart®: FIGS. 59G and 59H, each 21 Fr but FIG. 59G for 3.5 l/min and FIG. 59H for 5l/min

41. Results—Left Atrial Appendage Steady-State Flow Field
At end-diastole low flow in left atrial appendage in most cases

ReCO₂Lung®, 3.5 l/min: FIGS. 60A to 60D, respectively 21 Fr, 23 Fr, 25 Fr and 27 Fr

ReCO₂Lung®, 5 l/min: FIGS. 60E and 60F, respectively 29 Fr and 31 Fr

TandemHeart®: FIGS. 60G and 60H, each 21 Fr but FIG. 60G for 3.5 l/min and FIG. 60H for 5l/min

42. Results—Left Atrial Appendage Steady-State Flow Field
Washout of the left atrial appendage is highly dependent on the transient effects caused by the movement of the atrial wall and can hence not be simulated adequately in a steady-state simulation.
The anatomy of the left atrial appendage varies significantly from patient to patient. Therefore the effect of a trans-septal cannula on the flow in the left atrial appendage will vary as well from patient to patient.

### IV. OVERALL CONCLUSION

43. Conclusion
The ReCO₂Lung® cannula may represent an interesting alternative to the TandemHeart® ProtekSolo® cannula. The benefits are especially prominent when comparing the larger ReCO₂Lung® cannulas to the TandemHeart® cannula.
Cannulas with small diameter may show high pressure losses at high flow rates and may induce elevated wall shear stresses.
   Cannulas with larger lumen could be beneficial for this application. A limiting factor may be the patient's vessel diameter.
   Providing various cannula sizes may enable the clinician to select the largest lumen applicable with regard to the patient's anatomy.
Collapse of the atrium, suction events and repositioning of the cannula may be needed to be prevented, especially because the ReCO₂Lung® cannula only provides one inflow opening. This is where the cage has its application field.
   Fixation of the cannula tip and stabilization of the atrium may be needed and/or advantageous for the ReCO₂Lung® cannula. Again, this is where the cage has its application field.

### V. REFERENCES (WHICH ARE INCLUDED BY REFERENCE HEREWITH INTO THE PRESENT APPLICATION FOR ALL PURPOSES)

[1] Smith, D. E. et al. (2009) System for heart assist, cannula and method. US patent US 2011 0160517 A1.
[2] Badano L P, Miglioranza M H, Mih\check{a}il\check{a} S, Peluso D, Xhaxho J, Marra M P, Cucchini U, Soriani N, Iliceto S, Muraru D. (2016), Left atrial volumes and function by three-dimensional echocardiography: reference values, accuracy, reproducibility, and comparison with two-dimensional echocardiographic measurements. Circ Cardiovasc Imaging.; 9:e004229. doi: 10.1161/CIRCIMAGING.115.004229.
[3] Kou, S., Caballero, L., Dulgheru, R., Voilliot, D., De Sousa, C., Kacharava, G., . . . Lancellotti, P. (2014) Echocardiographic reference ranges for normal cardiac chamber size: results from the NORRE study. European heart journal cardiovascular Imaging, 15(6), 680-690. doi: 10.1093/ehjci/jet284.
[4] Dereli, S., Bayramo\check{g}lu, A., Özer, N. et al. (2019) Evaluation of left atrial volume and function by real time three-dimensional echocardiography in anemic patients without overt heart disease before and after anemia correction. Int J Cardiovasc Imaging, 35: 1619. doi: 10.1007/s10554-019-01609-6.
[5] Kojima, T., Kawasaki, M., Tanaka, R. et al. (2011) Left atrial global and regional function in patients with paroxysmal atrial fibrillation has already been impaired before enlargement of left atrium: Velocity vector imaging echocardiography study. European heart journal cardiovascular Imaging. 13. 227-34. 10.1093/ejechocard/jer281.
[6] Garcia, J., Sheitt, H., Bristow, M. S., Lydell, C., Howarth, A. G., Heydari, B., Prato, F. S., Drangova, M., Thornhill, R. E., Nery, P., Wilton, S. B., Skanes, A. and White, J. A. (2019) Left atrial vortex size and velocity distributions by 4D flow MRI in patients with paroxysmal atrial fibrillation: Associations with age and CHA₂DS₂-VASc risk score. J Magn Reson Imaging. doi: 10.1002/jmri.26876.
[7] Fyrenius A, Wigström L, Ebbers T, et al. (2001) Three dimensional flow in the human left atrium. Heart 2001; 86:448-455. doi: 10.1136/heart.86.4.448.
[8] Casa, L., Deaton, D., Ku, D. (2015). Role of high shear rate in thrombosis. Journal of vascular surgery. 61. doi: 10.1016/j.jvs.2014.12.050.

FIG. 61 corresponds to FIG. 1 illustrating an extra corporeal blood flow circuitry comprising two single lumen cannulas but without a cannula tip with side holes within cage arrangement 116.

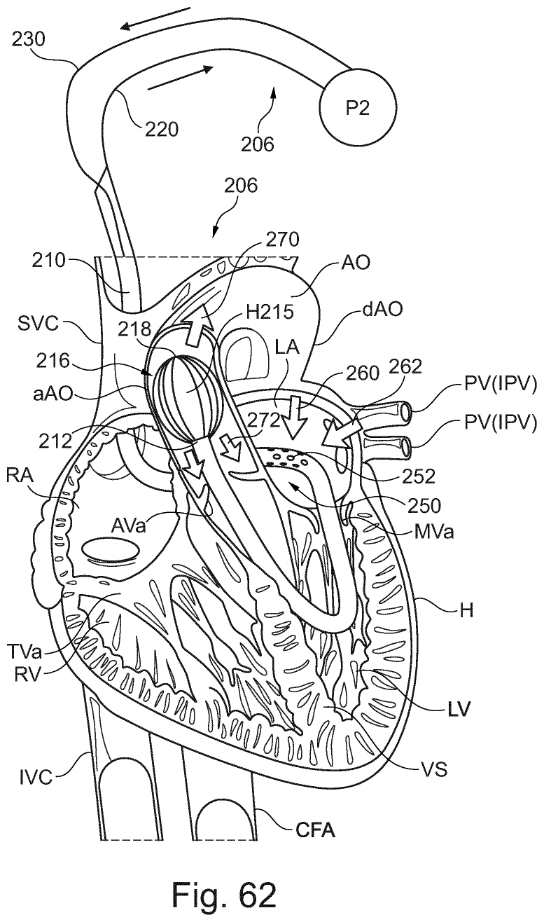

FIG. 62 corresponds to FIG. 2 illustrating an extra corporeal blood flow circuitry comprising a dual lumen cannula but without a cannula tip with side holes within cage arrangement 216.

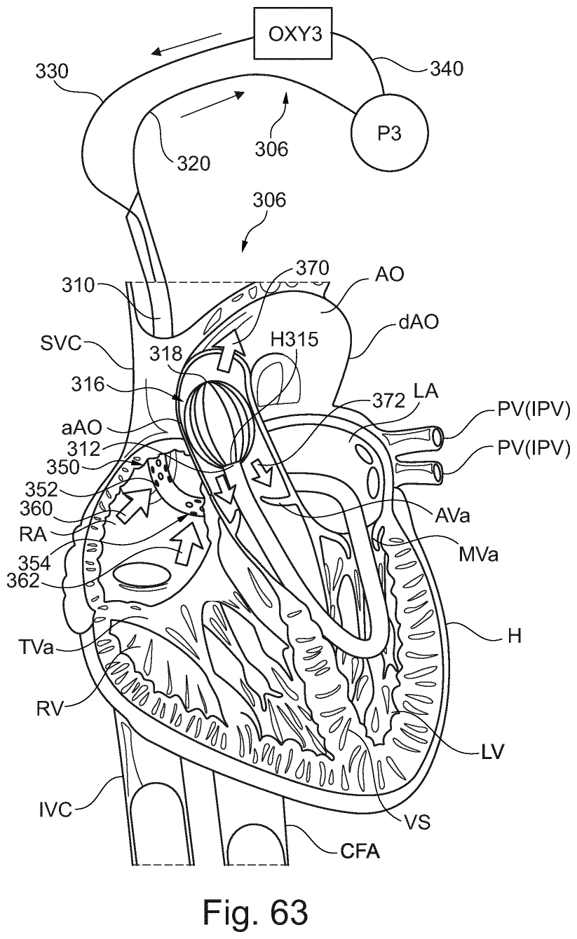

FIG. 63 corresponds to FIG. 3 illustrating an extra corporeal blood flow circuitry comprising a dual lumen cannula, a blood pump and oxygenator but without a cannula tip with side holes within cage arrangement 316.

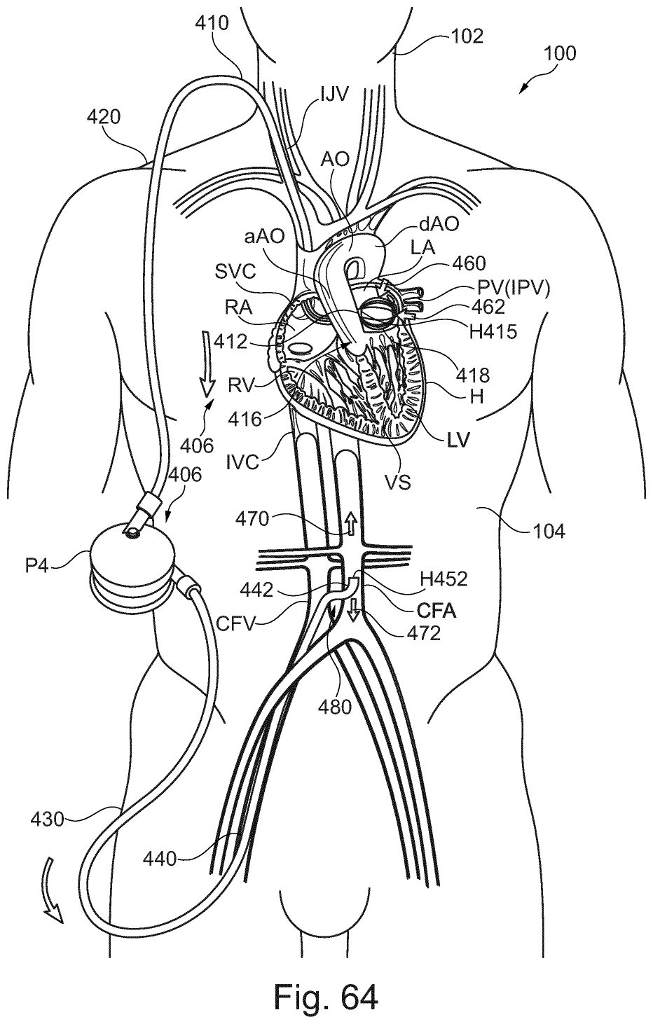

FIG. 64 corresponds to FIG. 4 illustrating a transcaval extra corporeal blood flow circuitry comprising two single lumen cannulas but without a cannula tip with side holes within the cage arrangement 416.

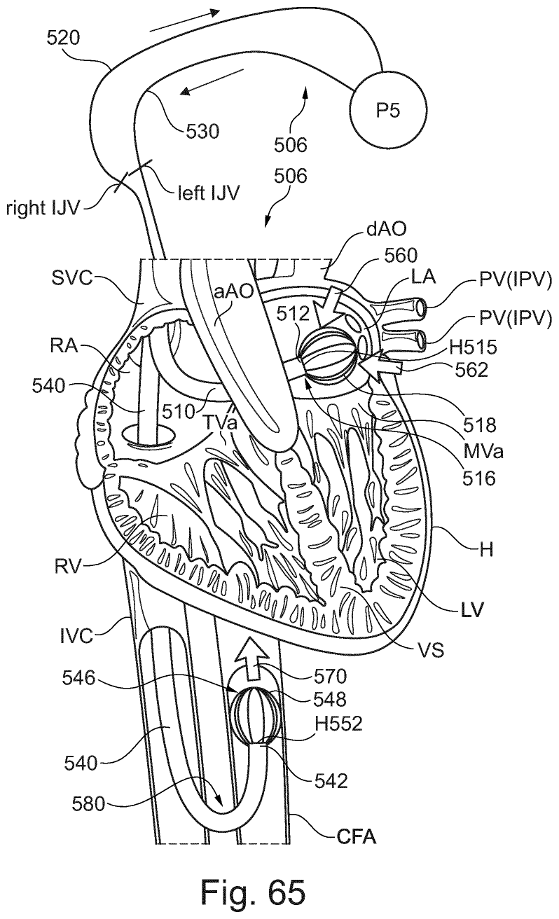

FIG. 65 corresponds to FIG. 5 illustrating a transcaval extra corporeal blood flow circuitry comprising two single lumen cannulas both inserted through jugular veins but without a cannula tip with side holes within cage arrangements 516, 546.

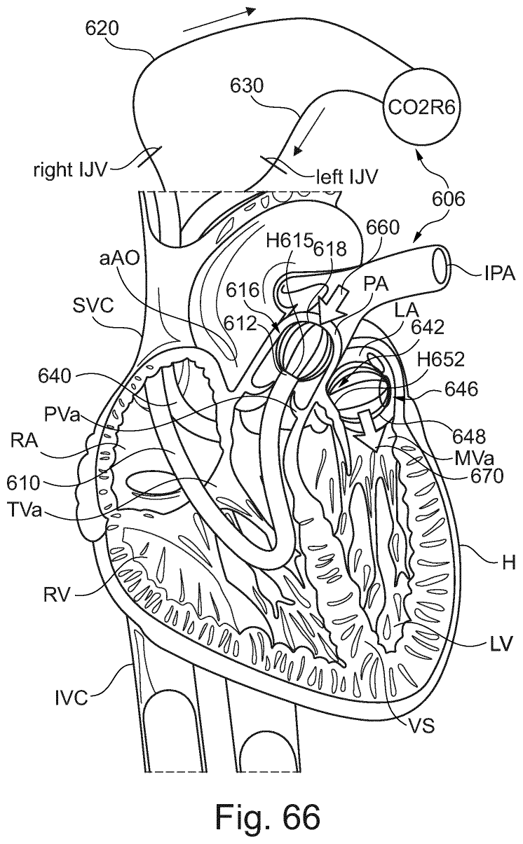

FIG. 66 corresponds to FIG. 6 illustrating an extra corporeal lung assist blood flow circuitry without pump comprising two single lumen cannulas and a carbon dioxide removal device but without a cannula tip with side holes within cage arrangements 616, 646.

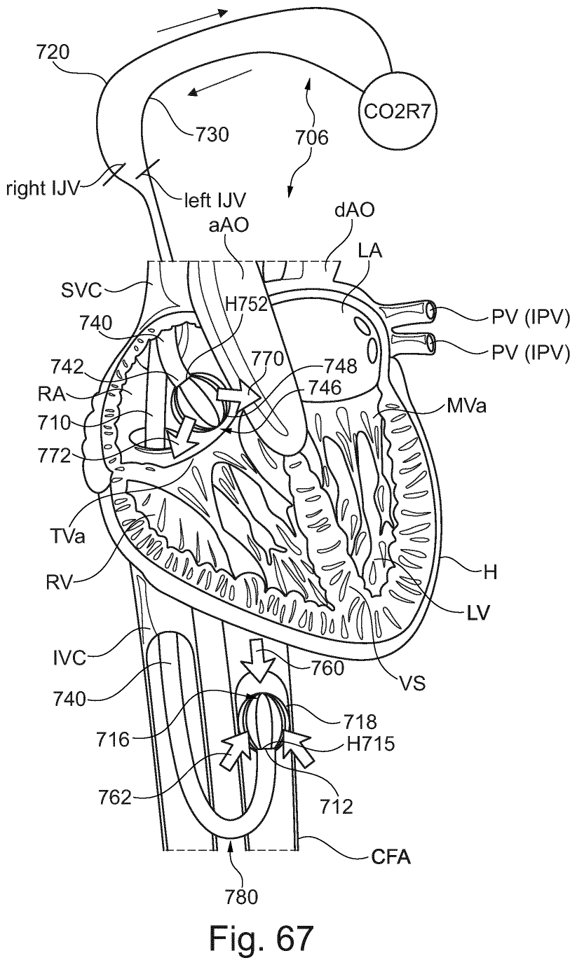

FIG. 67 corresponds to FIG. 7 illustrating a transcaval extra corporeal lung assist blood flow circuitry without pump comprising two single lumen cannulas and a carbon dioxide removal device but without a cannula tip with side holes within cage arrangements 716, 746.

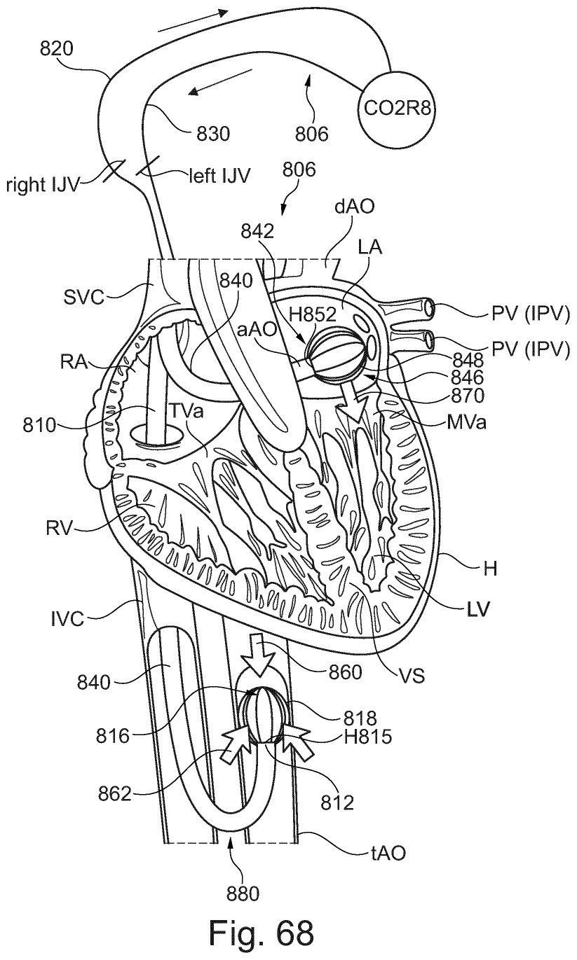

FIG. 68 corresponds to FIG. 8 illustrating a transcaval transseptal extra corporeal lung assist blood flow circuitry without pump comprising two single lumen cannulas and a carbon dioxide removal device but without a cannula tip with side holes within cage arrangements 816, 846.

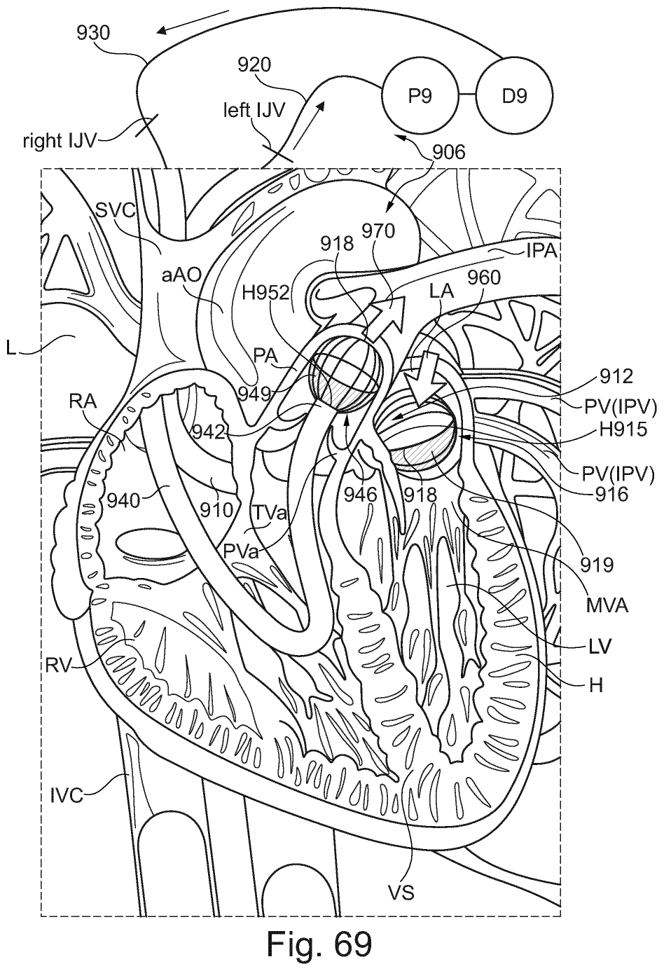

FIG. 69 corresponds to FIG. 9 illustrating an extra corporeal circular lung perfusion blood flow circuitry comprising two single lumen cannulas, a pump and a further device but without a cannula tip with side holes within cage arrangements 916, 946.

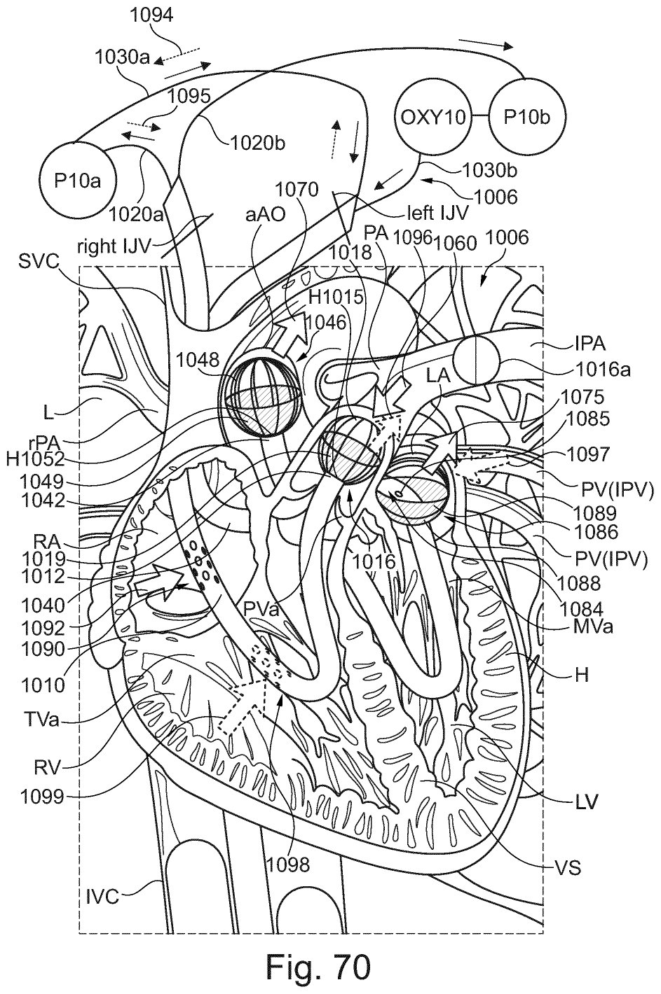

FIG. 70 corresponds to FIG. 10 illustrating an extra corporeal retrograde lung perfusion circular blood flow circuitry comprising two dual lumen cannulas, an alternative embodiment with antegrade lung perfusion, an alternative embodiment with lobe dedicated lung perfusion and an embodiment for right ventricle assist but without a cannula tip with side holes within cage arrangements 1016, 1046.

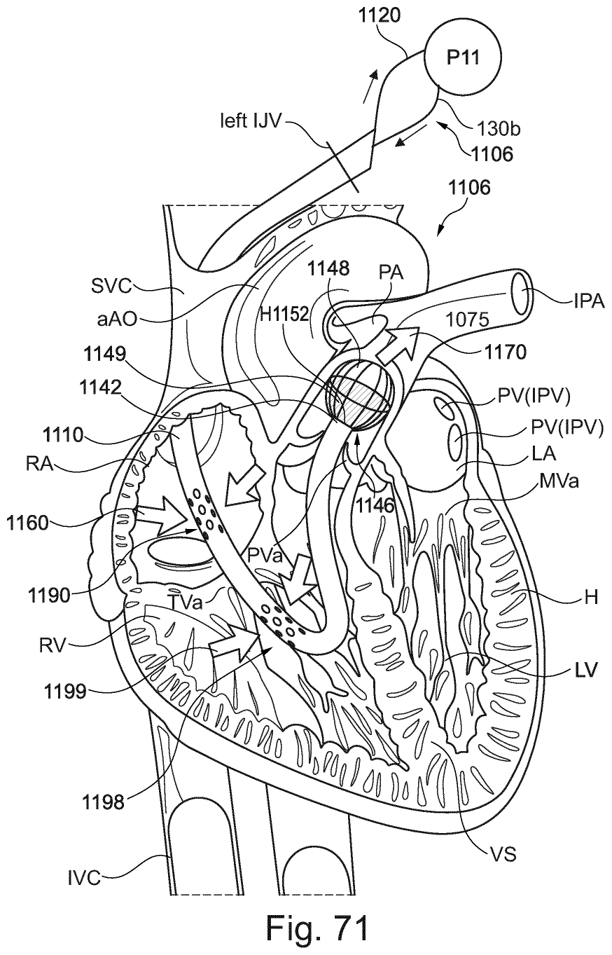

FIG. 71 corresponds to FIG. 11 illustrating a right ventricle assist circuitry with one inlet stage or with multi inlet stages but without a cannula tip with side holes within cage arrangement 1146.

FIG. 72 corresponds to FIG. 15 illustrating an embodiment of a dual lumen system comprising at least one pre-bended cannula but without a cannula tip with side holes within cage arrangement DVA2.

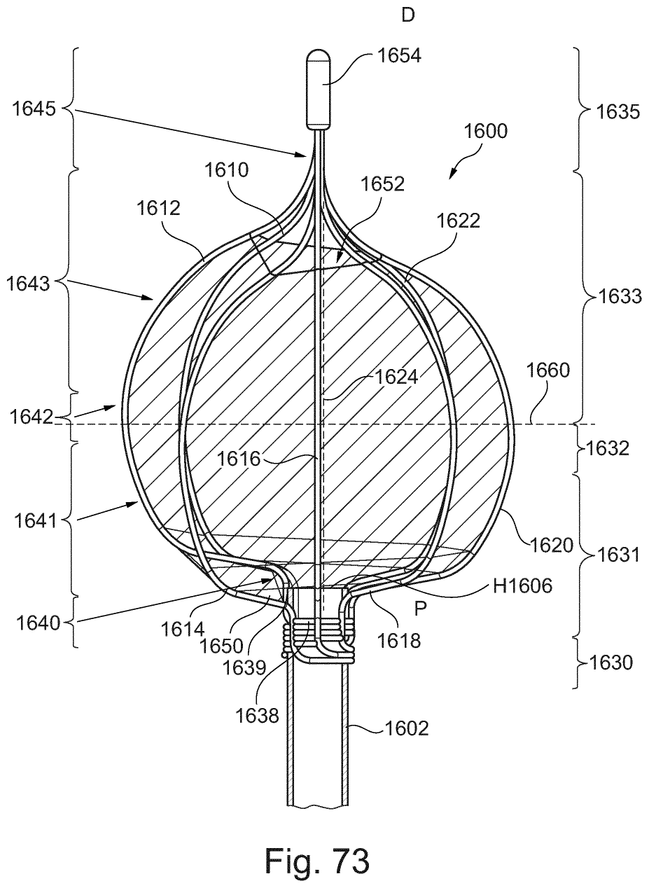

FIG. 73 corresponds to FIG. 16 illustrating a cage arrangement comprising a membrane having an opening that faces distally but without a cannula tip with side holes within cage arrangement 1600.

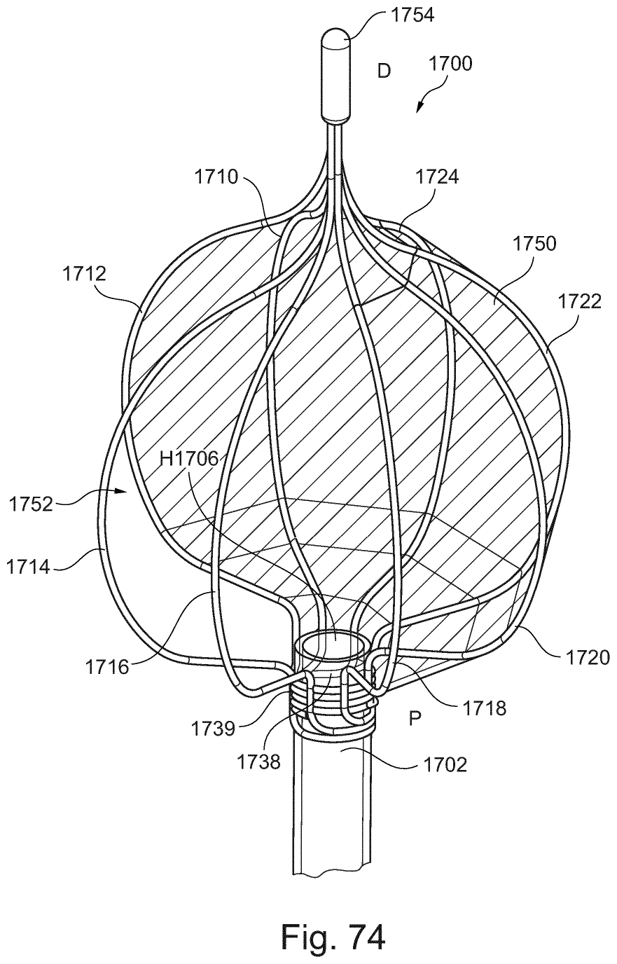

FIG. 74 corresponds to FIG. 17 illustrating a cage arrangement comprising a membrane having an opening that faces laterally but without a cannula tip with side holes within cage arrangement 1700.

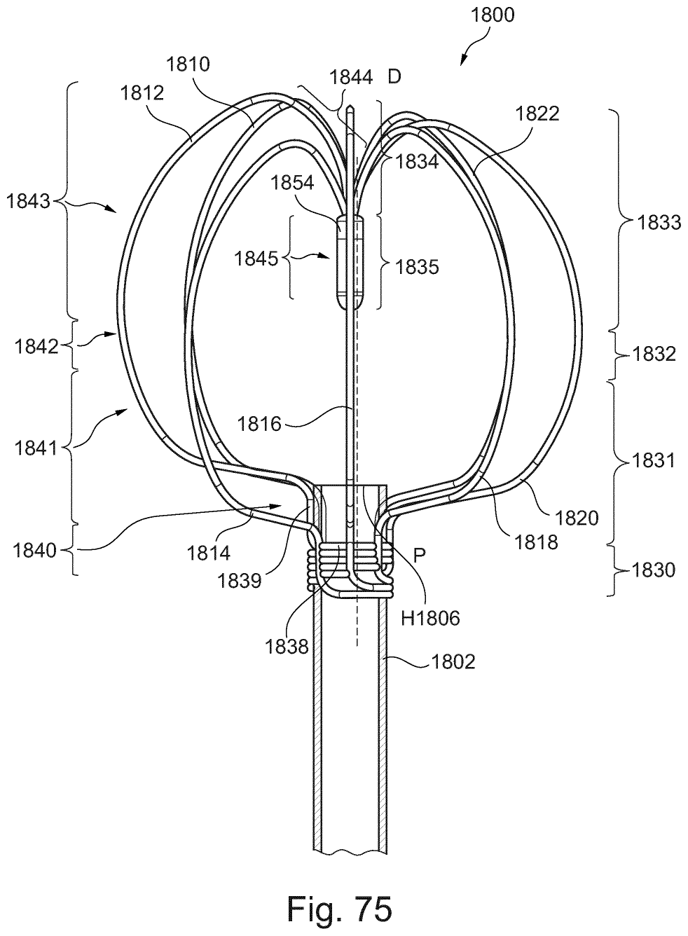

FIG. 75 corresponds to FIG. 18 illustrating a cage arrangement comprising a portion that is bended backwards but without a cannula tip with side holes within cage arrangement 1800.

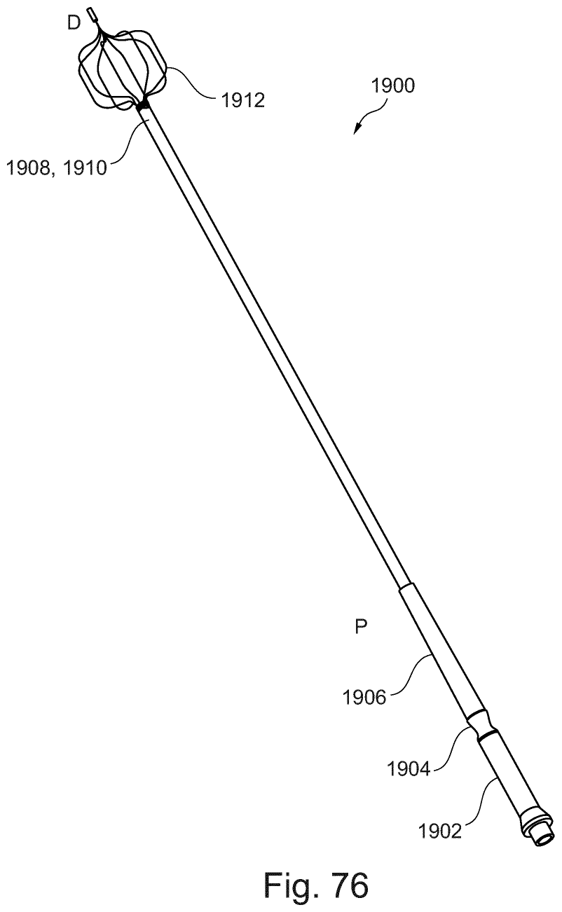

FIG. 76 corresponds to FIG. 19 illustrating a cannula comprising a cage arrangement having wires that are arranged in parallel with regard to each other but without a cannula tip with side holes within cage arrangement 1900.

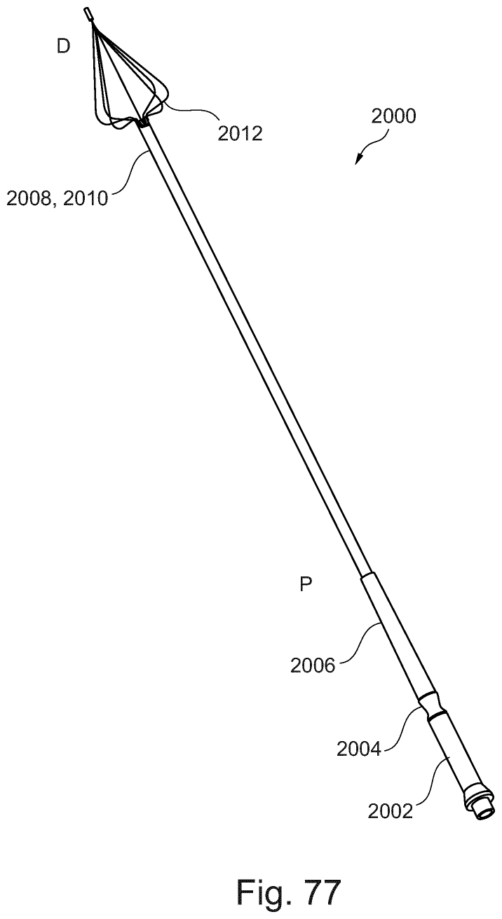

FIG. 77 corresponds to FIG. 20 illustrating a cannula comprising a cage arrangement having a cone like shape but without a cannula tip with side holes within cage arrangement.

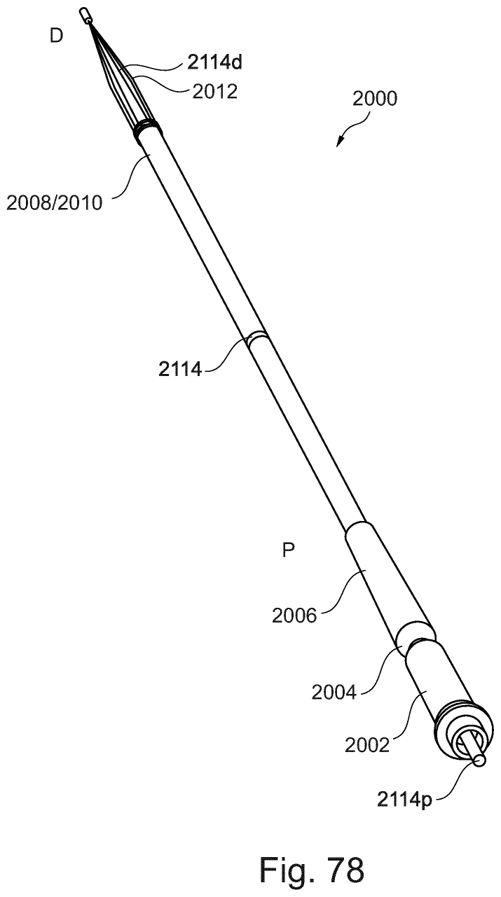

FIG. 78 corresponds to FIG. 21 illustrating the cannula of FIG. 77 in a state in which an introducer stretches the cage arrangement for introducing the cannula into a body but without a cannula tip with side holes within cage arrangement.

In all embodiments one of the following methods may be used to bring or guide a guide wire and/or a catheter around or along the acute angle within the left ventricle LV. At least one snare may be used to catch the catheter and/or the guide wire in the left ventricle LV. The methods may be performed independent whether there is jugular access or a femoral access or another access for the catheter and/or the guide wire.

Variant A (catching the catheter with the snare):

1) Introducing a catheter through the right atrium RA, the atrial septum AS (a puncturing step may be performed earlier or using the catheter, e.g. using a needle and/or RF (radio frequency) tip/wire within the catheter). The catheter may be introduced further through the hole (puncture) in the atrial septum AS through left atrium LA, through mitral valve MV into the left ventricle LV.

2) Introducing a snare from descending aorta AO through aortic valve AV into left ventricle LV. This step may be performed also before step 1.

3) Catching the catheter in the left ventricle LV using the snare.

4) Pulling the snare and the distal end of the catheter therewith to the aorta AO.

5) Introducing a guide wire through the catheter.

6) Forwarding the guide wire out of the distal end of the catheter. Slight loosening of the snare may be optionally performed thereby.

7) As the guide wire is already within the snare, pull back the snare to a region in which only the guide wire is located but not the catheter.

8) Fix the guide wire using the snare, e.g. contract the snare and/or tighten the snare.

9) Optional, externalizing for instance the distal end of the guide wire out of the body. This step is optionally, because the proximal end of the snare is already outside of the body.

10) Remove catheter, e.g. pull back the catheter.

11) Introduce cannula using the guide wire, e g pushing the cannula along and/or over the guide wire until it is on its final place.

Variant B (catching the guide wire with the snare):

1) Introducing a catheter through the right atrium RA, through the atrial septum AS (a puncturing step may be performed earlier or through catheter, use needle and/or RF (radio frequency) tip/wire). Introducing the catheter further through left atrium LA, mitral valve MV into the left ventricle LV.

2) Introducing a guide wire through the catheter until the distal end of the guide wire comes out of the distal end of the catheter within the left ventricle LV. The RF wire may be used also as a guide wire.

3) Introducing a snare from descending aorta AO through aortic valve AV into left ventricle LV. This step may be performed before step 1 and/or before step 2.

3) Catching the distal end of the guide wire in the left ventricle LV using the snare.

4) Fixation of the guide wire using the snare.

5) Pulling the snare and the distal end of the guide wire therewith to the aorta AO.

6) Optional, externalizing guide wire by pulling it out of the body using the snare. This step is optional as the snare is already outside of the body from where it has been introduced.

7) Remove catheter, e.g. by pulling it back along the guide wire.

8) Introduce cannula over/along the guide wire until it is on place.

The following method may also be used in all corresponding embodiments for introducing a cannula jugularly transseptally:

1) Introduce a first snare into an internal jugular vein IJV, for instance into the right jugular vein RJV or into the left jugular vein LJV.

2) Advancing the first snare to inferior vena cava IVC.

3) Introducing a catheter into a common femoral vein CFV (left or right).

4) Advancing the catheter through the first snare into an inferior vena cava IVC.

5) Advancing the catheter through the first snare into the vena cava VC in an antegrade fashion.

6) Advancing the catheter through the first snare into the right atrium RA in an antegrade fashion.

7) Advancing the catheter through the first snare and from the right atrium RA transseptally through the atrial septum into the left atrium LA in an antegrade fashion. Puncturing of atrial septum may have been performed earlier. Alternatively, the catheter is used to puncture the atrial septum, for instance using a needle or using a RF (radio frequency) wire/tip which is introduced trough the catheter.

8) Advancing the catheter through the first snare and advancing the catheter across the mitral valve MV and into the left ventricular outflow tract, e.g. the left ventricle LV.

9) Advancing a second snare in the ascending aorta AO catching and snaring a distal portion of the catheter (Variant A) within the left ventricle LV. The second snare may optionally be introduced through an artery, which may include, but is not limited to, a radial artery, a brachial artery, an axillary artery, a subclavian artery, a carotid artery, or common femoral artery, and advanced retrograde into the aorta AO and into the left ventricle LV. The second snare may be already introduced before the catheter is introduced. Alternatively, a guide wire may be inserted into the catheter until a distal end of the guide wire comes out of a distal opening of the catheter. This distal end of the guide wire is then caught and snared within the left ventricle (Variant B)

10) Pulling the catheter (Variant A) or the guide wire (Variant B) into the aorta AO in an antegrade fashion using the second snare.

11) In variant A, advancing a guide wire through the catheter and through the first snare in antegrade fashion to the ascending aorta AO and through the second snare. Snaring the distal end of the guide wire in variant A but not the catheter.

12) In both variants A and B remove the catheter with the guide wire remaining in the heart H and through the first snare after the catheter is removed.

13) Externalizing a proximal portion of the guide wire from femoral vein, through inferior vena cava IVC, through inferior vena cava SVC, into the internal jugular vein IJV and then out of the internal jugular vein IJV using the first snare, for instance left jugular vein LJV or right jugular vein RJV. In some embodiments the snare may externalize a different portion of the guide wire, for instance an intermediate portion.

14) Advancing a cannula using the guide wire and/or along and/or over the guide wire from the internal jugular vein IJV. The cannula may be any of the cannulas described in this specification or known in the art. Especially, an outer cannula may be advanced over the guide wire from the internal jugular vein IJV. An inner cannula may optionally be advanced through a port proximal of the distal end of the outer cannula. The inner cannula and the outer cannula may be positioned as described in this description, or if a single multi-lumen cannula is used, it may be positioned in a similar manner

15) Optionally, a distal portion of the guide wire may be externalized out of the body through the artery. This step is optional because the second snare is already externalized and may form a secure anchor for the distal portion of the guide wire.

Subclavian arteries/veins or other arteries/veins may be used for introducing the snare(s) because the snares require smaller diameters, e.g. less than 10 French (1 French equal to ⅓ mm (millimeter)) or less than 8 French, e.g. more than 3 French, compared to the diameters of the cannula(s).

In the following details of a method for puncturing transseptally through the atrial septum AS of the heart H are provided. However, other methods may be used as well, for instance using a needle. A catheter and/or a wire may be used which has a distal tip which can be heated, for instance using RF (radio frequency) energy, alternating current (ac), direct current (dc) etc. Thus, e.g. a hole may be burned into the septum, e.g. the atrial septum AS, during puncturing, for instance using temperatures above 100° C. (degrees Celsius) or above 200° C., less than 1000° C. for instance.

The RF (radio frequency) may be in the range of 100 kHz (kilohertz) to 1 MHz (Megahertz) or in the range of 300 kHz to 600 kHz, for instance around 500 MHz, i.e. in the range of 450 kHz to 550 kHz, e.g. 468 kHz.

The power of the radio frequency energy may have a maximum of 50 Watt. A power range of 5 W (watt) to 100 W may be used, for instance a range of 10 W to 50 W.

A sinus current/voltage may be used for the RF. The sinus current/voltage may be continuous. Alternatively, a pulsed sinus current/voltage may be used for the RF.

All parameters or some of the parameters of the RF equipment may be adjustable by an operator who performs the puncturing, for instance dependent on the specifics of the septum, e.g. normal septum, fibrotic septum, aneurysmal septum, etc. Preferably, the power may be adjustable.

A solution of Baylis Medical (may be a trademark), Montreal, Canada may be used, for instance NRG® trans-septal needle or Supra Cross® RF Wire technology. RF generator of type RFP-100A or a further development of this model may be used. This RF generator uses for example a frequency of 468 kHz (kilohertz).

A single puncture of the septum may be performed from a jugular access or from a femoral access or from another appropriate access using the RF energy. Smaller angles may be possible for the catheter if for instance compared with a needle.

Alternatively, the RF method may be used also if two separate punctures are made in the septum. However, usage of needles is possible as well. One of the punctures using the RF method may be made through left jugular vein LJV and the other puncture of the atrial septum AS may be made through the right jugular vein RJV.

It is possible to introduce both guide wires first through the atrial septum AS. Preferably, separate holes are used for each of the guide wires. Guide wire(s) may be used which include an RF tip. Alternatively, the wire(s) having the RF tip may be pulled back and a further wire may be introduced through the catheter.

Only after both guide wires are in place, both cannulas may be introduced using a respective one of the guide wires.

Alternatively, the first puncture may be performed using RF energy or a needle. Thereafter, the first cannula for blood transfer is inserted using the first guide wire. After insertion of the first cannula, the second puncture may be made. A second guide wire or the first guide wire may be used to introduce the second cannula.

Puncturing of the atrial septum may be assisted by at least one medical imaging method, preferably by at least two medical imaging methods.

US (ultra-sonic) echo imaging may be used to visualize the movement of heart H and the location of the valves of heart H. No dangerous radiation may result from ultra-sonic imaging. An ultra-sonic transmitter may be introduced for instance via the esophagus, e.g. trans esophagus echo (TEE) may be used.

X-ray radiation preferably in combination with fluorescence (fluoroscopy), may be used in order to visualize the location of catheters (comprising for instance at least one X-ray marker, or the devises are usually radiopaque) and/or the location of guide wire(s), snares etc.

Thus, transseptal puncturing or puncturing of other tissue may be guided by TEE and by fluoroscopy or by other imaging methods. At least two different image generating methods may be used.

In all embodiments mentioned above, it is also possible to use a soft guide wire and a stiffer guide wire which does not bend so easy if compared with the soft guide wire. The following steps may be performed, preferably in combination with snaring:

1) Introduce a soft guide wire.

2) Introduce catheter using the soft wire as a guide.

3) Optionally, remove soft wire, for instance by pulling back the soft wire out of the catheter.

4) Introduce stiffer guide wire into the catheter, e.g. there may be a change of wire from soft wire to the stiffer wire.

The catheter may be removed, e.g. pulled back. Thereafter, the stiffer wire may be used to introduce a cannula or cannulas.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. Further, it is possible to combine embodiments mentioned in the first part of the description with examples of the second part of the description which relates to FIGS. 1 to 78. Moreover, it is possible to combine embodiments mentioned in the first part of the Figures, i.e. FIGS. 1 to 11, with examples of the second part of the Figures, which relates to FIGS. 12 to 27.

1. Cannula (110, O1 to O3, I1 to I3) comprising:
a lumen portion (LP) that extends axially between a proximal part of the cannula (110, O1 to O3, I1 to I3) and at least one distal part of the cannula (110, O1 to O3, I1 to I3), and
an expandable arrangement at the at least one distal part of the lumen portion,
wherein the expandable arrangement is adapted to have an expanded state and a non-expanded state,
wherein in the expanded state a volume defined by the expandable arrangement is greater than the volume defined by the expandable arrangement in the non-expanded state.

2. Cannula (110, O1 to O3, I1 to I3) according to claim 1, wherein the lumen portion (LP) is less expandable than the expandable arrangement, and/or
wherein the volume in the expanded state is at least by factor 1.5 or 2 greater than the volume in the non-expanded state.

3. Cannula (110, O1 to O3, I1 to I3) according to claim 1 or 2, wherein the lumen portion (LP) is adapted to guide an introducer member,
and wherein the expandable arrangement comprises a contact area that is adapted to have mechanical contact with the introducer member,
and wherein the expandable arrangement is configured such that it changes from the non-expanded state to the expanded state if the introducer member is moved away from the contact area.

4. Cannula (110, O1 to O3, I1 to I3) according to one of the preceding claims wherein the expandable arrangement comprises a plurality of wires (118) which are connected to the lumen portion in a connection region of the respective wire.

5. Cannula (110, O1 to O3, I1 to I3) according to claim 4,
wherein at least two wires, at least three wires or all wires of the plurality of wires (118) are connected with each other and/or with a common connection element in an end region of the respective wire remote from the connection region as seen along the extension of the respective wire.

6. Cannula (110, O1 to O3, I1 to I3) according to claim 5, wherein in the expanded state at least two wires, at least three wires or all wires of the plurality of wires extend between the connection region and the end region of the respective wire without mechanical contact to other wires of the expandable arrangement and/or without crossing other wires of the expandable arrangement and/or without being crossed by other wires of the expandable arrangement.

7. Cannula (110, O1 to O3, I1 to I3) according to one of the claims 4 to 6, wherein in the expanded state at least two wires, at least three wires or all wires of the plurality of wires (118) have the same or similar shape in the expanded state and/or before assembly of the expandable arrangement,
and/or wherein in the non-expanded state at least two wires, at least three wires or all wires of the plurality of wires (118) have the same or similar shape,
wherein preferably a sequence of angularly consecutive wires, preferably a sequence that comprises all wires of the plurality of wires, comprises the same axial offset between two angularly adjacent wires and/or the same angularly offset between two angularly adjacent wires.

8. Cannula (110, O1 to O3, I1 to I3) according to one of the claims 4 to 7, wherein at least one wire, at least two or all wires of the plurality of wires (118) has or have a portion of the respective wire (118, 1610 to 1624) that extends in the connection region of the wire angularly, preferably only angularly, along or around the outer circumference of the lumen portion.

9. Cannula (110, O1 to O3, I1 to I3) according to one of the claims 4 to 9, wherein at least one wire, at least two wires or all wires of the plurality of the wires (118, 1610 to 1624) comprise a proximal mounting portion (1640) that comprises a circumferential portion (1638) in which the wire (1612) extends along a circular or oval curve, preferably along at least two thirds or at least three quarters of the circumference of a circle or of an ellipse but not along the complete circumference or preferably along at least on winding or at least two windings, and
a straight or less bended portion (1639) that is preferably arranged outside a plane in which the circumferential portion (1638) is arranged.

10. Cannula (110, O1 to O3, I1 to I3) according to claim 9, wherein the straight portion (1639) or the less bended portion of at least one wire, of at least two wires or of all wires of the plurality of the wires (1610 to 1624) extend across at least one of the wires, at least some of the wires, preferably axially and/or across circumferential portions.

11. Cannula (110, O1 to O3, I1 to I3) according to claim 10, wherein the straight portions (1639) or the less bended portions of at least one wire, of at least two wires or of all wires of the plurality of wires (118, 1610 to 1624) are connected to the circumferential portion (1638) of at least some of the wires (1610 to 1624) by welding or soldering or by using an adhesive.

12. Cannula (110, O1 to O3, I1 to I3) according to one of claims 4 to 11, wherein the wires (118) comprise a material that has a shape memory, preferably a shape memory that is depending on temperature or that is not or slightly depending on temperature,
wherein the material of the wires (118) preferably comprises or preferably consist of Nitinol, titanium, titanium alloys or copper-aluminum-nickel alloys.

13. Cannula (110, O1 to O3, 1602, I1 to I3) according to one of the claims 4 to 12, wherein the expandable arrangement comprises a proximal portion (1631), wherein in the expanded state of the expandable arrangement the distance between angularly neighboring wires (118, 1048, 1088) in the proximal portion increases with increasing distance to a mounting portion of the wires (118, 1048, 1088) on the cannula (110, O1 to O3, 1602, I1 to I3),
and wherein the expandable arrangement comprises a distal portion (1633), wherein in the expanded state of the expandable arrangement the distance between angularly neighboring wires (118, 1048, 1088) in the distal portion decreases with increasing distance to the mounting portion of the wires (118, 1048, 1088),
and wherein the expandable arrangement comprises an optional transition portion (1632), wherein in the expanded state of the expandable arrangement the distance between angularly neighboring wires (118, 1048, 1088) in the optional transition portion is constant with increasing distance to the mounting portion of the wires (118, 1048, 1088),
wherein preferably an axial extension of the transition portion is longer than an axial extension of the proximal portion in the expanded state and/or longer than an axial extension of the distal portion in the expanded state.

14. Cannula (1802, I1 to I3) according to one of the claims 4 to 13, wherein the expandable arrangement comprises in the expanded state following a distal portion (1833):
an optional radially extending straight portion in which the wires extend only radially inward at the same axial position for at least 1 mm, at least 2 mm, at least 3 mm, at least 4 mm or at least 5 mm, a backwardly bended portion (1834), preferably an inwardly bended portion,
wherein preferably within the backwardly bended portion the wires (1810 to 1824) change direction and/or neighboring wires (1810 to 1824) have decreasing distances with decreasing distance to the mounting portions of the wires (1810 to 1824),
and preferably wherein the expandable arrangement comprises a cage tip portion (1835) following the backwardly bended portion (1834), wherein in the cage tip portion (1835) the cage wires (1810 to 1824) are connected with each other.

15. Cannula (110, O1 to O3, 1602, I1 to I3) according to one of the claims 4 to 14, wherein at least one wire, at least three wires or all wires of the plurality of wires (118) comprises in the expanded state a mounting portion (1640, 1840) that comprises:
preferably a circumferential portion (1638, 1838) in which the wire is bended circular or oval, for instance along at least three quarters of the circumference of a circle or of an ellipse but not along the complete circumference or preferably along at least on winding or at least two windings, and
preferably a straight portion (1639, 1839) or less bended portion that is preferably arranged parallel to the extended longitudinal axis of the lumen portion (LP),
a proximal portion (1641, 1841) in which the wire (1612, 1812) has an increasing radial distance to the extended longitudinal axis of the lumen portion (LP) with increasing distance to the mounting portion (1640, 1840),
an optional transition portion (1642, 1842) in which the wire (1612, 1812) has a constant radial distance to the extended longitudinal axis of the lumen portion (LP) with increasing distance to mounting portion (1640, 1840),
a distal portion (1643, 1843) in which the wire (1612, 1812) has a decreasing radial distance to the extended longitudinal axis of the lumen portion (LP) with increasing distance to mounting portion (1640, 1840), and
a backwardly bended wire portion (1844), and
preferably a radially extending straight portion in which the wire extends only radially inward at the same axial position for at least 1 mm, at least 2 mm, at least 3 mm, at least 4 mm or at least 5 mm,
preferably a cage tip portion (1645, 1845) that may be covered by plastic cap and/or in which the wire (1612, 1812) is parallel to the extended longitudinal axis of the lumen portion.

16. Cannula (110, O1 to O3, I1 to I3) according to one of the claims 4 to 15, wherein wires (118) of the expandable arrangement of the cannula (110, O1 to O3, I1 to I3) are distributed angularly such that, in a given axial position preferably in the expanded state, for a first pair of angularly neighboring wires the wires forming the pair have a first distance relative to each other and that angularly neighboring wires forming a second pair of neighboring wires have a second distance relative to each other that is greater than the first distance, preferably equal to or greater than twice the first distance.

17. Cannula (110, O1 to O3, I1 to I3) according to any one of the preceding claims, wherein the expandable arrangement comprises at least one membrane that is connected to at least two of the plurality of wires,
and wherein preferably the membrane is folded or less stretched in the non-expanded state and expanded in the expanded state of the expandable arrangement.

18. Cannula (110, O1 to O3, I1 to I3) according to claim 17, wherein in the expanded state the membrane defines an opening that faces distally relative to the longitudinal axis of the lumen portion of the cannula (O1 to O3, I1 to I3), or
wherein in its expanded state the membrane defines an opening that faces laterally relative to the cannula (O1 to O3, I1 to I3), or
wherein in its expanded state the membrane defines an opening that faces proximally relative to the longitudinal axis of the lumen portion (LP) of the cannula (O1 to O3, I1 to I3).

19. Cannula (110, O1 to O3, I1 to I3) according to one of the claims 1 to 18, wherein the cannula (110, O1 to O3, I1 to I3) is adapted to be inserted endovascular into the heart (H), and preferably further into the aorta (AO) or into the pulmonary artery (PA).

20. Cannula (110, O1 to O3, I1 to I3) according to one of the claims 1 to 19, wherein the cannula does not extend within the expandable arrangement or does maximally extend into the expandable arrangement by at most 10 mm or at most 5 mm and/or wherein the cannula comprises at least one end hole or a single end-hole through which at least 25 volume percent, at least 50 volume percent, at least 75 volume percent or at least 90 volume percent or all of the flow flows into or out of the cannula.

21. Cannula system (CS1 to CS3) comprising a cannula (110, O1 to O3, I1 to I3) according to one of the preceding claims wherein the cannula is a first cannula, and comprising:
a second cannula (I1 to I3) that is arranged at least partially within the first cannula (110, O1 to O3) and/or that is adapted to be inserted into and/or through the first cannula (110, O1 to O3) or that is adapted such that the first cannula is arranged within the second cannula or such that the first cannula can be inserted into and/or through the second cannula,
and preferably a first introducer member (2114) for introducing the first cannula (110, O1 to O3) and for stretching the expandable arrangement,
and preferably a second introducer member for introducing the second cannula (I1 to I3).

22. Cannula system (CS1 to CS3) according to claim 21, wherein the second cannula (I1 to I3) comprises a flexible further lumen portion that extends axially between a proximal part of the second cannula and a distal part of the second cannula,
wherein at least one distal part of the further lumen portion carries a further expandable arrangement,
wherein the expandable arrangement of the second cannula (I1 to I3) is adapted to have an expanded state in which a volume defined by the expandable arrangement of the second cannula (I1 to I3) is greater than the volume defined by the expandable arrangement of the second cannula (I1 to I3) in a non-expanded state.

23. Cannula system (CS1 to CS3) according to claim 21 or 22, wherein wires (118) of the expandable arrangement of the first cannula are distributed angularly such that, in a given axial position preferably in the expanded state, for a first pair of angularly neighboring wires the wires forming the pair have a first distance relative to each other and that angularly neighboring wires forming a second pair of neighboring wires have a second distance relative to each other that is greater than the first distance, preferably equal to or greater than twice the first distance, and/or
wherein preferably the second cannula is insertable or is inserted through a gap between the wires forming the second pair.

24. Cannula system (CS1 to CS3) according to claim 23, wherein the expandable arrangement of the first cannula (O1 to O3) comprises a membrane, wherein the membrane is folded or less stretched in the non-expanded state of the expandable arrangement of the first cannula (O1 to O3) and wherein the membrane is expanded in the expanded state of the expandable arrangement of the first cannula (O1 to O3),
and wherein a segment of the membrane is arranged between the wires of the expandable arrangement of the first cannula (O1 to O3), preferably between the wires of the second pair,
and wherein the segment of the membrane has an insertion opening that is adapted to the diameter of the second cannula (I1 to I3),
and wherein preferably the insertion opening is adapted such that the second cannula is insertable or is inserted through the insertion opening thereby closing at least the main part or all of the insertion opening.

25. Cannula system (CS1 to CS3) according to one of the claims 21 to 24, wherein the first cannula does not extend within the expandable arrangement of the first cannula or does maximally extend into the expandable arrangement of the first cannula by at most 10 mm or at most 5 mm or at most 3 mm and/or wherein the first cannula and/or wherein the cannula comprises at least one end hole or a single end-hole through which at least 25 volume percent, at least 50 volume percent, at least 75 volume percent or at least 90 volume percent or all of the flow flows into or out of the cannula.

26. Cannula system (CS1 to CS3) according to one of the claims 21 to 25, comprising a further cannula according to one of the claims 1 to 20, wherein the further cannula is a third cannula and comprising:
a fourth cannula (I1 to I3) that is arranged at least partially within the third cannula or that is adapted to be inserted into and/or through the third cannula (110, O1 to O3) or that is arranged within the fourth cannula or that is adapted such that the third cannula can be inserted into and/or through the fourth cannula,
wherein preferably the fourth cannula carries an expandable arrangement that is adapted to have an expanded state and a non-expanded state.

27. Method for inserting a cannula into a subject, comprising:
providing a cannula (110, O1 to O3, I1 to I3) that comprises at least one lumen portion (LP) and at least one expandable arrangement on at least one distal part of the at least one lumen portion (LP), inserting the cannula (110, O1 to O3, I1 to I3) into a body (100) of the subject whereby the at least one expandable arrangement is in a non-expanded state,
expanding the at least one expandable arrangement thereby securing the cannula within the body (100), using a lumen within the lumen portion (LP) to guide a fluid into the body (100) and/or out of the body (100).

28. Method according to claim 27, comprising:
inserting an introducer member (2114) into the cannula (110, O1 to O3, I1 to I3) in order to bring the expandable arrangement into the non-expanded state in which the expandable arrangement defines a first volume,
inserting the cannula (110, O1 to O3, I1 to I3) into the body (100) of the subject using the introducer member until the distal end (D) of the cannula (110, O1 to O3, I1 to I3) reaches a final destination place, thereafter pulling back the introducer member (2114) leaving the expandable arrangement in the expanded state that defines a second volume that is greater than the first volume, preferably at least twice the first volume,
removing the introducer member (2114) out of the cannula (110, O1 to O3, I1 to I3) before the fluid is guided into the body (100) and/or out of the body (100).

29. Method according to claim 27 or 28, wherein the expandable arrangement is connected to the at least one lumen portion using a connection technique that connects two different parts,
preferably at least one of the following techniques: welding, soldering, using an adhesive and/or winding around or along a surface.

30. Method according to one of the claims 27 to 29, wherein the expandable arrangement comprises a plurality of wires (118), preferably comprising or consisting of a metal,
wherein proximal ends of at least two of the wires (118) or of all of the wires (118) are connected to a distal end of the lumen portion (LP), and
wherein distal ends of at least two wires, of at least three of the plurality of wires (118) or of all wires of the plurality of the wires (118) are preferably connected with each other and/or with a connection element, preferably at a position that is on the extended longitudinal axis (A) of the lumen portion (LP) of the first cannula (110, O1 to O3), and
preferably by twisting them with each other and or by arranging the distal parts of the wires parallel to the longitudinal axis of the lumen portion (LP).

31. Method according to one of the claims 27 to 30, wherein the wires (118) of the expandable arrangement of the cannula are distributed angularly such that, in a given axial position preferably in the expanded state, for a first pair of neighboring wires the wires forming the pair have a first distance relative to each other and that neighboring wires forming a second pair of neighboring wires have a second distance relative to each other that is greater than the first distance, preferably equal to or greater than twice the first distance.

32. Method according to one of the claims 27 to 31, wherein the expandable arrangement comprises a membrane, wherein the membrane is folded or less stretched in the non-expanded state of the expandable arrangement and wherein the membrane is expanded in the expanded state of the expandable arrangement.

33. Method according to one of the claims 27 to 32, wherein in its expanded state the membrane defines an opening that faces distally relative to a longitudinal axis of the lumen portion of the cannula (O1 to O3), or
wherein in its expanded state the membrane defines an opening that faces laterally relative to the longitudinal axis of the lumen portion of the first cannula (O1 to O3), or
wherein in its expanded state the membrane defines an opening that faces proximally relative to the longitudinal axis of the lumen portion of the first cannula (O1 to O3).

34. Method according to claim 32 or 33, wherein a segment of the first membrane is arranged between wires of the expandable arrangement, preferably between the wires of the second pair of wires as far as referred back to claim 31,
and wherein the segment of the first membrane has an insertion opening that is adapted to the diameter of a second cannula,
and wherein preferably the second cannula (I1 to I3) closes the main part of the insertion opening or is inserted through the insertion opening thereby closing the main part of the insertion opening.

35. Method according to one of the claims 27 to 34, wherein the cannula (110, O1 to O3, I1 to I3) is inserted endovascular to or through at least one chamber of the heart (H) and wherein the expandable arrangement is placed within the heart (H) of the subject, preferably in the left atrium (LA) or in the left ventricle (LV) of the heart (H), preferably atrial trans septal or ventricle trans septal, or within the right atrium (RA) or within the right ventricle (RV).

36. Method according to one of the claims 27 to 34, wherein the cannula (110, O1 to O3, I1 to I3) is inserted endovascular to or through at least one chamber of the heart (H) and wherein the expandable arrangement is placed within the aorta (AO) of the subject, preferably in the ascending aorta (aAO).

37. Method according to one of the claims 27 to 34, wherein the cannula (110, O1 to O3, I1 to I3) is inserted endovascular to or through at least one chamber of the heart (H) and wherein the expandable arrangement is placed within the pulmonary artery (PA) of the subject, preferably within the common pulmonary artery (PA), within the right pulmonary artery (rPA) or within the left pulmonary artery (lPA).

38. Method according to one of the claims 27 to 34, wherein the cannula (110, O1 to O3, I1 to I3) is inserted endovascular femoral and wherein the expandable arrangement is placed within the common femoral artery (CFA), within the thoracic aorta (thAO) or within the abdominal aorta (abAO), preferably transcaval.

39. Method according to one of the claims 27 to 34, wherein the cannula is inserted endovascular jugular venous, through vena cava or femoral venous and then transcaval into the descending thoracic aorta or within the abdominal aorta (abAO).

40. Method according to one of claims 27 to 39, wherein the cannula (110, O1 to O3) is a first cannula and wherein the method comprises:
providing a second cannula (I1 to I3),
wherein the second cannula (I1 to I3) is arranged within the first cannula (O1 to O3) or wherein the second cannula (I1 to I3) is inserted into the first cannula (O1 to O3) after the first cannula (O1 to O3) is within the body (100) or
wherein the first cannula is arranged within the second cannula or wherein the second cannula is inserted into the body (100) before the first cannula is inserted through the second cannula into the body (100).

41. Method according to claim 40, wherein the second cannula (I1 to I3) comprises a flexible lumen portion that extends axially between a proximal part of the second cannula (I1 to I3) and a distal part of the second cannula (I1 to I3), and/or
wherein the second cannula (I1 to I3) carries a second expandable arrangement, preferably an expandable arrangement that comprises wires.

42. Method according to claim 40 or 41 wherein the second cannula (I1 to I3) is inserted using an introducer member (2114).

43. Method according to claim 41 or 42, wherein the second expandable arrangement comprises a membrane, wherein the membrane of the second expandable arrangement is folded or less stretched in the non-expanded state of the expandable arrangement of the second expandable arrangement and wherein the membrane of the second expandable arrangement is expanded in the expanded state of the second expandable arrangement.

44. Method according to claim 43, wherein in its expanded state the membrane of the second expandable arrangement defines an opening that faces distally relative to a longitudinal axis of the second cannula (I1 to I3) or that faces transversally relative to the longitudinal axis of the second cannula (I1 to I3) or that faces proximally relative to the longitudinal axis of the second cannula (I1 to I3).

45. Method according to one of the claims 40 to 44, wherein the first cannula (O1 to O3) is inserted endovascular through the right atrium (RA), the atrial septum into the left atrium (LA) where the first cannula (O1 to O3) is fixed by the expandable arrangement of the first cannula (O1 to O3),
and wherein the second cannula (I1 to I3) is inserted through the first cannula (O1 to O3) and the expandable arrangement of the first cannula into the left ventricle (LV) and then into the aorta (AO), preferably into the ascending aorta (aAO), where the second cannula (I1 to I3) is fixed by the second expandable arrangement.

46. Method according to one of the claims 40 to 44, wherein the first cannula (O1 to O3) is inserted endovascular through the superior vena cava (SVC) into the right atrium (RA) of the heart (H) or into the right ventricle (RV) where preferably the first cannula (O1 to O3) is fixed by the expandable arrangement of the first cannula (O1 to O3),
and wherein the second cannula (I1 to I3) is inserted through the first cannula (O1 to O3) and preferably through the optional expandable arrangement of the first cannula (O1 to O3) into the common pulmonary artery (PA), into the left pulmonary artery (lPA) or into the right pulmonary artery (rPA) where the second cannula (I1 to I3) is fixed by the expandable arrangement of the second cannula (I1 to I3).

47. Method according to claim 46, wherein the first cannula comprises a first group of holes that is arranged in the right ventricle and/or wherein the first cannula comprises a second group of holes that is arranged within the right atrium,
wherein preferably the first group of holes and/or the second group of holes are used for drainage of fluid or blood from the heart,
wherein preferably a portion of at least 1 cm, at least 1.5 cm or of at least 2 cm axial length and without holes is arranged between the first group of holes and the second group of holes.

48. Method according to claim 46 or 47, wherein a third cannula (O1 to O3) is inserted endovascular through the right atrium (RA), the atrial septum into the left atrium (LA) where the third cannula is fixed by an expandable arrangement of the third cannula (O1 to O3),
and wherein a fourth cannula (I1 to I3) is inserted through the third cannula (O1 to O3) and the expandable arrangement of the third cannula into the left ventricle (LV) and then into the aorta (AO), preferably into the ascending aorta (aAO), where the fourth cannula is fixed by an expandable arrangement of the fourth cannula.

49. Method according to any one of the claims 40 to 48, wherein at least one sealing element (S1, S2) is arranged within an opening (OP1, OP2) through which the second cannula (I1 to I3) is inserted into the first cannula (O1 to O3), preferably a retaining ring, a sealing ring, a gasket or a multi-flap valve or another self-sealing element.

50. Method according to any one of the claims 40 to 49, wherein a closure element is arranged at the distal part of the first cannula (O1 to O3) that prevents the passage of fluid through the distal end of the first cannula (O1 to O3) into the first lumen of the first cannula (O1 to O3) and/or vice versa,
preferably a closure element that allows the passage of the second cannula (I1 to I3) and/or of an introducer member (2114), preferably a multi-flap valve or another self-sealing element.

51. Method according to one of the claims 27 to 50, wherein the first fluid and/or the second fluid is injected into the body (100) and/or taken out of the body (100) in a pulsed fluid flow or in a continuous fluid flow.

52. Method according to one of the claims 27 to 51, wherein the first cannula (O1 to O3) and/or the second cannula (I1 to I3) is pre-bended or both cannulas (O1 to O3, I1 to I3) are pre-bended by an angle (W1) within the range of 60 degrees to 175 degrees or of 70 degrees to 145 degrees, preferably in order to ease an insertion through the septum of the heart (H) of the subject, preferably through the atrial septum or through the ventricle septum.

53. Method according to one of the claims 27 to 52, wherein a cannula (110, O1 to O3, I1 to I2) according to one of the claims 1 to 20 is used and/or wherein a cannula system (CS1 to CS3) according to one of the claims 21 to 26 is used.
